SEQUENCE LISTING (106 sequences)

SEQ ID NO: 1 - DNA, 20 bp, Artificial Sequence
  A synthetic oligonucleotide
tgtaatcccagcactttggg

SEQ ID NO: 2 - DNA, 20 bp, Artificial Sequence
  A synthetic oligonucleotide
tcccaaagtgctgggattac

SEQ ID NO: 3 - DNA, 20 bp, Artificial Sequence
  A synthetic oligonucleotide
gcctgtaatcccagcacttt

SEQ ID NO: 4 - DNA, 20 bp, Artificial Sequence
  A synthetic oligonucleotide
cgcctgtaatcccagcactt

SEQ ID NO: 5 - DNA, 20 bp, Artificial Sequence
  A synthetic oligonucleotide
cctcggcctcccaaagtgct

SEQ ID NO: 6 - DNA, 20 bp, Artificial Sequence
  A synthetic oligonucleotide
cccagcactttgggaggccg

SEQ ID NO: 7 - DNA, 20 bp, Artificial Sequence
  A synthetic oligonucleotide
ctcccaaagtgctgggatta

SEQ ID NO: 8 - DNA, 20 bp, Artificial Sequence
  A synthetic oligonucleotide
ctgtaatcccagcactttgg

SEQ ID NO: 9 - DNA, 20 bp, Artificial Sequence
  A synthetic oligonucleotide
tcccagcactttgggaggcc

SEQ ID NO: 10 - DNA, 20 bp, Artificial Sequence
  A synthetic oligonucleotide
ttctcctgcctcagcctccc

SEQ ID NO: 11 - DNA, 20 bp, Artificial Sequence
  A synthetic oligonucleotide
agtgagttccaggacagcca

SEQ ID NO: 12 - DNA, 20 bp, Artificial Sequence
  A synthetic oligonucleotide
ttgttccacctatagggttg

SEQ ID NO: 13 - DNA, 20 bp, Artificial Sequence
  A synthetic oligonucleotide
ctttctctagctcctccatt

SEQ ID NO: 14 - DNA, 20 bp, Artificial Sequence
  A synthetic oligonucleotide
cccaatggaggagctagaga

SEQ ID NO: 15 - DNA, 20 bp, Artificial Sequence
  A synthetic oligonucleotide
ccattctgactggtgtgaga

SEQ ID NO: 16 - DNA, 20 bp, Artificial Sequence
  A synthetic oligonucleotide
gaagtcctagccagagcaat

SEQ ID NO: 17 - DNA, 20 bp, Artificial Sequence
  A synthetic oligonucleotide
attgctctggctaggacttc

SEQ ID NO: 18 - DNA, 20 bp, Artificial Sequence
  A synthetic oligonucleotide
gtctcccactattattgtgt

SEQ ID NO: 19 - DNA, 20 bp, Artificial Sequence
  A synthetic oligonucleotide
ttgaatctgtagattgcttt

SEQ ID NO: 20 - DNA, 20 bp, Artificial Sequence
  A synthetic oligonucleotide
cctcccaagtgctgggatta

SEQ ID NO: 21 - DNA, 20 bp, Artificial Sequence
  A synthetic oligonucleotide
aagaaagaaagaaagaaaga

SEQ ID NO: 22 - DNA, 20 bp, Artificial Sequence
  A synthetic oligonucleotide
gagagagagagagagagaga

SEQ ID NO: 23 - DNA, 20 bp, Artificial Sequence
  A synthetic oligonucleotide
aggaaggaaggaaggaagga

SEQ ID NO: 24 - DNA, 20 bp, Artificial Sequence
  A synthetic oligonucleotide
tagatagatagatagataga

SEQ ID NO: 25 - DNA, 20 bp, Artificial Sequence
  A synthetic oligonucleotide
cacacacacacacacacaca

SEQ ID NO: 26 - DNA, 20 bp, Artificial Sequence
  A synthetic oligonucleotide
tggatggatggatggatgga

SEQ ID NO: 27 - DNA, 20 bp, Artificial Sequence
  A synthetic oligonucleotide
agtaatcccagcactttggg

SEQ ID NO: 28 - DNA, 20 bp, Artificial Sequence
  A synthetic oligonucleotide
gggctggagagatggctcag

SEQ ID NO: 29 - DNA, 20 bp, Artificial Sequence
  A synthetic oligonucleotide
ggccaaacgtgccctgacgg

SEQ ID NO: 30 - DNA, 20 bp, Artificial Sequence
  A synthetic oligonucleotide
gcgatgggggggtgggtagc

SEQ ID NO: 31 - DNA, 20 bp, Artificial Sequence
  A synthetic oligonucleotide
gacgactagttaggcgtgta

SEQ ID NO: 32 - DNA, 20 bp, Artificial Sequence
  A synthetic oligonucleotide
cattattctagtgtcacgcc

SEQ ID NO: 33 - DNA, 20 bp, Artificial Sequence
  A synthetic oligonucleotide
gggtatgaagtttggtgtcc

SEQ ID NO: 34 - DNA, 20 bp, Artificial Sequence
  A synthetic oligonucleotide
aatggtcagattgccaaaga

SEQ ID NO: 35 - DNA, 20 bp, Artificial Sequence
  A synthetic oligonucleotide
acaaaagtcagatccgtacc

SEQ ID NO: 36 - DNA, 20 bp, Artificial Sequence
  A synthetic oligonucleotide
tacctggagcaactcccgag

SEQ ID NO: 37 - DNA, 20 bp, Artificial Sequence
  A synthetic oligonucleotide
actttcgtcaaccagttcta

SEQ ID NO: 38 - protein, 1368 aa, Streptococcus pyogenes [sequence omitted]

SEQ ID NO: 39 - DNA, 4101 bp, Streptococcus pyogenes
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgac

SEQ ID NO: 40 - protein, 1307 aa, Francisella novicida
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 41 - DNA, 3921 bp, Francisella novicida
atgacacagttcgagggctttaccaacctgtatcaggtgagcaagacactgcggtttgagctgatcccacagggcaagaccctgaagcacatccaggagcagggcttcatcgaggaggacaaggcccgcaatgatcactacaaggagctgaagcccatcatcgatcggatctacaagacctatgccgaccagtgcctgcagctggtgcagctggattgggagaacctgagcgccgccatcgactcctatagaaaggagaaaaccgaggagacaaggaacgccctgatcgaggagcaggccacatatcgcaatgccatccacgactacttcatcggccggacagacaacctgaccgatgccatcaataagagacacgccgagatctacaagggcctgttcaaggccgagctgtttaatggcaaggtgctgaagcagctgggcaccgtgaccacaaccgagcacgagaacgccctgctgcggagcttcgacaagtttacaacctacttctccggcttttatgagaacaggaagaacgtgttcagcgccgaggatatcagcacagccatcccacaccgcatcgtgcaggacaacttccccaagtttaaggagaattgtcacatcttcacacgcctgatcaccgccgtgcccagcctgcgggagcactttgagaacgtgaagaaggccatcggcatcttcgtgagcacctccatcgaggaggtgttttccttccctttttataaccagctgctgacacagacccagatcgacctgtataaccagctgctgggaggaatctctcgggaggcaggcaccgagaagatcaagggcctgaacgaggtgctgaatctggccatccagaagaatgatgagacagcccacatcatcgcctccctgccacacagattcatccccctgtttaagcagatcctgtccgataggaacaccctgtctttcatcctggaggagtttaagagcgacgaggaagtgatccagtccttctgcaagtacaagacactgctgagaaacgagaacgtgctggagacagccgaggccctgtttaacgagctgaacagcatcgacctgacacacatcttcatcagccacaagaagctggagacaatcagcagcgccctgtgcgaccactgggatacactgaggaatgccctgtatgagcggagaatctccgagctgacaggcaagatcaccaagtctgccaaggagaaggtgcagcgcagcctgaagcacgaggatatcaacctgcaggagatcatctctgccgcaggcaaggagctgagcgaggccttcaagcagaaaaccagcgagatcctgtcccacgcacacgccgccctggatcagccactgcctacaaccctgaagaagcaggaggagaaggagatcctgaagtctcagctggacagcctgctgggcctgtaccacctgctggactggtttgccgtggatgagtccaacgaggtggaccccgagttctctgcccggctgaccggcatcaagctggagatggagccttctctgagcttctacaacaaggccagaaattatgccaccaagaagccctactccgtggagaagttcaagctgaactttcagatgcctacactggcctctggctgggacgtgaataaggagaagaacaatggcgccatcctgtttgtgaagaacggcctgtactatctgggcatcatgccaaagcagaagggcaggtataaggccctgagcttcgagcccacagagaaaaccagcgagggctttgataagatgtactatgactacttccctgatgccgccaagatgatcccaaagtgcagcacccagctgaaggccgtgacagcccactttcagacccacacaacccccatcctgctgtccaacaatttcatcgagcctctggagatcacaaaggagatctacgacctgaacaatcctgagaaggagccaaagaagtttcagacagcctacgccaagaaaaccggcgaccagaagggctacagagaggccctgtgcaagtggatcgacttcacaagggattttctgtccaagtataccaagacaacctctatcgatctgtctagcctgcggccatcctctcagtataaggacctgggcgagtactatgccgagctgaatcccctgctgtaccacatcagcttccagagaatcgccgagaaggagatcatggatgccgtggagacaggcaagctgtacctgttccagatctataacaaggactttgccaagggccaccacggcaagcctaatctgcacacactgtattggaccggcctgttttctccagagaacctggccaagacaagcatcaagctgaatggccaggccgagctgttctaccgccctaagtccaggatgaagaggatggcacaccggctgggagagaagatgctgaacaagaagctgaaggatcagaaaaccccaatccccgacaccctgtaccaggagctgtacgactatgtgaatcacagactgtcccacgacctgtctgatgaggccagggccctgctgcccaacgtgatcaccaaggaggtgtctcacgagatcatcaaggataggcgctttaccagcgacaagttctttttccacgtgcctatcacactgaactatcaggccgccaattccccatctaagttcaaccagagggtgaatgcctacctgaaggagcaccccgagacacctatcatcggcatcgatcggggcgagagaaacctgatctatatcacagtgatcgcctccaccggcaagatcctggagcagcggagcctgaacaccatccagcagtttgattaccagaagaagctggacaacagggagaaggagagggtggcagcaaggcaggcctggtctgtggtgggcacaatcaaggatctgaagcagggctatctgagccaggtcatccacgagatcgtggacctgatgatccactaccaggccgtggtggtgctggagaacctgaatttcggctttaagagcaagaggaccggcatcgccgcgaaggccgtgtaccagcagttcgagaagatgctgatcgataagctgaattgcctggtgctgaaggactatccagcagagaaagtgggaggcgtgctgaacccataccagctgacagaccagttcacctcctttgccaagatgggcacccagtctggcttcctgttttacgtgcctgccccatatacatctaagatcgatcccctgaccggcttcgtggaccccttcgtgtggaaaaccatcaagaatcacgagagccgcaagcacttcctggagggcttcgactttctgcactacgacgtgaaaaccggcgacttcatcctgcactttaagatgaacagaaatctgtccttccagaggggcctgcccggctttatgcctgcatgggatatcgtgttcgagaagaacgagacacagtttgacgccaagggcacccctttcatcgccggcaagagaatcgtgccagtgatcgagaatcacagattcaccggcagataccgggacctgtatcctgccaacgagctgatcgccctgctggaggagaagggcatcgtgttcagggatggctccaacatcctgccaaagctgctggagaatgacgattctcacgccatcgacaccatggtggccctgatccgcagcgtgctgcagatgcggaactccaatgccgccacaggcgaggactatatcaacagccccgtgcgcgatctgaatggcgtgtgcttcgactcccggtttcagaacccagagtggcccatggacgccgatgccaatggcgcctaccacatcgccctgaagggccagctgctgctgaatcacctgaaggagagcaaggatctgaagctgcagaacggcatctccaatcaggactggctggcctacatccaggagctgcgcaac

SEQ ID NO: 42 - protein, 1368 aa, Streptococcus pyogenes [sequence omitted]

SEQ ID NO: 43 - protein, 1085 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 44 - protein, 1410 aa, Artificial Sequence
  A synthetic amino acid sequence
MAPKKKRKVSANPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPTSPKKKRKV

SEQ ID NO: 45 - protein, 1410 aa, Artificial Sequence
  A synthetic amino acid sequence
MAPKKKRKVSATGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGTSPKKKRKV

SEQ ID NO: 46 - protein, 1410 aa, Artificial Sequence
  A synthetic amino acid sequence
MAPKKKRKVSANGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGTSPKKKRKV

SEQ ID NO: 47 - protein, 1410 aa, Artificial Sequence
  A synthetic amino acid sequence
MAPKKKRKVSAKIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEITSPKKKRKV

SEQ ID NO: 48 - protein, 1410 aa, Artificial Sequence
  A synthetic amino acid sequence
MAPKKKRKVSATEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPETSPKKKRKV

SEQ ID NO: 49 - protein, 1410 aa, Artificial Sequence
  A synthetic amino acid sequence
MAPKKKRKVSAIADANLDKVLSAYNKHRDKPIREQAENIIHLFTLTNLGAPAAFKYFDTTIDRKRYTSTKEVLDATLIHQSITGLYETRIDLSQLGGDGGSGGSGGSGGSGGSGGSGGMDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILATSPKKKRKV

SEQ ID NO: 50 - protein, 1395 aa, Flavivirus virus
MAPKKKRKVSANPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKQKKRGGKDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEETSPKKKRKV

SEQ ID NO: 51 - protein, 8 aa, Artificial Sequence
  A synthetic amino acid sequence
DYKDDDDK

SEQ ID NO: 52 - DNA, 20 bp, Artificial Sequence
  A synthetic oligonucleotide sequence
cctcgaacttcacctcggcg

SEQ ID NO: 53 - DNA, 20 bp, Artificial Sequence
  A synthetic oligonucleotide sequence
caactacaagacccgcgccg

SEQ ID NO: 54 - DNA, 20 bp, Artificial Sequence
  A synthetic oligonucleotide sequence
ccggcaagctgcccgtgccc

SEQ ID NO: 55 - DNA, 20 bp, Artificial Sequence
  A synthetic oligonucleotide sequence
cattattctagtgtcacgcc

SEQ ID NO: 56 - DNA, 20 bp, Artificial Sequence
  A synthetic oligonucleotide sequence
gggtatgaagtttggtgtcc

SEQ ID NO: 57 - DNA, 20 bp, Artificial Sequence
  A synthetic oligonucleotide sequence
ccggtggtcactctgtatgc

SEQ ID NO: 58 - DNA, 20 bp, Artificial Sequence
  A synthetic oligonucleotide sequence
tgtcgagcccgacgcgcgtg

SEQ ID NO: 59 - DNA, 20 bp, Artificial Sequence
  A synthetic oligonucleotide sequence
gctcggtgacccgctcgatg

SEQ ID NO: 60 - DNA, 20 bp, Artificial Sequence
  A synthetic oligonucleotide sequence
acaaaagtcagatccgtacc

SEQ ID NO: 61 - skipped

SEQ ID NO: 62 - DNA, 20 bp, Artificial Sequence
  A synthetic oligonucleotide sequence
tacctggagcaactcccgag

SEQ ID NO: 63 - DNA, 20 bp, Artificial Sequence
  A synthetic oligonucleotide sequence
cgcctgtaatcccagcactt

SEQ ID NO: 64 - DNA, 20 bp, Artificial Sequence
  A synthetic oligonucleotide sequence
cctcggcctcccaaagtgct

SEQ ID NO: 65 - DNA, 20 bp, Artificial Sequence
  A synthetic oligonucleotide sequence
tgtaatcccagcactttggg

SEQ ID NO: 66 - DNA, 20 bp, Artificial Sequence
  A synthetic oligonucleotide sequence
tcccaaagtgctgggattac

SEQ ID NO: 67 - protein, 10 aa, Artificial Sequence
  A synthetic amino acid sequence
PDNSDVDKLF

SEQ ID NO: 68 - protein, 10 aa, Artificial Sequence
  A synthetic amino acid sequence
QLFEENPINA

SEQ ID NO: 69 - protein, 10 aa, Artificial Sequence
  A synthetic amino acid sequence
LIAQLPGEKK

SEQ ID NO: 70 - protein, 10 aa, Artificial Sequence
  A synthetic amino acid sequence
QLSKDTYDDD

SEQ ID NO: 71 - protein, 10 aa, Artificial Sequence
  A synthetic amino acid sequence
ILRVNTEITK

SEQ ID NO: 72 - protein, 10 aa, Artificial Sequence
  A synthetic amino acid sequence
ESEFVYGDYK

SEQ ID NO: 73 - protein, 10 aa, Artificial Sequence
  A synthetic amino acid sequence
GDYKVYDVRK

SEQ ID NO: 74 - protein, 10 aa, Artificial Sequence
  A synthetic amino acid sequence
VRKMIAKSEQ

SEQ ID NO: 75 - protein, 10 aa, Artificial Sequence
  A synthetic amino acid sequence
KSEQEIGKAT

SEQ ID NO: 76 - protein, 10 aa, Artificial Sequence
  A synthetic amino acid sequence
YFFYSNIMNF

SEQ ID NO: 77 - protein, 10 aa, Artificial Sequence
  A synthetic amino acid sequence
YEKLKGSPED

SEQ ID NO: 78 - protein, 10 aa, Artificial Sequence
  A synthetic amino acid sequence
KLKGSPEDNE

SEQ ID NO: 79 - protein, 10 aa, Artificial Sequence
  A synthetic amino acid sequence
SKRVILADAN

SEQ ID NO: 80 - protein, 8 aa, Artificial Sequence
  A synthetic amino acid sequence
KQKKRGGK

SEQ ID NO: 81 - protein, 7 aa, Artificial Sequence
  A synthetic amino acid sequence
PKKKRKV

SEQ ID NO: 82 - protein, 16 aa, Artificial Sequence
  A synthetic amino acid sequence
KRPAATKKAGQAKKKK

SEQ ID NO: 83 - protein, 20 aa, Artificial Sequence
  A synthetic amino acid sequence
AVKRPAATKKAGQAKKKKLD

SEQ ID NO: 84 - protein, 25 aa, Artificial Sequence
  A synthetic amino acid sequence
MSRRRKANPTKLSENAKKLAKEVEN

SEQ ID NO: 85 - protein, 9 aa, Artificial Sequence
  A synthetic amino acid sequence
PAAKRVKLD

SEQ ID NO: 86 - protein, 9 aa, Artificial Sequence
  A synthetic amino acid sequence
KLKIKRPVK

SEQ ID NO: 87 - protein, 8 aa, Artificial Sequence
  A synthetic amino acid sequence
LEVLFQGP

SEQ ID NO: 88 - protein, 8 aa, Artificial Sequence
  A synthetic amino acid sequence
KERKRRGA

SEQ ID NO: 89 - protein, 8 aa, Artificial Sequence
  A synthetic amino acid sequence
SSRKRRSH

SEQ ID NO: 90 - protein, 8 aa, Artificial Sequence
  A synthetic amino acid sequence
NRRRRSAG

SEQ ID NO: 91 - protein, 7 aa, Artificial Sequence
  A synthetic amino acid sequence
LKRRSGS

SEQ ID NO: 92 - protein, 8 aa, Artificial Sequence
  A synthetic amino acid sequence
QVVVHQSK

SEQ ID NO: 93 - protein, 7 aa, Artificial Sequence
  A synthetic amino acid sequence
ENLYFQS

SEQ ID NO: 94 - protein, 7 aa, Artificial Sequence
  A synthetic amino acid sequence
GGCSHQS

SEQ ID NO: 95 - protein, 8 aa, Artificial Sequence
  A synthetic amino acid sequence
YDVRHQSR

SEQ ID NO: 96 - protein, 8 aa, Artificial Sequence
  A synthetic amino acid sequence
GLEVEQGR

SEQ ID NO: 97 - protein, 8 aa, Artificial Sequence
  A synthetic amino acid sequence
EGVGHQSK

SEQ ID NO: 98 - protein, 10 aa, Artificial Sequence
  A synthetic amino acid sequence
VGHQSVGHQS

SEQ ID NO: 99 - protein, 8 aa, Artificial Sequence
  A synthetic amino acid sequence
KQKKRGGK

SEQ ID NO: 100 - protein, 7 aa, Artificial Sequence
  A synthetic amino acid sequence
LKRRSGS

SEQ ID NO: 101 - protein, 4 aa, Artificial Sequence
  A synthetic amino acid sequence
NGCG

SEQ ID NO: 102 - protein, 4 aa, Artificial Sequence
  A synthetic amino acid sequence
NGAG

SEQ ID NO: 103 - protein, 4 aa, Artificial Sequence
  A synthetic amino acid sequence
NGAN

SEQ ID NO: 104 - protein, 4 aa, Artificial Sequence
  A synthetic amino acid sequence
NGNG

SEQ ID NO: 105 - protein, 6 aa, Artificial Sequence
  A synthetic amino acid sequence
NNGRRT

SEQ ID NO: 106 - protein, 6 aa, Artificial Sequence
  A synthetic amino acid sequence
NNNRRT

(19) United States
(12) Patent Application Publication
(10) Pub. No.: US 2023/0043813 A1
(43) Pub. Date: Feb. 9, 2023

(54) METHODS FOR TARGETED CELL DEPLETION

(71) Applicants: The Regents of the University of California, Oakland, CA (US); The J. David Gladstone Institutes, a testamentary trust established under the Will of J. David, San Francisco, CA (US)

(72) Inventor: Christof Fellmann, San Francisco, CA (US)

(21) Appl. No.: 17/765,583

(22) PCT Filed: Oct. 2, 2020
(86) PCT No.: PCT/US2020/053896

§ 371 Date: Mar. 31, 2022

Related U.S. Application Data
(60) Provisional application No. 62/910,558, filed on Oct. 4, 2019.

(51) Int. Cl. C12N 15/11; C12N 9/22; A61K 48/00

(52) U.S. Cl. CPC C12N 15/11; C12N 9/22; A61K 48/005; C12N 2310/20; C12N 2740/15043; C12N 2800/80; C07K 2319/50

(57) ABSTRACT

Described herein are compositions, kits and methods for shredding the genomes of selected cell types, for example, the genomes of selected cancer cell types.

### CROSS-REFERENCE

This application claims benefit of priority to the filing of U.S. Provisional Application Ser. No. 62/910,558, filed Oct. 4, 2019, the contents of which are specifically incorporated herein by reference in their entirety.

### GOVERNMENT FUNDING

This invention was made with government support under R00GM118909 awarded by the National Institutes of Health. The government has certain rights in the invention.

### INCORPORATION BY REFERENCE OF SEQUENCE LISTING PROVIDED AS A TEXT FILE

A Sequence Listing is provided herewith as a text file, “3730037WO1SEQ LIST.txt” created on Sep. 30, 2020 and having a size of 143,894 bytes. The contents of the text file are incorporated by reference herein in their entirety.

### BACKGROUND

Although strides have been made in the treatment of cancer, treatment options for many types of cancer are not optimal. For example, glioblastoma (GBM) is the most common and lethal primary brain tumor in adults. Despite aggressive treatment regimens including surgical resection, radiotherapy, and chemotherapy, the median survival remains only 12-15 months. Glioblastomas are highly diffuse and infiltrate the normal brain, rendering complete resection complicated or impossible. The growth of residual tumor often results in therapy resistance and ultimately death. Additionally, recent genomic studies have revealed that glioblastomas exhibit extensive intratumoral heterogeneity, with various subpopulations of cells harboring distinct mutations and displaying diverse epigenetic states. Similar issues exist for other types of cancer.

Therefore, a need exists to establish innovative treatment strategies that can target and efficiently eliminate cancer cells in vivo irrespective of their mutational and epigenetic profile.

### SUMMARY

Described herein are methods and compositions for depleting or eliminating cells that involve CRISPR-Cas mediated targeting and cutting of repetitive or highly repetitive sequences in the genomes of cancer cells, also referred to herein as “Genome Shredding.” The methods and compositions result in the fragmentation of a target cell's genome and DNA damage-induced cell death, hence providing a genotype/mutation-agnostic treatment paradigm. For example, by introducing Cas enzymes into cancer cells, an adaptive immune response is stimulated that create a pro-inflammatory/anti-tumor immune microenvironment that further assists tumor clearance and remission. The methods can be performed in vitro and in vivo.

### DESCRIPTION OF THE FIGURES

FIG. 1A-1I illustrates an unbiased Cas9 library screen that identifies active circularly permuted Cas9 (Cas9-CP) proteins. FIG. 1A schematically illustrates circular permutation and library generation for Cas9. FIG. 1B graphically illustrates enrichment values of functional Cas9-CP library members generated by the unbiased screen as determined by flow cytometry and colony-forming units (CFU) that express green fluorescent protein (GFP). Error bars represent standard deviation in all panels. FIG. 1C graphically illustrates deep-sequencing read averages for pre-Cas9-circular permutant and post-Cas9-circular permutant library members, demonstrating a strong clustering of highly enriched library members with internal (within 4 amino acid of the N and C termini) and empirically validated controls. The dotted line highlights an approximate boundary that represents >100-fold enrichment in the screen. FIG. 1D is a schematic diagram of the Cas sequence showing locations of Cas9-CP termini (vertical lines) with the Cas9 domains identified. FIG. 1E graphically illustrates the activities of deactivated Cas9 circular permutant (dCas9-CP) proteins with different endpoint values as detected using a 12-hr E. coli CRISPRi DNA binding and red fluorescence protein (RFP) repression system. Wild type dCas9 and a protein expression vector control are also shown. The values are for triplicate assays (error bars represent SD; *p<0.05; ns, not significant, t test). FIG. 1F graphically illustrates the activities of deactivated Cas9 circular permutant (dCas9-CP) proteins as reported by CFU/mL readings in an E. coli genomic cleavage assay readout of cell death compared with a protein expression vector control, WT dCas9, and WT Cas9 (n=3, error bars represent SD; *p<0.05; ns, not significant, t test). FIG. 1G graphically illustrates the activities of deactivated Cas9 circular permutant (dCas9-CP) proteins as reported by cleavage efficiency of a genomic reporter in mammalian cells in triplicate (illustrated in FIG. 1H), observed via indel formation, and GFP reporter disruption. hCas9 is human codon-optimized Cas9; bCas9 indicates bacterial codon-based Cas9 constructs (error bars represent SD; *p<0.05; ns, not significant, t test). FIG. 1H schematically illustrates a rapid mammalian genome editing reporter assay. Monoclonal reporter cell lines were established by stably integrating an all-in-one Tet-On cassette enabling doxycycline-inducible GFP expression, followed by selection and characterization of single clones. To assess editing efficiency of novel variants, reporter cells are transduced with Cas constructs of interest and guide RNAs targeting GFP, or a non-targeting control. At 24+ hours post-transduction, the GFP fluorescence reporter is induced by doxycycline treatment for 24-48 hours and genome shredding was quantified by flow cytometry. FIG. 1I is a schematic illustrating the transposon method of building Cas-CP libraries. The REs abbreviation refers to Restriction Enzyme sites.

FIG. 2A-2D illustrates that linker length can be utilized to control Cas9-CP activity. FIG. 2A illustrates the effect of linker length on Cas9-CP activity in an endpoint analysis of an E. coli CRISPRi-based GFP repression assay run in triplicate using Cas9-CPs identified as functional with 20 amino acid linkers, then evaluated with GGSₙ linkers of length 5, 10, 15, 20, 25, and 30 amino acids. Error bars represent standard deviation in all panels. FIG. 2B is a schematic illustrating the rationale behind using a Cas9-CP with a short amino acid linker to provide a “caged” Cas9-CP molecule. FIG. 2C graphically illustrates Cas9-CP activities in a CP-endpoint analysis involving an E. coli CRISPRi-based GFP expression time course for six Cas9-CPs containing a 7-amino acid tobacco etch virus (TEV) linker (ENLYFQ/S) in the presence of a functional TEV protease (TEV, hatched bars) compared with deactivated TEV protease with the catalytic triad mutant C151A (dTEV, clear bars). Data for a defective Cas-9-CP without the TEV linker is shown for comparison. The assays were performed in triplicate (n=3, error bars represent SD; *p<0.05; ns, not significant, t test). FIGS. 2D-1 and 2D-2 illustrate by western blot analysis that the sizes of different circularly permuted Cas proteins (Cas9-CPs) correlate with their determined sequences. FIG. 2D-1 is a schematic diagram of the Cas9-CP structures. FIG. 2D-2 shows western blots of the Cas9-CPs using the Flag epitope on the C terminus of the CP-TEVs after the endpoint measurement as shown in FIG. 2C. Expected kilodaltons shown to the right indicate the predicted band size if cleavages occur at the TEV site in the CP linker region.

FIG. 3A-3L illustrate which ProCas9s optimally respond to cleavage via (e.g., sensing and responding to) Polyvirus and Flavivirus Proteases. FIG. 3A graphically illustrates that Cas9-CP199 had the greatest Cas9 response (difference in specific versus non-specific protease cleavage) as measured by endpoint analysis in an E. coli CRISPRi based GFP expression assay of the six Cas9-CPs designed to contain an eight-amino acid 3C linker (LEVLFQ/GP (SEQ ID NO:87) in the presence of a functional 3C protease (3C pro, hatched bars) or a deactivated TEV protease with a catalytic triad mutant C151A (dProtease, clear bars). FIG. 3B shows a heatmap depicting the fold activation of a suite of ProCas9 CP linkers (shown in Table 4) for Potyviral N1a proteases. Data are normalized to a non-active protein expression control (dTEV) in an E. coli-based. CRISPRi GFP repression assay. Darker coloration indicates greater activity (n=2). FIG. 3C graphically illustrates analysis of different NIa proteases for release of Cas9 activities by cleavage of the QVVVHQSK linker derived from Plum Pox virus (PPV) using the E. coli CRISPRi assay. Cleavage by a dead protease (dProtease) is shown for comparison. Assays were performed in triplicate (n=3, error bars represent SD; *p<0.05; ns, not significant, t test compared to dProtease). FIG. 3D shows a heatmap depicting Cas9 activation by different Flavivirus NS2B-NS3 proteases when different ProCas9 CP linkers (shown in Table 4) are used. An E. coli-based CRISPRi GFP repression assay was used and the data are normalized to a non-active protein expression control (deactivated TEV, dTEV protease). Darker coloration indicates greater activity (n 2). FIG. 3E graphically illustrates Cas9 activation initiated by cleavage of a linker derived from West Nile virus (WNV, see Table 4) by different NS2B-NS3 proteases. These results were from an endpoint analysis using the E. coli CRISPRi assay; the response of the distinct NS2B-NS3 proteases was compared to that of a dead protease (dProtease) (n=3, error bars represent SD; *p<0.05; ns, not significant; t test compared to dProtease). FIG. 3F shows a schematic diagram illustrating the constructs used for the transient transfection and testing in HEK293T cells of different protease/Cas9CP-linker combinations. FIG. 3G illustrates Cas9 activities when different guide RNAs (specific and not specific for target) are used in mammalian GFP disruption assays of ProCas9 enzymes with polyvirus cleavage sites in HEK293T-based reporter cells. The cells were transfected with vectors expressing the indicated sgRNAs, with an indicated WT Cas9 protein or ProCas9 protein variant, and with the indicated protease. The proteases tested included the deactivated protease (dProtease), turnip mosaic virus (TuMV) protease, plum pox virus (PPV) protease, potato virus Y (PVY) protease. Zika virus (ZIKV) protease, West Nile virus (WNV, Kunjin strain) protease). Reduction in GFP-positive cells indicates genome cleavage by a Cas9 construct (n=3; error bars represent SD; *p<0.05, t test compared to dProtease). FIG. 3H illustrates Cas9 activities when different guide RNAs (specific and not specific for target) are used in mammalian GFP disruption assays of ProCas9 enzymes with flavivirus cleavage sites in HEK293T-based reporter cells. The cells were transfected with vectors expressing the indicated sgRNAs, with WT Cas9 protein or a ProCas9 protein variant, and with the indicated protease (deactivated protease (dProtease), turnip mosaic virus (TuMV) protease, plum pox virus (PPV) protease, potato virus Y (PVY) protease, Zika virus (ZIKV) protease, West Nile virus (WNV, Kunjin strain) protease). Reduction in GFP-positive cells indicated genome cleavage by a Cas9 construct n=3; error bars represent SD; *p<0.05, t test compared to dProtease). FIG. 3I graphically illustrates leakiness and orthogonality of the original and shortened ProCas9Flavi constructs. The percentage of GFP disruption with normalization to the nontargeting guide is shown for each construct-protease pairing. In addition to the deactivated protease (dProtease) control, the active Potyvirus Ma proteases were used to assess orthogonality (n=3; error bars represent SD; *p<0.05; ns, not significant, t test). FIG. 3J shows flow cytometry plots from FIG. 3F with overlay of GFP-targeting (solid line) versus non-targeting (dashed lines) ProCas9Flavi systems, demonstrating a small degree of background activity. FIG. 3K is a schematic diagram illustrating the structure of a circularly permuted Cas protein with a truncation of the ProCas9 amino acid linker to prevent leakiness. FIG. 3L graphically illustrates GFP disruption as a measure of leakiness and orthogonality of the original and shortened ProCas9Flavi constructs. Data are displayed as a percentage of GFP signal disrupted with normalization to the nontargeting guide for each construct-protease pairing. In addition to the deactivated protease (dProtease) control, the active Potyvirus NIa proteases were used to assess orthogonality (n=3; error bars represent SD; *p<0.05; ns, not significant, t test). (SEQ ID NOs: 88-99)

FIG. 4A-4K illustrates that ProCas9 stably integrated into mammalian genomes can sense and respond to flavivirus proteases. FIG. 4A schematically illustrates genomic integration and testing of Flavivirus protease-sensitive ProCas9s. HEK-RT1 genome editing reporter cells were stably transduced with various ProCas9 lentiviral vectors, followed by puromycin selection of ProCas9 cell lines. These cell lines are then (1) tested for leaky ProCas9 activity in the absence of a stimulus or (2) stably transduced with a vector expressing the indicated proteases, followed by assessment of genome editing using the GFP reporter. FIG. 4B graphically illustrates leakiness of ProCas9 variants expressed from either the EF1a-short (EFS) promoter or the EF1a promoter. HEK-RT1 reporter cells were stably transduced with the indicated ProCas9 variants or Cas9 WT. Genome editing activity was quantified at the indicated days post-transduction. Error bars represent the standard deviation of triplicates. FIG. 4C illustrates results of a T7 endonuclease 1 (T7E1) assay for leakiness assessment at the endogenous PCSK9 locus. HepG2 cells were stably transduced with the indicated sgRNAs and with ProCas9 variants or with Cas9 WT. Cells were selected on puromycin and harvested at day 8 post-transduction for T7 endonuclease 1 analysis. While WT Cas9 showed high levels of editing, no leakiness was observed with any of the ProCas9 constructs. FIG. 4D illustrates mutational patterns and editing efficiency at the PCSK9 locus of samples shown in FIG. 4C. Indels were quantified using Tracking of Indels by DEcomposition (TIDE). For clarity, the fraction of non-edited cells is represented as negative percentages. FIG. 4E illustrates quantification of ProCas9 leakiness, using methods like those used in FIG. 4C in A549 and HAP1 cells. Cells were selected on puromycin and harvested at day 7 post-transduction for T7 endonuclease 1 analysis. FIG. 4F illustrates quantification of ProCas9 activation in response to various control (dTEV, pCF708) or Flavivirus (ZIKV, pCF709; WNV, pCF710) proteases. ProCas9 reporter cell lines were stably transduced with the indicated protease vectors. At day 3 post-transduction, cells were treated with doxycycline to induce GFP reporter expression. Error bars represent the standard deviation of triplicates. Significance was assessed by comparing each sample to its respective deactivated tobacco etch virus (dTEV) protease control (unpaired, two-tailed t test, n=3, *p<0.05; ns, not significant). FIG. 4G illustrates genome editing activity in Flavivirus ProCas9 reporter cell lines (as in FIG. 4F), at day 4 or 8 post-transduction. FIG. 4H illustrates protease-sensitive editing at the endogenous PCSK9 locus. A T7 endonuclease 1 (T7E1) assay was performed of A549 and HAP1 Flavivirus ProCas9 cell lines (sgNT, sgPCSK9-4) stably transduced with the indicated mTagBFP2-tagged viral proteases. At day 4 post-transduction, mTagBFP2-positive cells were sorted and harvested for the T7E1 analysis. FIG. 4I illustrates ProCas9Flavi activation by Flavivirus (Flavi) proteases. The symbol * indicates the small subunit of the activated ProCas9Flavi (29 kDa). The symbol ** indicates the large subunit of the activated ProCas9Flavi (137 kDa). FIG. 4J shows an immunoblot of Cas9 in HEK293T co-transfected with plasmids expressing Cas9 WT or ProCas9Flavi, and dTEV or WNV proteases. The C-Cas9 (clone 10C11-A12) antibody recognizes the large subunit of the activated ProCas9Flavi (**137 kDa). FIG. 4K shows an immunoblot of Cas9 in HEK293T co-transfected with plasmids expressing Cas9 WT or ProCas9Flavi and dTEV or WNV proteases. The Flag-tag (clone M2) antibody recognizes the small subunit of the activated ProCas9Flavi (*29 kDa). ***, likely small-subunit-ProCas9Flavi-T2A-mCherry (55 kDa). Protein ladders indicate reference molecular weight markers.

FIG. 5A-5D illustrates that ProCas9 Enables Selective Genomically Encoded Programmable Response Systems, referred to a genomic shredding. FIG. 5A graphically illustrates CRISPR-Cas-programmed cell depletion. HEK293T and HAP1 cells expressing Cas9 WT were transduced with mCherry-tagged sgRNAs. After mixing with parental cells, the fraction of mCherry-positive cells was quantified over time. Different sgRNAs targeted a neutral gene (sgOR2B6), an essential gene (sgRPA1), greater than 100,000 genomic loci (sgCIDE), and a non-targeting control (sgNT) and the fractions of mCherry-positive cells were compared. Error bars represent the standard deviation of triplicates. FIG. 5B graphically illustrates results of a competitive proliferation assay analogous to the assay described for FIG. 5A, conducted in HEK293T and HAP1 cells expressing the ProCas9Flavi system. Note that sgCIDE-positive cells show little or no depletion because the ProCas9Flavi is in its inactive, vigilant state. FIG. 5C schematically illustrates ProCas9Flavi activation by Flavivirus proteases expressed from genomically integrated lentiviral vectors. FIG. 5D graphically illustrates depletion of protease-expressing cells by Cas9 proteins that are activated by the protease. The results shown are of a competitive proliferation assay in HEK293T ProCas9Flavi cells expressing the indicated mCherry-tagged sgRNAs or a non-targeting control (sgNT) used for normalization. Cells were partially transduced with lentiviral vectors expressing a GFP-tagged dTEV or WNV protease and cell depletion quantified by flow cytometry. Note that the WNV protease leads to protective cell death (altruistic defense) in sgCIDE-expressing cells through activation of the ProCas9Flavi system. Error bars represent the SD of triplicates. Significance was assessed by comparing each sample to its respective dTEV control (unpaired, two-tailed t test, n=3, *p<0.05; ns, not significant).

FIG. 6 schematically illustrates application of Cas9 Circular Permutants for various uses. Cas9 circular permutants (Cas9-CPs) can be used as single-molecule sensor effectors for protease tracing and molecular recording, or as optimized scaffolds for modular CP-fusion proteins with novel and enhanced functionalities.

FIG. 7 illustrates greater cell survival when essential genes are targeted than when repetitive genomic DNA is targeted by the guide RNAs and the CRISPR-Cas genome shredder. As shown, glioblastoma cells in culture are rapidly and efficiently eliminated.

FIG. 8 illustrates that CRISPR-Cas genome shredding rapidly and efficiently eliminates selected target cells in culture. As illustrated, target cell elimination is more rapid when repetitive sequences are targeted than when targeting essential genes such as the replication protein A1 (RPA1). OR2B6 was used as a non-essential gene control. HEK-pCF226 cells are cells from the human embryonic kidney HEK293T cell line that express Cas9. A549-pCF226 cells are cells from the human lung cancer A549 cell line that express Cas9. U251-pCF226 cells are cells from the human glioblastoma cell line U-251 that express Cas9.

FIG. 9A-9C illustrate targeting of glioblastoma cells for cell death with sgCIDE guide RNAs that target repetitive genomic sites. FIG. 9A is a schematic of one type of CRISPR-Cas genome shredding system. A cell line that expresses Cas9 (e.g., a glioblastoma cell line, GBM-Cas9) was transfected with an sgRNA vector expressing either a sgCIDE guide RNA (targeting repetitive genomic sites), an sgEssential gene guide RNA (targeting an essential gene), or a control sgRNA (sgNT, non-targeting). As shown in the flow cytometry graph to the right, the number of cell counts over time can be observed by the mNeonGreen expression cassette, which is a marker for cell survival. Use of the sgNT (non-targeting) guide RNA does not reduce cell numbers, and increases in the numbers of mNeonGreen-expressing cells are observed over time. Use of the sgCIDE or sgEssential gene guide RNAs can reduce the numbers of mNeonGreen-expressing cells observed over time. FIG. 9B illustrates that expression of the genome shredding guide RNAs (sgCIDE1-10, Table 2) that recognize repetitive sequences quickly destroyed U251 glioblastoma cells that expressed Cas9. In contrast, expression of the essential gene guide RNA (sgRPA1) led to substantially less cell death, and the non-targeting (sgNT control) guide RNAs had essentially no effect on cell survival. FIG. 9C illustrates that expression of the genome shredding guide RNAs (sgCIDE1-10, Table 2) that recognize repetitive sequences quickly destroyed the LN229 glioblastoma cells that expressed Cas9. As illustrated, expression of the essential gene guide RNA (sgRPA1) led to substantially less cell death, and the non-targeting (sgNT control) guide RNAs had essentially no effect on cell survival.

FIG. 10A-10F illustrate that genome shredding can target glioblastoma cells for cell death whether or not those cells are sensitive to chemotherapy. FIG. 10A graphically illustrates U251 cell viability after treatment with the chemotherapeutic agent temozolomide (TMZ). U251 glioblastoma cells are sensitive to TMZ and the viability of these cells decreases over the time of TMZ treatment. FIG. 10B graphically illustrates T98G cell viability after treatment with the chemotherapeutic agent temozolomide (TMZ). U251 glioblastoma cells are resistant to TMZ and the viability of these cells does not decrease significantly over the time of TMZ treatment. FIG. 10C graphically illustrates TMZ-sensitive U251 cell viability after treatment with a CRISPR-Cas genome shredding guide RNA (sgCIDE-1, Table 2). FIG. 10D graphically illustrates TMZ-resistant T98G cell viability after treatment with a CRISPR-Cas genome shredding guide RNA (sgCIDE-1, Table 2). FIG. 10E graphically summarizes the percentage of TMZ-sensitive U251 cells arrested in the sub-G1 stage of the cell cycle after treatment with the chemotherapeutic agent temozolomide (TMZ) or the CRISPR-Cas genome shredding guide RNAs (sgCIDE-1,-2, or -3, see Table 2). FIG. 10F graphically summarizes the percentage of TMZ-resistant T98G arrested in the sub-G1 stage of the cell cycle after treatment with the chemotherapeutic agent temozolomide (TMZ) or CRISPR-Cas genome shredding guide RNAs (sgCIDE-1, sgCIDE-2, or sgCIDE-3, see Table 2). As illustrated, TMZ is only effective against TMZ-sensitive glioblastoma cells, but the CRISPR-Cas genome shredding guide RNAs effectively kill or arrest cell growth of glioblastoma cells whether or not those cells are susceptible to chemotherapeutic agents such as TMZ.

FIG. 11A-11C illustrate that co-delivery of a single Cas9-sgCIDE expression vector significantly reduces the incidence of escape from genome shredding. FIG. 11A graphically illustrates the percentage cell depletion of the indicated U251-Cas9 genome shredding ‘escapee’ clones (sgC1, sgCIDE-1, sgC2, and sgCIDE-2) when these U251-Cas9 cells were re-transduced with the sgRNA expression vector. The cell depletion of control lines treated with a lentiviral vector (pCF820) expressing various sgCIDE or non-targeting control guide RNAs (sgNT) and an mCherry fluorescence marker are also shown. As illustrated, re-introduction of the sgCIDE expression vectors alone did not reduce cell proliferation of escapee clones. FIG. 11B schematically illustrates the process by some cells can escape genome shredding when only the sgCIDE expression vector is introduced into cells that were thought to express Cas9 (top). Use of an expression vector that expresses both Cas9 and the sgCIDE RNA (bottom) can significantly reduce the incidence of escape. FIG. 11C graphically illustrates significantly reduced cell proliferation by escapee cloned sgC1, sgCIDE-1, sgC2, and sgCIDE-2 lines when an expression vector expressing both the Cas9 and the sgCIDE is used.

FIG. 12A-12B illustrate improved “CRISPR-Safe” constructs and their utility for genome shredding. FIG. 12A is a schematic illustrating the generation and use of a CRISPR-resistant viral packaging cell line termed “CRISPR-Safe.” HEK293T cells were transduced with a lentiviral vector (pCF525-AcrIIA4) that stably expresses the anti-CRISPR protein AcrIIA4. The AcrIIA4 protein inhibits Streptococcus pyogenes Cas9. Use of the resulting CRISPR-Safe packaging cell line enables high-titer production of all-in-one Cas9-sgCIDE viral particles. FIG. 12B illustrates that use of the CRISPR-Safe viral packaging cell line rescues viral titers of all-in-one Cas9-sgCIDE vectors. Parental U251 cells (U251-pCF226-pCF821-sgNT-1 #1) and U251 cells stably expressing AcrIIA4 (pCF525-AcrIIA4 for CRISPR-Safe) were transduced with all-in-one lentiviral vectors (pCF826) expressing an mCherry-tagged Cas9 and the indicated sgRNAs. Viral particles were produced either using standard HEK293T packaging cells or the CRISPR-Safe packaging cell line (that expresses AcrIIA4). Viral titers were assessed by flow cytometry-based quantification of mCherry expression at day two post-transduction.

### DETAILED DESCRIPTION

Described herein are methods of shredding the genomes of selected cell types, for example, selected cancer cell types.

### Genomic Shredding Technology

Described herein are genomic shredding can be used to selectively deplete or eliminate selected cell types such as specific cancer cell types. For example, a guide RNA (gRNA) or single guide RNA (sgRNA) can be used to recognize to target repetitive or highly repetitive sequences in the target genome, and a Cas nuclease can act as a pair of scissors to cleave genomic DNA. As shown in the Examples, cell depletion is greater when repetitive sequences are targeted than when essential gene sequences are targeted. The specificity of targeting can be increased by use of deactivated Cas proteins that can be activated by selected proteases.

The Cas system can recognize any sequence in the genome that matches 20 bases of a gRNA. However, each gRNA also has or is adjacent to a “Protospacer Adjacent Motif” (PAM), which is invariant for each type of Cas protein, because the PAM binds directly to the Cas protein. See Doudna et al., Science 346(6213): 1077, 1258096 (2014); and Jinek et al., Science 337:816-21 (2012). Hence, the guide RNAs can have a PAM site sequence that can be bound by a Cas protein.

When the Cas system was first described for Cas9, with a “NGG” PAM site, the PAM was somewhat limiting in that it required a GG in the right orientation to the site to be targeted. Different Cas9 species have now been described with different PAM sites. See Jinek et al., Science 337:816-21 (2012); Ran et al., Nature 520:186-91 (2015); and Zetsche et al., Cell 163:759-71 (2015). In addition, mutations in the PAM recognition domain (Table 1) have increased the diversity of PAM sites for SpCas9 and SaCas9. See Kleinstiver et al., Nat Biotechnol 33:1293-1298 (2015); and Kleinstiver et al., Nature 523:481-5 (2015).

Table 1 summarizes information about PAM sites that can be used with the guide RNAs.

TABLE 1
PAM sites (SEQ ID NOs: 101-106)

| | PAM sites |
|---|---|
| SpCas9 | NGG |
| SpCas9 VRER variant | NGCG |
| SpCas9 EQR variant | NGAG |
| SpCas9 VQR variant | NGAN or NGNG |
| SaCas9 | NNGRRT |
| SaCas9, KKH variant | NNNRRT |
| FnCas2 (Cpf1) | TTN |
| DNA annotations: | |
| N = A, C, T or G | |
| R = Purine, A or G | |

Note that the guide RNAs for SpCas9 and SaCas9 cover 20 bases in the 5′direction of the PAM site, while for FnCas2 (Cpf1) the guide RNA covers 20 bases to 3′ of the PAM.

Some examples of the specific guide RNA sequences provided herein are shown below in Table 2.

TABLE 2
sgCIDE RNA Sequences

| | | SEQ |
|---|---|---|
| | | ID |
| Name | Sequence | NO: |
| sgCIDE-1 | TGTAATCCCAGCACTTTGGG | 1 |
| sgCIDE-2 | TCCCAAAGTGCTGGGATTAC | 2 |
| sgCiDE-3 | GCCTGTAATCC(AGCACTH | 3 |
| SgCIDE-4 | CGCCTGTAATCCCAGCACTT | 4 |
| sgCIDE-5 | CCTCGGCCTCCCAAAGTGCT | 5 |
| sgCIDE-6 | CCCAGCACTTTGGGAGGCCG | 6 |
| sgCIDE-7 | CTCCCAAAGTGCTGGGATTA | 7 |
| sgCIDE-8 | CTGTAATCCCAGCACTTTGG | 8 |
| sgCIDE-9 | TCCCAGCACTTTGGGAGGCC | 9 |
| sgCIDE-10 | TTCTCCTGCCTCAGCCTCCC | 10 |
| sgCIDE-21 | AGTGAGTTCCAGGACAGCCA | 11 |
| sgCIDE-22 | TTGTTCCACCTATAGGGTTG | 12 |
| sgCIDE-23 | CTTTCTCTAGCTCCTCCATT | 13 |
| sgCIDE-24 | CCCAATGGAGGAGCTAGAGA | 14 |
| sgCIDE-31 | CCATTCTGACTGGTGTGAGA | 15 |
| sgCIDE-32 | GAAGTCCTAGCCAGAGCAAT | 16 |
| sgCIDE-33 | ATTGCTCTGGCTAGGACTTC | 17 |
| sgCIDE-34 | GTCTCCCACTATTATTGTGT | 18 |
| sgCIDE-35 | TTGAATCTGTAGATTGCTTT | 19 |
| sgCIDE-36 | CCTCCCAAGTGCTGGGATTA | 20 |
| sgCIDE-41 | AAGAAAGAAAGAAAGAAAGA | 21 |
| sgCIDE-42 | GAGAGAGAGAGAGAGAGAGA | 22 |
| sgCIDE-43 | AGGAAGGAAGGAAGGAAGGA | 23 |
| sgCIDE-44 | TAGATAGATAGATAGATAGA | 24 |
| sgCIDE-45 | CACACACACACACACACACA | 25 |
| sgCIDE-46 | TGGATGGATGGATGGATGGA | 26 |
| sgCIDE-Alu | AGTAATCCCAGCACTTTGGG | 27 |
| sgCIDE-SINE-B2 | GGGCTGGAGAGATGGCTCAG | 28 |
| sgNT-1 | GGCCAAACGTGCCCTGACGG | 29 |
| sgNT-2 | GCGATGGGGGGGTGGGTAGC | 30 |
| sgNT-3 | GACGACTAGTTAGGCGTGTA | 31 |
| sgOR2B6-1 | CATTATTCTAGTGTCACGCC | 20 |
| sgOR2B6-2 | GGGTATGAAGTTTGGTGTCC | 33 |
| sgOR2B6-3 | AATGGTCAGATTGCCAAAGA | 34 |
| sgRPA1-1 | ACAAAAGTCAGATCCGTACC | 35 |
| sgRPA1-2 | TACCTGGAGCAACTCCCGAG | 36 |
| sgRPA1-3 | ACTTTCGTCAACCAGTTCTA | 37 |

The specific guide RNA sequences can also be selected from the sequences of highly amplified loci that can be present in particular types of cancer cells. Such highly amplified loci are useful for in vivo targeting of cancer cells without killing other cells. For example, the EGFR, PDGFRA, MDM2, CDK4, or combinations thereof loci can be amplified in certain glioblastomas, and sgRNA guide RNA sequences can be selected from such EGFR, PDGFRA, MDM2, and/or CDK4 sequences.

There are a number of different types of nucleases and systems that can be used for gene shredding. The nuclease employed can in some cases be any DNA binding protein can complex with a selected guide RNA and has nuclease activity. Examples of nuclease include Streptococcus pyogenes Cas (SpCas9) nucleases, Staphylococcus aureus Cas9 (SpCas9) nucleases, Francisella novicida Cas2 (FnCas2, also called dFnCpf1) nucleases, or any combination thereof. The CRISPR-Cas systems are generally the most widely used. In some cases, the nuclease is a Cas protein. The term “protein” is used with reference to the nuclease to embrace a deactivated nuclease and an active nuclease.

CRISPR-Cas systems are generally divided into two classes. The class 1 system contains types I, III and IV, and the class 2 system contains types II, V, and VI. The class 1 CRISPR-Cas system uses a complex of several Cas proteins, whereas the class 2 system only uses a single Cas protein with multiple domains. The class 2 CRISPR-Cas system is usually preferable for gene-engineering applications because of its simplicity and ease of use.

A variety of Cas proteins can be employed in the methods described herein. Three species that have been best characterized are provided as examples. The most commonly used Cas protein is a Streptococcus pyogenes Cas9, (SpCas9). More recently described forms of Cas include Staphylococcus aureus Cas9 (SaCas9) and Francisella novicida Cas2 (FnCas2, also called FnCpf1). Jinek et al., Science 337:816-21 (2012); Qi et al., Cell 152:1173-83 (2013); Ran et al., Nature 520:186-91 (2015); Zetsche et al., Cell 163:759-71 (2015).

One example of an amino acid sequence for Streptococcus pyogenes Cas9 (SpCas9) nuclease is provided below (SEQ ID NO:38).

| | |
|---|---|
| 1 | MDKKYSIGLD IGTNSVGWAV ITDEYKVPSK KFKVLGNTDR |
| 41 | HSIKKNLIGA LLFDSGETAE ATRLKRTARR RYTRRKNRIC |
| 81 | YLQEIFSNEM AKVDDSFFHR LEESFLVEED KKHERHPIFG |
| 121 | NIVDEVAYHE KYPTIYHLRK KLVDSTDKAD LRLIYLALAH |
| 161 | MIKFRGHFLI EGDLNPDNSD VDKLFIQLVQ TYNQLFEENP |
| 201 | INASGVDAKA ILSARLSKSR RLENLIAQLP GEKKNGLFGN |
| 241 | LIALSLGLTP NFKSNFDLAE DAKLQLSKDT YDDDLDNLLA |
| 281 | QIGDQYADLF LAAKNLSDAI LLSDILRVNT EITKAPLSAS |
| 321 | MIKRYDEHHQ DLTLLKALVR QQLPEKYKEI FFDQSKNGYA |
| 361 | GYIDGGASQE EFYKFIKPIL EKMDGTEELL VKLNREDLLR |
| 401 | KQRTFDNGSI PHQIHLGELH AILRRQEDFY PFLKDNREKI |
| 441 | EKILTFRIPY YVGPLARGNS RFAWMTRKSE ETITPWNFEE |
| 481 | VVDKGASAQS FIERMTNFDK NLPNEKVLPK HSLLYEYFTV |
| 521 | YNELTKVKYV TEGMRKPAFL SGEQKKAIVD LLFKTNRKVT |
| 561 | VKQLKEDYFK KIECFDSVFI SGVEDRFNAS LGTYHDLLKI |
| 601 | IKDKDFLDNE ENEDILEDIV LTLTLFEDRE MIEERLKTYA |
| 641 | HLFDDKVMKQ LKRRRYTGWG RLSRKLINGI RDKQSGKTIL |
| 681 | DFLKSDGFAN RNFMQLIHDD SLTFKEDIQK AQVSGQGDSL |
| 721 | HEHIANLAGS PAIKKGILQT VKVVDELVKV MGRHKPENIV |
| 761 | IEMARENQTT QKGQKNSRER MKRIEEGIKE LGSQILKEHP |
| 801 | VENTQLQNEK LYLYYLQNGR DMYVDQELDI NRLSDYDVDH |
| 841 | IVPQSFLKDD SIDNKVLTRS DKNRGKSDNV PSEEVVKKMK |
| 881 | NYWRQLLNAK LITQRKFDNL TKAERGGLSE LDKAGFIKRQ |
| 921 | LVETRQITKH VAQILDSRMN TKYDENDKLI REVKVITLKS |
| 961 | KLVSDFRKDF QFYKVREINN YHHAHDAYLN AVVGTALIKK |
| 1001 | YPKLESEFVY GDYKVYDVRK MIAKSEQEIG KATAKYFFYS |
| 1041 | NIMNFFKTEI TLANGEIRKR PLIETNGETG EIVWDKGRDF |
| 1081 | ATVRKVLSMP QVNIVKKTEV QTGGFSKESI LPKRNSDKLI |
| 1121 | ARKKDWDPKK YGGFDSPTVA YSVLVVAKVE KGKSKKLKSV |
| 1161 | KELLGITIME RSSFEKNPID FLEAKGYKEV KKDLIIKLPK |
| 1201 | YSLFELENGR KRMLASAGEL QKGNELALPS KYVNFLYLAS |
| 1241 | HYEKLKGSPE DNEQKQLFVE QHKHYLDEII EQISEFSKRV |
| 1281 | ILADANLDKV LSAYNKHRDK PIREQAENII HLFTLTNLGA |
| 1321 | PAAFKYFDTT IDRKRYTSTK EVLDATLIHQ SITGLYETRI |
| 1361 | DLSQLGGD |

A cDNA that encodes the Streptococcus pyogenes Cas9 (SpCas9) is provided below (SEQ ID NO:39).

| | |
|---|---|
| 1 | GACAAGAAGT ACAGCATCGG CCTGGACATC GGCACCAACT |
| 41 | CTGTGGGCTG GGCCGTGATC ACCGACGAGT ACAAGGTGCC |
| 81 | CAGCAAGAAA TTCAAGGTGC TGGGCAACAC CGACCGGCAC |
| 121 | AGCATCAAGA AGAACCTGAT CGGAGCCCTG CTGTTCGACA |
| 161 | GCGGCGAAAC AGCCGAGGCC ACCCGGCTGA AGAGAACCGC |
| 201 | CAGAAGAAGA TACACCAGAC GGAAGAACCG GATCTGCTAT |
| 241 | CTGCAAGAGA TCTTCAGCAA CGAGATGGCC AAGGTGGACG |
| 281 | ACAGCTTCTT CCACAGACTG GAAGAGTCCT TCCTGGTGGA |
| 321 | AGAGGATAAG AAGCAGGAGC GGCACCCCAT CTTCGGCAAC |
| 361 | ATCGTGGACG AGGTGGCCTA CCACGAGAAG TACCCCACCA |
| 401 | TCTACCACCT GAGAAAGAAA CTGGTGGACA GCACCGACAA |
| 441 | GGCCGACCTG CGGCTGATCT ATCTGGCCCT GGCCCACATG |
| 481 | ATCAAGTTCC GGGGCCACTT CCTGATCGAG GGCGACCTGA |
| 521 | ACCCCGACAA CAGCGACGTG GACAAGCTGT TCATCCAGCT |
| 561 | GGTGCAGACC TACAACCAGC TGTTCGAGGA AAACCCCATC |
| 601 | AACGCCAGCG GCGTGGACGC CAAGGCCATC CTGTCTGCCA |
| 641 | GACTGAGCAA GAGCAGACGG CTGGAAAATC TGATCGCCCA |
| 681 | GCTGCCCGGC GAGAAGAAGA ATGGCCTGTT CGGAAACCTG |
| 721 | ATTGCCCTGA GCCTGGGCCT GACCCCCAAC TTCAAGAGCA |
| 761 | ACTTCGACCT GGCCGAGGAT GCCAAACTGC AGCTGAGCAA |
| 801 | GGACACCTAC GAGGAGGAGC TGGACAACCT GCTGGCCCAG |
| 841 | ATCGGCGACC AGTACGCCGA CCTGTTTCTG GCCGCCAAGA |
| 881 | ACCTGTCCGA CGCCATCCTG CTGAGCGACA TCCTGAGAGT |
| 921 | GAACACCGAG ATCACCAAGG CCCCCCTGAG CGCCTCTATG |
| 961 | ATCAAGAGAT ACGACGAGCA CCACCAGGAC CTGACCCTGC |
| 1001 | TGAAAGCTCT CGTGCGGCAG CAGCTGCCTG AGAAGTACAA |
| 1041 | AGAGATTTTC TTCGACCAGA GCAAGAACGG CTACGCCGGC |
| 1081 | TACATTGACG GCGGAGCCAG CCAGGAAGAG TTCTACAAGT |
| 1121 | TCATCAAGCC CATCCTGGAA AAGATGGACG GCACCGAGGA |
| 1161 | ACTGCTCGTG AAGCTGAACA GAGAGGACCT GCTGCGGAAG |
| 1201 | CAGCGGACCT TCGACAACGG CAGCATCCCC CACCAGATCC |
| 1241 | ACCTGGGAGA GCTGCACGCC ATTCTGCGGC GGCAGGAAGA |
| 1281 | TTTTTACCCA TTCCTGAAGG ACAACCGGGA AAAGATCGAG |
| 1321 | AAGATCCTGA CCTTCCGCAT CCCCTACTAC GTGGGCCCTC |
| 1361 | TGGCCAGGGG AAACAGCAGA TTCGCCTGGA TGACCAGAAA |
| 1401 | GAGCGAGGAA ACCATGAGCC CCTGGAACTT CGAGGAAGTG |
| 1441 | GTGGACAAGG GCGCTTCCGC CCAGAGCTTC ATCGAGCGGA |
| 1481 | TGACCAACTT CGATAAGAAC CTGCCCAACG AGAAGGTGCT |
| 1521 | GCCCAAGCAC AGCCTGCTGT ACGAGTAGTT CACCGTGTAT |
| 1561 | AACGAGCTGA CCAAAGTGAA ATACGTGACC GAGGGAATGA |
| 1601 | GAAAGCCCGC CTTCCTGAGC GGCGAGCAGA AAAAGGCCAT |
| 1641 | CGTGGACCTG CTGTTCAAGA CCAACCGGAA AGTGACCGTG |
| 1681 | AAGCAGCTGA AAGAGGACTA CTTCAAGAAA ATCGAGTGCT |
| 1721 | TCGACTCCGT GGAAATCTCC GGCGTGGAAG ATCGGTTCAA |
| 1761 | CGCCTCCCTG GGCACATACC ACGATCTGCT GAAAATTATC |
| 1801 | AAGGACAAGG ACTTCCTGGA CAATGAGGAA AACGAGGACA |
| 1841 | TTCTGGAAGA TATCGTGCTG ACCCTGACAC TGTTTGAGGA |
| 1881 | CAGAGAGATG ATCGAGGAAC GGCTGAAAAC CTATGCCCAC |
| 1921 | CTGTTCGACG ACAAAGTGAT GAAGCAGCTG AAGCGGCGGA |
| 1961 | GATACACCGG CTGGGGCAGG CTGAGCCGGA AGCTGATCAA |
| 2001 | CGGCATCCGG GACAAGCAGT CCGGCAAGAC AATCCTGGAT |
| 2041 | TTCCTGAAGT CCGACGGCTT CGCCAACAGA AACTTCATGC |
| 2081 | AGCTGATCCA CGACGACAGC CTGACCTTTA AAGAGGACAT |
| 2121 | CCAGAAAGCC CAGGTGTCCG GCCAGGGCGA TAGCCTGCAC |
| 2161 | GAGCACATTG CCAATCTGGC CGGCAGCCCC GCCATTAAGA |
| 2201 | AGGGCATCCT GCAGACAGTG AAGGTGGTGG ACGAGCTCGT |
| 2241 | GAAAGTGATG GGCCGGCACA AGCCCGAGAA CATCGTGATC |
| 2281 | GAAATGGCCA GAGAGAACCA GACCACCCAG AAGGGACAGA |
| 2321 | AGAACAGCCG CGAGAGAATG AAGCGGATCG AAGAGGGCAT |
| 2361 | CAAAGAGCTG GGCAGCCAGA TCCTGAAAGA ACACCCCGTG |
| 2401 | GAAAACACCC AGCTGCAGAA CGAGAAGCTG TACCTGTACT |
| 2441 | ACCTGCAGAA TGGGCGGGAT ATGTACGTGG ACCAGGAACT |
| 2481 | GGACATCAAC CGGCTGTCCG ACTAGGATGT GGACCATATC |
| 2521 | GTGCCTCAGA GCTTTCTGAA GGACGACTCC ATCGACAACA |
| 2561 | AGGTGCTGAC CAGAAGCGAC AAGAACCGGG GCAAGAGCGA |
| 2601 | CAACGTGCCC TCCGAAGAGG TCGTGAAGAA GATGAAGAAC |
| 2641 | TACTGGCGGC AGCTGCTGAA CGCCAAGCTG ATTACCCAGA |
| 2681 | GAAAGTTCGA CAATCTGACC AAGGCCGAGA GAGGCGGCCT |
| 2721 | GAGCGAACTG GATAAGGCCG GCTTCATCAA GAGACAGCTG |
| 2761 | GTGGAAACCC GGCAGATCAC AAAGCACGTG GCACAGATCC |
| 2801 | TGGACTCCCG GATGAACACT AAGTACGACG AGAATGACAA |
| 2841 | GCTGATCCGG GAAGTGAAAG TGATCACCCT GAAGTCCAAG |
| 2881 | CTGGTGTCCG ATTTCCGGAA GGATTTCCAG TTTTACAAAG |
| 2921 | TGCGCGAGAT CAACAACTAC CACCACGCCC ACGACGCCTA |
| 2961 | CCTGAACGCC GTCGTGGGAA CCGCCCTGAT CAAAAAGTAC |
| 3001 | CCTAAGCTGG AAAGCGAGTT CGTGTACGGC GACTACAAGG |
| 3041 | TGTACGACGT GCGGAAGATG ATCGCCAAGA GCGAGCAGGA |
| 3081 | AATCGGCAAG GCTACCGCCA AGTACTTCTT CTACAGCAAC |
| 3121 | ATCATGAACT TTTTCAAGAC CGAGATTACC CTGGCCAACG |
| 3161 | GCGAGATCCG GAAGCGGCCT CTGATCGAGA CAAACGGCGA |
| 3201 | AACCGGGGAG ATCGTGTGGG ATAAGGGCCG GGATTTTGCC |
| 3241 | ACCGTGCGGA AAGTGCTGAG CATGCCCCAA ACAGGCGGCT |
| 3281 | TGAAAAAGAC CGAGGTGCAG GTGAATATCG TCAGCAAAGA |
| 3321 | GTCTATCCTG CCCAAGAGGA ACAGCGATAA GCTGATCGCC |
| 3361 | AGAAAGAAGG ACTGGGACCC TAAGAAGTAC GGCGGCTTCG |
| 3401 | ACAGCCCCAC CGTGGCCTAT TCTGTGCTGG TGGTGGCCAA |
| 3441 | AGTGGAAAAG GGCAAGTCCA AGAAACTGAA GAGTGTGAAA |
| 3481 | GAGCTGCTGG GGATCACCAT CATGGAAAGA AGCAGCTTCG |
| 3521 | AGAAGAATCC CATCGACTTT CTGGAAGCCA AGGGCTACAA |
| 3561 | AGAAGTGAAA AAGGACCTGA TCATCAAGCT GCCTAAGTAC |
| 3601 | TCCCTGTTCG AGCTGGAAAA CGGCCGGAAG AGAATGCTGG |
| 3641 | CCTCTGCCGG CGAACTGCAG AAGGGAAACG AACTGGCCCT |
| 3681 | GCCCTCCAAA TATGTGAACT TCCTGTACCT GGCCAGCCAC |
| 3721 | TATGAGAAGC TGAAGGGCTC CCCCGAGGAT AATGAGCAGA |
| 3761 | AACAGCTGTT TGTGGAACAG CACAAGCACT ACCTGGACGA |
| 3801 | GATCATCGAG CAGATCAGCG AGTTCTCCAA GAGAGTGATC |
| 3841 | CTGGCCGACG CTAATCTGGA CAAAGTGCTG TCCGCCTACA |
| 3881 | ACAAGCACCG GGATAAGCCC ATCAGAGAGC AGGCCGAGAA |
| 3921 | TATCATCCAC CTGTTTACCC TGACCAATCT GGGAGCCCCT |
| 3961 | GCCGCCTTCA AGTACTTTGA CACCACCATC GACCGGAAGA |
| 4001 | GGTACACCAG CACCAAAGAG GTGCTGGACG CCACCCTGAT |
| 4041 | CCACCAGAGC ATCACCGGCC TGTACGAGAC ACGGATCGAC |
| 4081 | CTGTCTCAGC TGGGAGGCGA C |

An amino acid sequence for a Francisella novicida Cas2 (FnCas2, also called FnCpf1) is shown below (SEQ ID NO:40).

| | |
|---|---|
| 1 | MTQFEGFTNL YQVSKTLRFE LIPQGKTLKH IQEQGFIEED |
| 41 | KARNDHYKEL KPIIDRIYKT YADQCLQLVQ LDWENLSAAI |
| 81 | DSYRKEKTEE TRNALIEEQA TYRNAIHDYF IGRTDNLTDA |
| 121 | INKRHAEIYK GLFKAELFNG KVLKQLGTVT TTEHENALLR |
| 161 | SFDKFTTYFS GFYENRKNVF SAEDISTAIP HRIVQDNFPK |
| 201 | FKENCHIFTR LITAVPSLRE HFENVKKAIG IFVSTSIEEV |
| 241 | FSFPFYNQLL TQTQIDLYNQ LLGGISREAG TEKIKGLNEV |
| 281 | LNLAIQKNDE TAHIIASLPH RFIPLFKQIL SDRNTLSFIL |
| 321 | EEFKSDEEVI QSFCKYKTLL RNENVLETAE ALFNELNSID |
| 361 | LTHIFISHKK LETISSALCD HWDTLRNALY ERRISELTGK |
| 401 | ITKSAKEKVQ RSLKHEDINL QEIISAAGKE LSEAFKQKTS |
| 441 | EILSHAHAAL DQPLPTTLKK QEEKEILKSQ LDSLLGLYHL |
| 481 | LDWFAVDESN EVDPEFSARL TGIKLEMEPS LSFYNKARNY |
| 521 | ATKKPYSVEK FKLNFQMPTL ASGWDVNKEK NNGAILFVKN |
| 561 | GLYYLGIMPK QKGRYKALSF EPTEKTSEGF DKMYYDYFPD |
| 601 | AAKMIPKCST QLKAVTAHFQ THTTPILLSN NFIEPLEITK |
| 641 | EIYDLNNPEK EPKKFQTAYA KKTGDQKGYR EALCKWIDFT |
| 681 | RDFLSKYTKT TSIDLSSLRP SSQYKDLGEY YAELNPLLYH |
| 721 | ISFQRIAEKE IMDAVETGKL YLFQIYNKDF AKGHHGKPNL |
| 761 | HTLYWTGLFS PENLAKTSIK LNGQAELFYR PKSRMKRMAH |
| 801 | RLGEKMLNKK LKDQKTPIPD TLYQELYDYV NHRLSHDLSD |
| 841 | EARALLPNVI TKEVSHEIIK DRRFTSDKFF FHVPITLNYQ |
| 881 | AANSPSKFNQ RVNAYLKEHP ETPIIGIDRG ERNLIYITVI |
| 921 | DSTGKILEQR SLNTIQQFDY QKKLDNREKE RVAARQAWSV |
| 961 | VGTIKDLKQG YLSQVIHEIV DLMIHYQAVV VLENLNFGFK |
| 1001 | SKRTGIAEKA VYQQFEKMLI DKLNCLVLKD YPAEKVGGVL |
| 1041 | NPYQLTDQFT SFAKMGTQSG FLEYVPAPYT SKIDPLTGFV |
| 1081 | DPFVWKTIKN HESRKHFLEG FDFLHYDVKT GDFILHFKMN |
| 1121 | RNLSFQRGLP GFMPAWDIVF EKNETQFDAK GTPFIAGKRI |
| 1161 | VPVIENHRFT GRYRDLYPAN ELIALLEEKG IVFRDGSNIL |
| 1201 | PKLLENDDSH AIDTMVALIR SVLQMRNSNA ATGEDYINSP |
| 1241 | VRDLNGVCFD SRFQNPEWPM DADANGAYHI ALKGQLLLNH |
| 1281 | LKESKDLKLQ NGISNQDWLA YIQELRN |

A cDNA that encodes the foregoing Francisella novicida Cas2 (FnCas2, also called dFnCpf1) polypeptide is shown below (SEQ ID NO:41).

| | |
|---|---|
| 1 | ATGACACAGT TCGAGGGCTT TACCAACCTG TATCAGGTGA |
| 41 | GCAAGACACT GCGGTTTGAG CTGATCCCAC AGGGCAAGAC |
| 81 | CCTGAAGGAC ATCCAGGAGC AGGGCTTCAT CGAGGAGGAC |
| 121 | AAGGCCCGCA ATGATCACTA CAAGGAGCTG AAGCCCATCA |
| 161 | TCGATCGGAT CTACAAGACC TATGCCGACC AGTGCCTGCA |
| 201 | GCTGGTGCAG CTGGATTGGG AGAACCTGAG CGCCGCCATC |
| 241 | GAGTCCTATA GAAAGGAGAA AACCGAGGAG ACAAGGAACG |
| 281 | CCCTGATCGA GGAGCAGGCC ACATATCGCA ATGCCATCCA |
| 321 | CGACTACTTC ATCGGCCGGA CAGACAACCT GACCGATGCC |
| 361 | ATCAATAAGA GACACGCCGA GATCTACAAG GGCCTGTTCA |
| 401 | AGGCCGAGCT GTTTAATGGC AAGGTGCTGA AGCAGCTGGG |
| 441 | CACCGTGACC ACAACCGAGC ACGAGAACGC CCTGCTGCGG |
| 481 | AGCTTCGACA AGTTTACAAC CTACTTCTCC GGCTTTTATG |
| 521 | AGAACAGGAA GAACGTGTTC AGCGCCGAGG ATATCAGCAC |
| 561 | AGCCATCCCA CACCGCATCG TGCAGGACAA CTTCCCCAAG |
| 601 | TTTAAGGAGA ATTGTCACAT CTTCACACGC CTGATCACCG |
| 721 | CCGTGCCCAG CCTGCGGGAG CACTTTGAGA ACGTGAAGAA |
| 761 | GGCCATCGGC ATCTTCGTGA GCACCTCCAT CGAGGAGGTG |
| 801 | TTTTCCTTCC CTTTTTATAA CCAGCTGCTG ACACAGACCC |
| 841 | AGATGGACCT GTATAACCAG CTGCTGGGAG GAATCTCTCG |
| 881 | GGAGGCAGGC ACCGAGAAGA TCAAGGGCCT GAACGAGGTG |
| 921 | CTGAATCTGG CCATCCAGAA GAATGATGAG ACAGCCCACA |
| 961 | TCATCGCCTC CCTGCCACAC AGATTCATCC CCCTGTTTAA |
| 1001 | GCAGATCCTG TCCGATAGGA ACACCCTGTC TTTCATCCTG |
| 1041 | GAGGAGTTTA AGAGCGACGA GGAAGTGATC CAGTCCTTCT |
| 1081 | GCAAGTACAA GACACTGCTG AGAAACGAGA ACGTGCTGGA |
| 1121 | GACAGCCGAG GCCCTGTTTA ACGAGCTGAA CAGCATCGAC |
| 1161 | CTGAGACACA TCTTCATCAG CCACAAGAAG CTGGAGACAA |
| 1201 | TCAGCAGCGC CCTGTGCGAC CACTGGGATA CACTGAGGAA |
| 1241 | TGCCCTGTAT GAGCGGAGAA TCTCCGAGCT GACAGGCAAG |
| 1281 | ATCACCAAGT CTGCCAAGGA GAAGGTGCAG CGCAGCCTGA |
| 1321 | AGCACGAGGA TATCAACCTG CAGGAGATCA TCTCTGCCGC |
| 1361 | AGGCAAGGAG CTGAGCGAGG CCTTCAAGCA GAAAACCAGC |
| 1401 | GAGATCCTGT CCCACGCACA CGCCGCCCTG GATCAGCCAC |
| 1441 | TGCCTACAAC CCTGAAGAAG CAGGAGGAGA AGGAGATCCT |
| 1481 | GAAGTCTCAG CTGGACAGCC TGCTGGGCCT GTACCACCTG |
| 1521 | CTGGACTGGT TTGCCGTGGA TGAGTCCAAC GAGGTGGACC |
| 1561 | CCGAGTTCTC TGCCCGGCTG ACCGGCATCA AGCTGGAGAT |
| 1601 | GGAGCCTTCT CTGAGCTTCT ACAACAAGGC CAGAAATTAT |
| 1641 | GCCACCAAGA AGCCCTACTC CGTGGAGAAG TTCAAGCTGA |
| 1681 | ACTTTCAGAT GCCTACACTG GCCTCTGGCT GGGACGTGAA |
| 1721 | TAAGGAGAAG AACAATGGCG CCATCCTGTT TGTGAAGAAC |
| 1761 | GGCCTGTACT ATCTGGGCAT CATGCCAAAG CAGAAGGGCA |
| 1801 | GGTATAAGGC CCTGAGCTTC GAGCCCACAG AGAAAACCAG |
| 1841 | CGAGGGCTTT GATAAGATGT ACTATGACTA CTTCCCTGAT |
| 1881 | GCCGCCAAGA TGATCCCAAA GTGCAGCACC CAGCTGAAGG |
| 1921 | CCGTGACAGC CCACTTTCAG ACCCACACAA CCCCCATCCT |
| 1961 | GCTGTCCAAC AATTTCATCG AGCCTCTGGA GATCACAAAG |
| 2001 | GAGATCTACG ACCTGAACAA TCCTGAGAAG GAGCCAAAGA |
| 2041 | AGTTTCAGAC AGCCTACGCC AAGAAAACCG GCGACCAGAA |
| 2081 | GGGCTACAGA GAGGCCCTGT GCAAGTGGAT CGACTTCACA |
| 2121 | AGGGATTTTC TGTCCAAGTA TACCAAGACA ACCTCTATCG |
| 2161 | ATCTGTCTAG CCTGCGGCCA TCCTCTCAGT ATAAGGACCT |
| 2201 | GGGCGAGTAC TATGCCGAGC TGAATCCCCT GCTGTACCAC |
| 2241 | ATCAGCTTCC AGAGAATCGC GGAGAAGGAG ATCATGGATG |
| 2281 | CCGTGGAGAC AGGCAAGCTG TACCTGTTCC AGATCTATAA |
| 2321 | CAAGGACTTT GCCAAGGGCC ACCACGGCAA GCCTAATCTG |
| 2361 | CACACACTGT ATTGGACCGG CCTGTTTTCT CCAGAGAACC |
| 2401 | TGGCCAAGAC AAGCATCAAG CTGAATGGCC AGGCCGAGCT |
| 2441 | GTTCTACCGC CCTAAGTCCA GGATGAAGAG GATGGCACAC |
| 2481 | CGGCTGGGAG AGAAGATGCT GAACAAGAAG CTGAAGGATC |
| 2521 | AGAAAACCCC AATCCCCGAC ACCCTGTACC AGGAGCTGTA |
| 2561 | CGACTATGTG AATCACAGAC TGTCCCACGA CCTGTCTGAT |
| 2601 | GAGGCCAGGG CCCTGCTGCC CAACGTGATC ACCAAGGAGG |
| 2641 | TGTCTCACGA GATCATCAAG GATAGGCGCT TTACCAGCGA |
| 2681 | CAAGTTCTTT TTCCACGTGC CTATCACACT GAACTATCAG |
| 2721 | GCCGCCAATT CCCCATCTAA GTTCAACCAG AGGGTGAATG |
| 2761 | CCTACCTGAA GGAGCACCCC GAGACACCTA TCATCGGCAT |
| 2801 | CGATCGGGGC GAGAGAAACC TGATCTATAT CACAGTGATC |
| 2841 | GCCTCCACCG GCAAGATCCT GGAGCAGCGG AGCCTGAACA |
| 2881 | CCATCCAGCA GTTTGATTAC CAGAAGAAGC TGGACAACAG |
| 2921 | GGAGAAGGAG AGGGTGGCAG CAAGGCAGGC CTGGTCTGTG |
| 2961 | GTGGGCACAA TCAAGGATCT GAAGCAGGGC TATCTGAGCC |
| 3001 | AGGTCATCCA CGAGATCGTG GACCTGATGA TCCACTACCA |
| 3041 | GGCCGTGGTG GTGCTGGAGA ACCTGAATTT CGGCTTTAAG |
| 3081 | AGCAAGAGGA CCGGCATCGC CGCGAAGGCC GTGTACCAGC |
| 3121 | AGTTCGAGAA GATGCTGATC GATAAGCTGA ATTGCCTGGT |
| 3161 | GGTGAAGGAC TATCCAGCAG AGAAAGTGGG AGGCGTGCTG |
| 3201 | AACCCATACC AGCTGACAGA CCAGTTCACC TCCTTTGCCA |
| 3241 | AGATGGGCAC CCAGTCTGGC TTCCTGTTTT ACGTGCCTGC |
| 3281 | CCCATATACA TCTAAGATCG ATCCCCTGAC CGGCTTCGTG |
| 3321 | GACCCCTTCG TGTGGAAAAC CATCAAGAAT CACGAGAGCC |
| 3361 | GCAAGCACTT CCTGGAGGGC TTCGACTTTC TGCACTACGA |
| 3401 | CGTGAAAACC GGCGACTTCA TCCTGCACTT TAAGATGAAC |
| 3441 | AGAAATCTGT CCTTCCAGAG GGGCCTGCCC GGCTTTATGC |
| 3481 | CTGCATGGGA TATCGTGTTC GAGAAGAACG AGACACAGTT |
| 3521 | TGACGCCAAG GGCACCCCTT TCATCGCCGG CAAGAGAATC |
| 3561 | GTGCCAGTGA TCGAGAATCA CAGATTCACC GGCAGATACC |
| 3601 | GGGACCTGTA TCCTGCCAAC GAGCTGATCG CCCTGCTGGA |
| 3641 | GGAGAAGGGC ATCGTGTTCA GGGATGGCTC CAACATCCTG |
| 3681 | CCAAAGCTGC TGGAGAATGA CGATTCTCAC GCCATCGACA |
| 3721 | CCATGGTGGC CCTGATCCGC AGCGTGCTGC AGATGCGGAA |
| 3761 | CTCCAATGCC GCCACAGGCG AGGACTATAT CAACAGCCCC |
| 3801 | GTGCGCGATC TGAATGGCGT GTGCTTCGAC TCCCGGTTTC |
| 3841 | AGAACCCAGA GTGGCCCATG GACGCCGATG CCAATGGCGC |
| 3881 | CTACCACATC GCCCTGAAGG GCCAGCTGCT GCTGAATCAC |
| 3921 | CTGAAGGAGA GCAAGGATCT GAAGCTGCAG AACGGCATCT |
| 3961 | CCAATCAGGA CTGGCTGGCC TACATCCAGG AGCTGCGCAA |
| 4001 | C |

The Cas proteins can be modified to improve their utility. For example, one Cas protein that can be used is the SpyCas9 amino acid sequence with a nuclear localization sequence (pCF823 vector; Streptococcus pyogenes Cas9-NLS) shown below as SEQ ID NO:42.

| | |
|---|---|
| 1 | MDKKYSIGLD IGTNSVGWAV ITDEYKVPSK KFKVLGNTDR |
| 41 | HSIKKNLIGA LLFDSGETAE ATRLKRTARR RYTRRKNRIC |
| 81 | YLQEIFSNEM AKVDDSFFHR LEESFLVEED KKHERHPIFG |
| 121 | NIVDEVAYHE KYPTIYHLRK KLVDSTDKAD LRLIYLALAH |
| 161 | MIKFRGHFLI EGDLNPDNSD VDKLFIQLVQ TYNQLFEENP |
| 201 | INASGVDAKA ILSARLSKSR RLENLIAQLP GEKKNGLFGN |
| 241 | LIALSLGLTP NFKSNFDLAE DAKLQLSKDT YDDDLDNLLA |
| 281 | QIGDQYADLF LAAKNLSDAI LLSDILRVNT EITKAPLSAS |
| 321 | MIKRYDEHHQ DLTLLKALVR QQLPEKYKEI FFDQSKNGYA |
| 361 | GYIDGGASQE EFYKFIKPIL EKMDGTEELL VKLNREDLLR |
| 401 | KQRTFDNGSI PHQIHLGELH AILRRQEDFY PFLKDNREKI |
| 441 | EKILTFRIPY YVGPLARGNS RFAWMTRKSE ETITPWNFEE |
| 481 | VVDKGASAQS FIERMTNFDK NLPNEKVLPK HSLLYEYFTV |
| 521 | YNELTKVKYV TEGMRKPAFL SGEQKKAIVD LLFKTNRKVT |
| 561 | VKQLKEDYFK KIECFDSVEI SGVEDRFNAS LGTYHDLLKI |
| 601 | IKDKDFLDNE ENEDILEDIV LTLTLFEDRE MIEERLKTYA |
| 641 | HLFDDKVMKQ LKRRRYTGWG RLSRKLINGI RDKQSGKTIL |
| 681 | DFLKSDGFAN RNFMQLIHDD SLTFKEDIQK AQVSGQGDSL |
| 721 | HEHIANLAGS PAIKKGILQT VKVVDELVKV MGRHKPENIV |
| 761 | IEMARENQTT QKGQKNSRER MKRIEEGIKE LGSQILKEHP |
| 801 | VENTQLQNEK LYLYYLQNGR DMYVDQELDI NRLSDYDVDH |
| 841 | IVPQSFLKDD SIDNKVLTRS DKNRGKSDNV PSEEVVKKMK |
| 881 | NYWRQLLNAK LITQRKFDNL TKAERGGLSE LDKAGFIKRQ |
| 921 | LVETRQITKH VAQILDSRMN TKYDENDKLI REVKVITLKS |
| 961 | KLVSDFRKDF QFYKVREINN YHHAHDAYLN AVVGTALIKK |
| 1001 | YPKLESEFVY GDYKVYDVRK MIAKSEQEIG KATAKYFFYS |
| 1041 | NIMNFFKTEI TLANGEIRKR PLIETNGETG EIVWDKGRDF |
| 1081 | ATVRKVLSMP QVNIVKKTEV QTGGFSKESI LPKRNSDKLI |
| 1121 | ARKKDWDPKK YGGFDSPTVA YSVLVVAKVE KGKSKKLKSV |
| 1161 | KELLGITIME RSSFEKNPID FLEAKGYKEV KKDLIIKLPK |
| 1201 | YSLFELENGR KRMLASAGEL QKGNELALPS KYVNFLYLAS |
| 1241 | HYEKLKGSPE DNEQKQLFVE QHKHYLDEII EQISEFSKRV |
| 1281 | ILADANLDKV LSAYNKHRDK PIREQAENII HLFTLTNLGA |
| 1321 | PAAFKYFDTT IDRKRYTSTK EVLDATLIHQ SITGLYETRI |
| 1361 | DLSQLGGD |

Another Cas protein that can be used is the SauCas9 amino acid sequence with a nuclear localization sequence (pCF825 vector; NLS-Staphylococcus aureus Cas9-NLS) shown below as SEQ ID NO:43.

| | |
|---|---|
| 1 | MAPKKKRKVG IHGVPAAKRN YILGLDIGIT SVGYGIIDYE |
| 41 | TRDVIDAGVR LFKEANVENN EGRRSKRGAR RLKRRRRHRI |
| 81 | QRVKKLLFDY NLLTDHSELS GINPYEARVK GLSQKLSEEE |
| 121 | FSAALLHLAK RRGVHNVNEV EEDTGNELST KEQISRNSKA |
| 161 | LEEKYVAELQ LERLKKDGEV RGSINRFKTS DYVKEAKQLL |
| 201 | KVQKAYHQLD QSFIDTYIDL LETRRTYYEG PGEGSPFGWK |
| 241 | DIKEWYEMLM GHCTYFPEEL RSVKYAYNAD LYNALNDLNN |
| 281 | LVITRDENEK LEYYEKFQII ENVFKQKKKP TLKQIAKEIL |
| 321 | VNEEDIKGYR VTSTGKPEFT NLKVYHDIKD ITARKEIIEN |
| 361 | AELLDQIAKI LTIYQSSEDI QEELTNLNSE LTQEEIEQIS |
| 401 | NLKGYTGTHN LSLKAINLIL DELWHTNDNQ IAIFNRLKLV |
| 441 | PKKVDLSQQK EIPTTLVDDF ILSPVVKRSF IQSIKVINAI |
| 481 | IKKYGLPNDI IIELAREKNS KDAQKMINEM QKRNRQTNER |
| 521 | IEEIIRTTGK ENAKYLIEKI KLHDMQEGKC LYSLEAIPLE |
| 561 | DLLNNPFNYE VDHIIPRSVS FDNSFNNKVL VKQEENSKKG |
| 601 | NRTPFQYLSS SDSKISYETF KKHILNLAKG KGRISKTKKE |
| 641 | YLLEERDINR FSVQKDFINR NLVDTRYATR GLMNLLRSYF |
| 681 | RVNNLDVKVK SINGGFTSFL RRKWKFKKER NKGYKHHAED |
| 721 | ALIIANADFI FKEWKKLDKA KKVMENQMFE EKQAESMPEI |
| 761 | ETEQEYKEIF ITPHQIKHIK DFKDYKYSHR VDKKPNRELI |
| 801 | NDTLYSTRKD DKGNTLIVNN LNGLYDKDND KLKKLINKSP |
| 841 | EKLLMYHHDP QTYQKLKLIM EQYGDEKNPL YKYYEETGNY |
| 881 | LTKYSKKDNG PVIKKIKYYG NKLNAHLDIT DDYPNSRNKV |
| 921 | VKLSLKPYRF DVYLDNGVYK FVTVKNLDVI KKENYYEVNS |
| 961 | KCYEEAKKLK KISNQAEFIA SFYNNDLIKI NGELYRVIGV |
| 1001 | NNDLLNRIEV NMIDITYREY LENMNDKRPP RIIKTIASKT |
| 1041 | QSIKKYSTDI LGNLYEVKSK KHPQIIKKGK RPAATKKAGQ |
| 1081 | AKKKK |

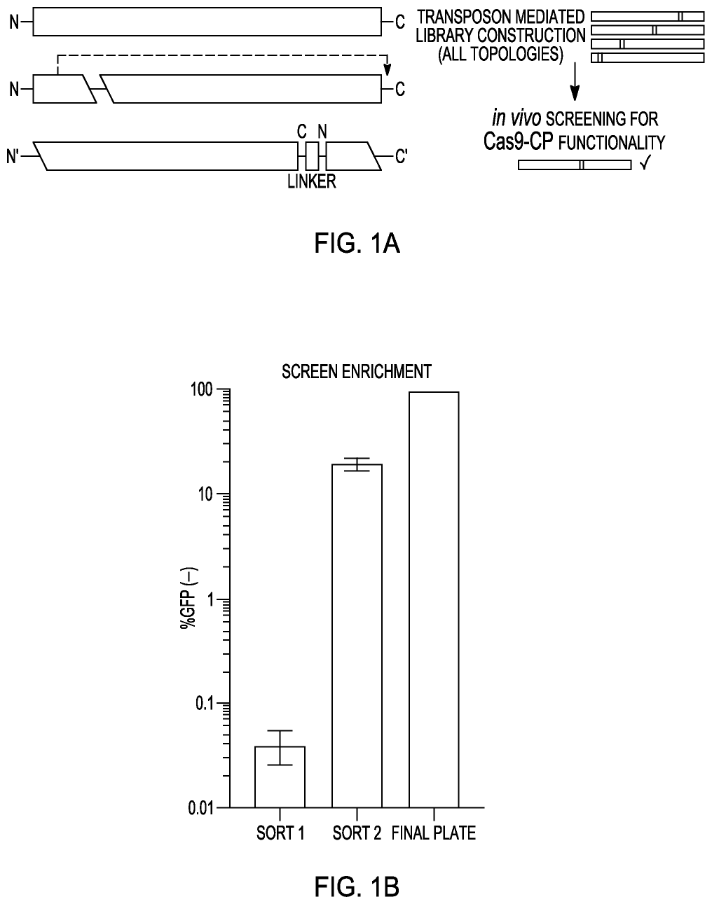

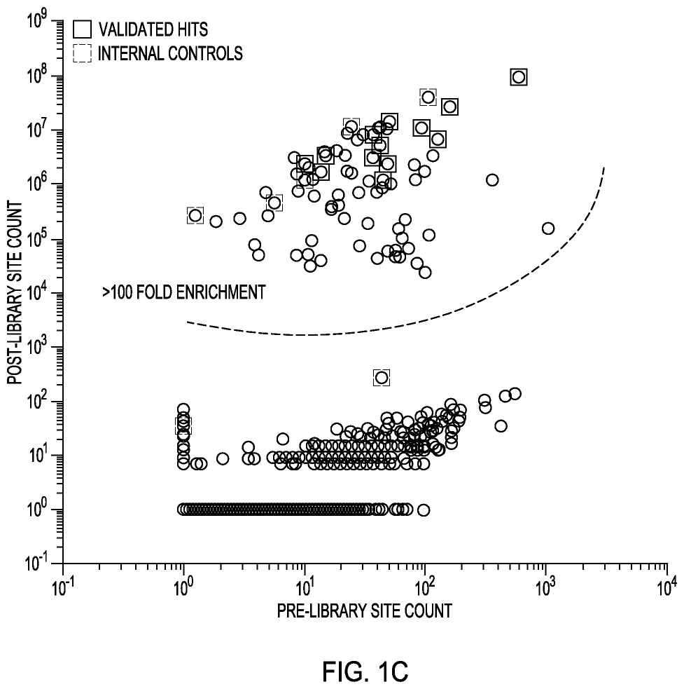

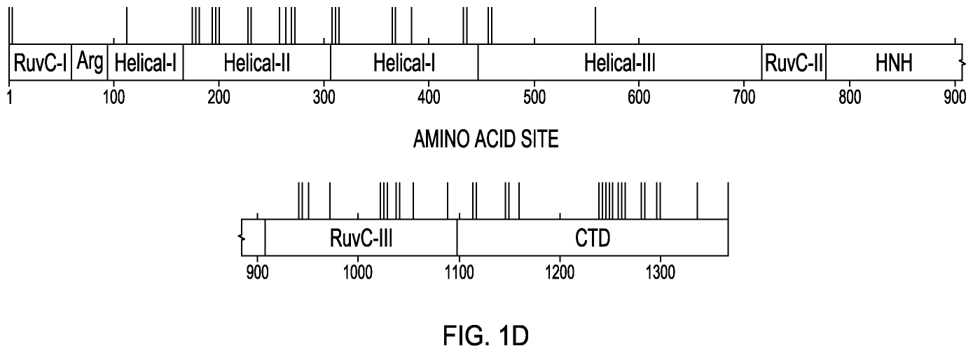

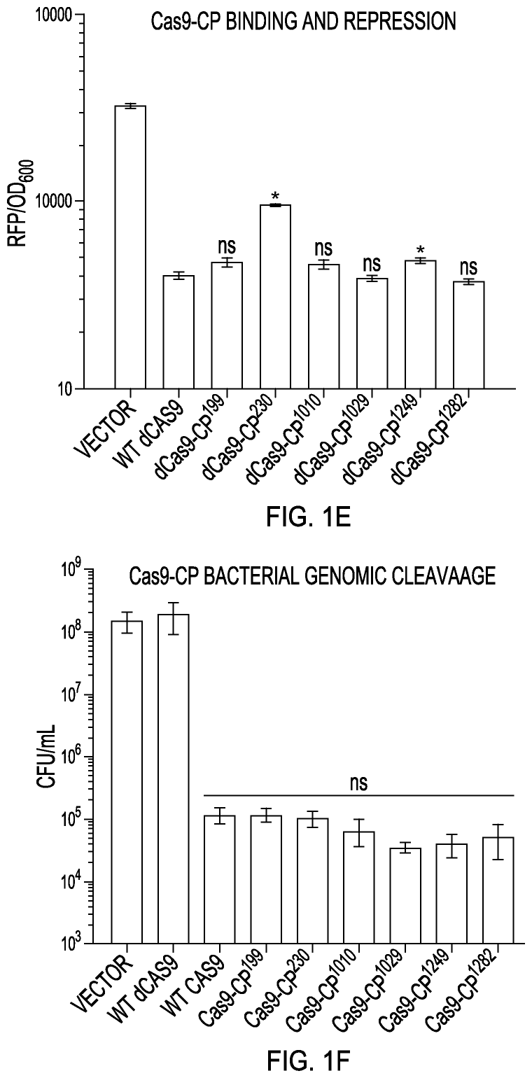

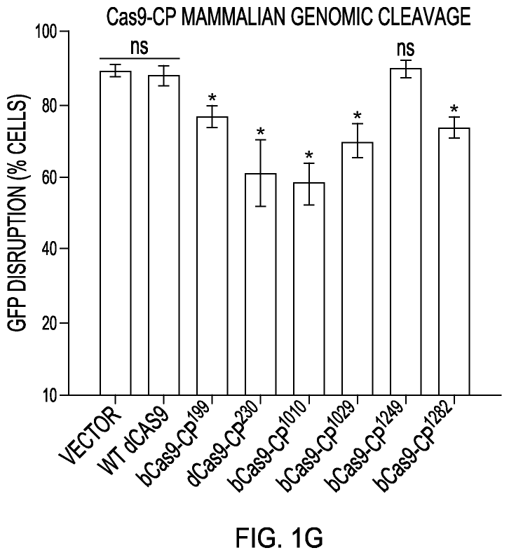

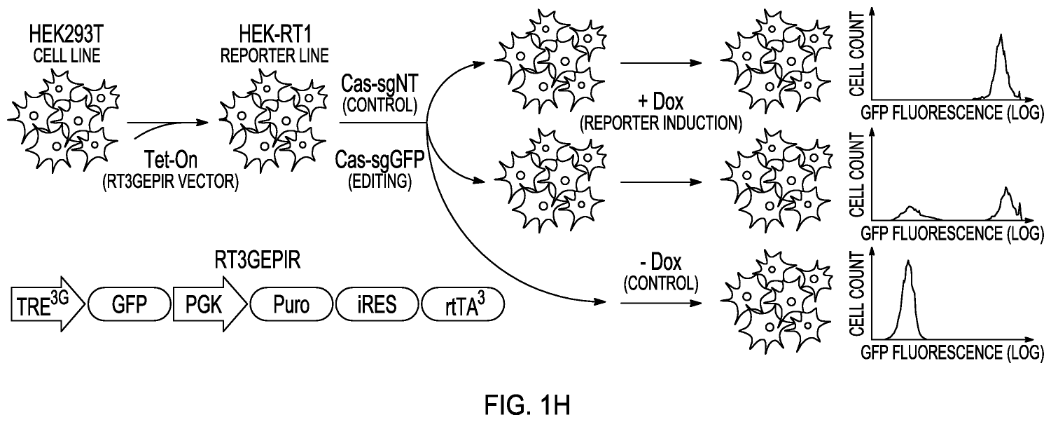

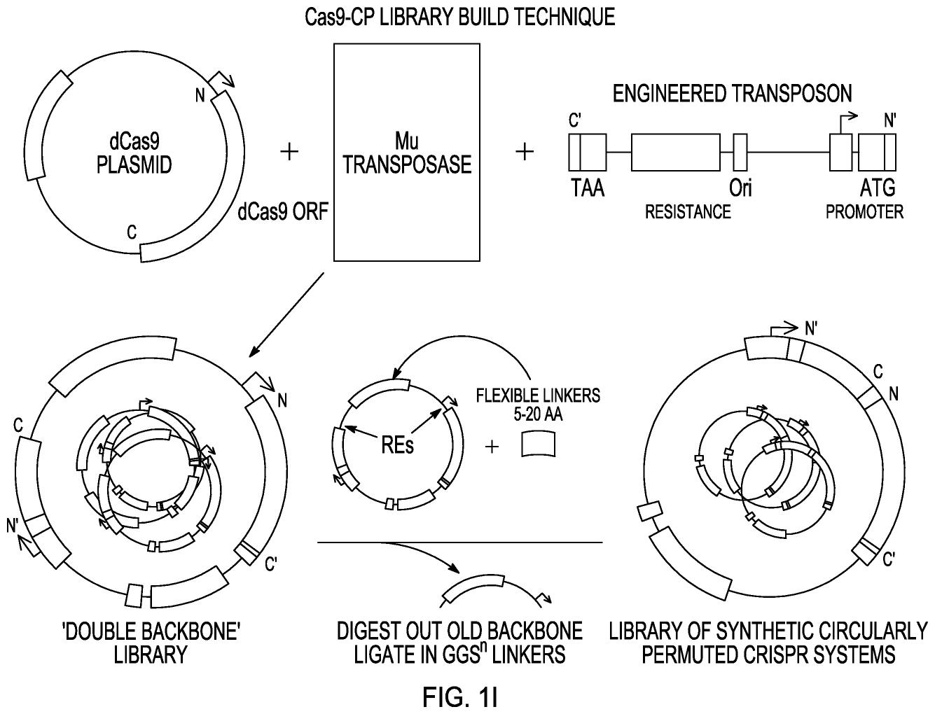

In some cases, the Cas protein is circularly permuted. Circularly permutation involves removal and in-frame fusion of a N-terminal portion of a selected Cas protein downstream of the selected Cas protein's C-terminus (as is shown in FIG. 1A). In other words, the circularly permuted Cas protein can have the same number and type of amino acids as the original, non-circularly permuted protein, but one segment is shifted from the N-terminus to the C-terminus. In some cases, there is a linker joining the shifted N-terminal segment to the original C-terminus. The linker can be cleavable by a protease so that upon cleavage the Cas protein folds properly and is a functional Cas protein.

For example, one circularly permuted Cas protein that can be used is the Cas9-CP-199 circular permutant amino acid sequence (CP2, NLS-Cas9-CP-199-NLS, QLFEE|NPINA) shown below as SEQ ID NO:44.

| | |
|---|---|
| 1 | MAPKKKRKVS ANPINASGVD AKAILSARLS KSRRLENLIA |
| 41 | QLPGEKKNGL FGNLIALSLG LTPNFKSNFD LAEDAKLQLS |
| 81 | KDTYDDDLDN LLAQIGDQYA DLFLAAKNLS DAILLSDILR |
| 121 | VNTEITKAPL SASMIKRYDE HHQDLTLLKA LVRQQLPEKY |
| 161 | KEIFFDQSKN GYAGYIDGGA SQEEFYKFIK PILEKMDGTE |
| 201 | ELLVKLNRED LLRKQRTFDN GSIPHQIHLG ELHAILRRQE |
| 241 | DFYPFLKDNR EKIEKILTFR IPYYVGPLAR GNSRFAWMTR |
| 281 | KSEETITPWN FEEVVDKGAS AQSFIERMTN FDKNLPNEKV |
| 321 | LPKHSLLYEY FTVYNELTKV KYVTEGMRKP AFLSGEQKKA |
| 361 | IVDLLFKTNR KVTVKQLKED YFKKIECFDS VEISGVEDRF |
| 401 | NASLGTYHDL LKIIKDKDFL DNEENEDILE DIVLTLTLFE |
| 441 | DREMIEERLK TYAHLFDDKV MKQLKRRRYT GWGRLSRKLI |
| 481 | NGIRDKQSGK TILDFLKSDG FANRNFMQLI HDDSLTFKED |
| 521 | IQKAQVSGQG DSLHEHIANL AGSPAIKKGI LQTVKVVDEL |
| 561 | VKVMGRHKPE NIVIEMAREN QTTQKGQKNS RERMKRIEEG |
| 601 | IKELGSQILK EHPVENTQLQ NEKLYLYYLQ NGRDMYVDQE |
| 641 | LDINRLSDYD VDAIVPQSFL KDDSIDNKVL TRSDKNRGKS |
| 681 | DNVPSEEVVK KMKNYWRQLL NAKLITQRKF DNLTKAERGG |
| 721 | LSELDKAGFI KRQLVETRQI TKHVAQILDS RMNTKYDEND |
| 761 | KLIREVKVIT LKSKLVSDFR KDFQFYKVRE INNYHHAHDA |
| 801 | YLNAVVGTAL IKKYPKLESE FVYGDYKVYD VRKMIAKSEQ |
| 841 | EIGKATAKYF FYSNIMNFFK TEITLANGEI RKRPLIETNG |
| 881 | ETGEIVWDKG RDFATVRKVL SMPQVNIVKK TEVQTGGFSK |
| 921 | ESILPKRNSD KLIARKKDWD PKKYGGFDSP TVAYSVLVVA |
| 961 | KVEKGKSKKL KSVKELLGIT IMERSSFEKN PIDFLEAKGY |
| 1001 | KEVKKDLIIK LPKYSLFELE NGRKRMLASA GELQKGNELA |
| 1041 | LPSKYVNFLY LASHYEKLKG SPEDNEQKQL FVEQHKHYLD |
| 1081 | EIIEQISEFS KRVILADANL DKVLSAYNKH RDKPIREQAE |
| 1121 | NIIHLFTLTN LGAPAAFKYF DTTIDRKRYT STKEVLDATL |
| 1161 | IHQSITGLYE TRIDLSQLGG DGGSGGSGGS GGSGGSGGSG |
| 1201 | GMDKKYSIGL DIGTNSVGWA VITDEYKVPS KKFKVLGNTD |
| 1241 | RHSIKKNLIG ALLFDSGETA EATRLKRTAR RRYTRRKNRI |
| 1281 | CYLQEIFSNE MAKVDDSFFH RLEESFLVEE DKKHERHPIF |
| 1321 | GNIVDEVAYH EKYPTIYHLR KKLVDSTDKA DLRLIYLALA |
| 1361 | HMIKFRGHFL IEGDLNPDNS DVDKLFIQLV QTYNQLFEEN |
| 1401 | PTSPKKKRKV* |

As shown, the original N-terminal amino acids (MDKK) are now at position 1202 of the SEQ ID NO:44 Cas9-CP-199 circular permutant.

Another Cas protein that can be used is the Cas9-CP-230 circular permutant amino acid sequence (CP3, NLS-Cas9-CP-230-NLS, cleavage at LIAQL|PGEKK) shown below as SEQ ID NO:45.

| | |
|---|---|
| 1 | MAPKKKRKVS ATGEKKNGLF GNLIALSLGL TPNFKSNFDL |
| 41 | AEDAKLQLSK DTYDDDLDNL LAQIGDQYAD LFLAAKNLSD |
| 81 | AILLSDILRV NTEITKAPLS ASMIKRYDEH HQDLTLLKAL |
| 121 | VRQQLPEKYK EIFFDQSKNG YAGYIDGGAS QEEFYKFIKP |
| 161 | ILEKMDGTEE LLVKLNREDL LRKQRTFDNG SIPHQIHLGE |
| 201 | LHAILRRQED FYPFLKDNRE KIEKILTFRI PYYVGPLARG |
| 241 | NSRFAWMTRK SEETITPWNF EEVVDKGASA QSFIERMTNF |
| 281 | DKNLPNEKVL PKHSLLYEYF TVYNELTKVK YVTEGMRKPA |
| 321 | FLSGEQKKAI VDLLFKTNRK VTVKQLKEDY FKKIECFDSV |
| 361 | EISGVEDRFN ASLGTYHDLL KIIKDKDFLD NEENEDILED |
| 401 | IVLTLTLFED REMIEERLKT YAHLFDDKVM KQLKRRRYTG |
| 441 | WGRLSRKLIN GIRDKQSGKT ILDFLKSDGF ANRNFMQLIH |
| 481 | DDSLTFKEDI QKAQVSGQGD SLHEHIANLA GSPAIKKGIL |
| 521 | QTVKVVDELV KVMGRHKPEN IVIEMARENQ TTQKGQKNSR |
| 561 | ERMKRIEEGI KELGSQILKE HPVENTQLQN EKLYLYYLQN |
| 601 | GRDMYVDQEL DINRLSDYDV DAIVPQSFLK DDSIDNKVLT |
| 641 | RSDKNRGKSD NVPSEEVVKK MKNYWRQLLN AKLITQRKFD |
| 681 | NLTKAERGGL SELDKAGFIK RQLVETRQIT KHVAQILDSR |
| 721 | MNTKYDENDK LIREVKVITL KSKLVSDFRK DFQFYKVREI |
| 761 | NNYHHAHDAY LNAVVGTALI KKYPKLESEF VYGDYKVYDV |
| 801 | RKMIAKSEQE IGKATAKYFF YSNIMNFFKT EITLANGEIR |
| 841 | KRPLIETNGE TGEIVWDKGR DFATVRKVLS MPQVNIVKKT |
| 881 | EVQTGGFSKE SILPKRNSDK LIARKKDWDP KKYGGFDSPT |
| 921 | VAYSVLVVAK VEKGKSKKLK SVKELLGITI MERSSFEKNP |
| 961 | IDFLEAKGYK EVKKDLIIKL PKYSLFELEN GRKRMLASAG |
| 1001 | ELQKGNELAL PSKYVNFLYL ASHYEKLKGS PEDNEQKQLF |
| 1041 | VEQHKHYLDE IIEQISEFSK RVILADANLD KVLSAYNKHR |
| 1081 | DKPIREQAEN IIHLFTLTNL GAPAAFKYFD TTIDRKRYTS |
| 1121 | TKEVLDATLI HQSITGLYET RIDLSQLGGD GGSGGSGGSG |
| 1161 | GSGGSGGSGG MDKKYSIGLA IGTNSVGWAV ITDEYKVPSK |
| 1201 | KFKVLGNTDR HSIKKNLIGA LLFDSGETAE ATRLKRTARR |
| 1241 | RYTRRKNRIC YLQEIFSNEM AKVDDSEFHR LEESFLVEED |
| 1281 | KKHERHPIFG NIVDEVAYHE KYPTIYHLRK KLVDSTDKAD |
| 1321 | LRLIYLALAH MIKFRGHFLI EGDLNPDNSD VDKLFIQLVQ |
| 1361 | TYNQLFEENP INASGVDAKA ILSARLSKSR RLENLIAQLP |
| 1401 | GTSPKKKRKV* |

Another Cas protein that can be used is the Cas9-CP-1010 circular permutant amino acid sequence (CP6, NLS-Cas9-CP-1010-NLS, cleavage at ESEFV|YGDYK) shown below as SEQ ID NO:46.

| | |
|---|---|
| 1 | MAPKKKRKVS ANGDYKVYDV RKMIAKSEQE IGKATAKYFF |
| 41 | YSNIMNFFKT EITLANGEIR KRPLIETNGE TGEIVWDKGR |
| 81 | DFATVRKVLS MPQVNIVKKT EVQTGGFSKE SILPKRNSDK |
| 121 | LIARKKDWDP KKYGGFDSPT VAYSVLVVAK VEKGKSKKLK |
| 161 | SVKELLGITI MERSSFEKNP IDFLEAKGYK EVKKDLIIKL |
| 201 | PKYSLFELEN GRKRMLASAG ELQKGNELAL PSKYVNFLYL |
| 241 | ASHYEKLKGS PEDNEQKQLF VEQHKHYLDE IIEQISEFSK |
| 281 | RVILADANLD KVLSAYNKHR DKPIREQAEN IIHLFTLTNL |
| 321 | GAPAAFKYFD TTIDRKRYTS TKEVLDATLI HQSITGLYET |
| 361 | RIDLSQLGGD GGSGGSGGSG GSGGSGGSGG MDKKYSIGLA |
| 401 | IGTNSVGWAV ITDEYKVPSK KFKVLGNTDR HSIKKNLIGA |
| 441 | LLEDSGETAE ATRLKRTARR RYTRRKNRIC YLQEIFSNEM |
| 481 | AKVDDSFFHR LEESFLVEED KKHERHPIFG NIVDEVAYHE |
| 521 | KYPTIYHLRK KLVDSTDKAD LRLIYLALAH MIKFRGHFLI |
| 561 | EGDLNPDNSD VDKLFIQLVQ TYNQLFEENP INASGVDAKA |
| 601 | ILSARLSKSR RLENLIAQLP GEKKNGLFGN LIALSLGLTP |
| 641 | NFKSNFDLAE DAKLQLSKDT YDDDLDNLLA QIGDQYADLF |
| 681 | LAAKNLSDAI LLSDILRVNT EITKAPLSAS MIKRYDEHHQ |
| 721 | DLTLLKALVR QQLPEKYKEI FFDQSKNGYA GYIDGGASQE |
| 761 | EFYKFIKPIL EKMDGTEELL VKLNREDLLR KQRTFDNGSI |
| 801 | PHQIHLGELH AILRRQEDFY PFLKDNREKI EKILTFRIPY |
| 841 | YVGPLARGNS RFAWMTRKSE ETITPWNFEE VVDKGASAQS |
| 881 | FIERMTNFDK NLPNEKVLPK HSLLYEYFTV YNELTKVKYV |
| 921 | TEGMRKPAFL SGEQKKAIVD LLFKTNRKVT VKQLKEDYFK |
| 961 | KIECFDSVEI SGVEDRFNAS LGTYHDLLKI IKDKDFLDNE |
| 1001 | ENEDILEDIV LTLTLFEDRE MIEERLKTYA HLFDDKVMKQ |
| 1041 | LKRRRYTGWG RLSRKLINGI RDKQSGKTIL DFLKSDGFAN |
| 1081 | RNFMQLIHDD SLTFKEDIQK AQVSGQGDSL HEHIANLAGS |
| 1121 | PAIKKGILQT VKVVDELVKV MGRHKPENIV IEMARENQTT |
| 1161 | QKGQKNSRER MKRIEEGIKE LGSQILKEHP VENTQLQNEK |
| 1201 | LYLYYLQNGR DMYVDQELDI NRLSDYDVDA IVPQSFLKDD |
| 1241 | SIDNKVLTRS DKNRGKSDNV PSEEVVKKMK NYWRQLLNAK |
| 1281 | LITQRKFDNL TKAERGGLSE LDKAGFIKRQ LVETRQITKH |
| 1321 | VAQILDSRMN TKYDENDKLI REVKVITLKS KLVSDFRKDF |
| 1361 | QFYKVREINN YHHAHDAYLN AVVGTALIKK YPKLESEFVY |
| 1401 | GTSPKKKRKV |

Another Cas protein that can be used is the Cas9-CP-1029 circular permutant amino acid sequence (CP9, NLS-Cas9-CP-1029-NLS, cleavage at KSEQE|IGKAT) shown below as SEQ ID NO:47.

| | |
|---|---|
| 1 | MAPKKKRKVS AKIGKATAKY FFYSNIMNFF KTEITLANGE |
| 41 | IRKRPLIETN GETGEIVWDK GRDFATVRKV LSMPQVNIVK |
| 81 | KTEVQTGGFS KESILPKRNS DKLIARKKDW DPKKYGGFDS |
| 121 | PTVAYSVLVV AKVEKGKSKK LKSVKELLGI TIMERSSFEK |
| 161 | NPIDFLEAKG YKEVKKDLII KLPKYSLFEL ENGRKRMLAS |
| 201 | AGELQKGNEL ALPSKYVNFL YLASHYEKLK GSPEDNEQKQ |
| 241 | LFVEQHKHYL DEIIEQISEF SKRVILADAN LDKVLSAYNK |
| 281 | HRDKPIREQA ENIIHLFTLT NLGAPAAFKY FDTTIDRKRY |
| 321 | TSTKEVLDAT LIHQSITGLY ETRIDLSQLG GDGGSGGSGG |
| 361 | SGGSGGSGGS GGMDKKYSIG LAIGTNSVGW AVITDEYKVP |
| 401 | SKKFKVLGNT DRHSIKKNLI GALLFDSGET AEATRLKRTA |
| 441 | RRRYTRRKNR ICYLQEIFSN EMAKVDDSFF HRLEESFLVE |
| 481 | EDKKHERHPI FGNIVDEVAY HEKYPTIYHL RKKLVDSTDK |
| 521 | ADLRLIYLAL AHMIKFRGHF LIEGDLNPDN SDVDKLFIQL |
| 561 | VQTYNQLFEE NPINASGVDA KAILSARLSK SRRLENLIAQ |
| 601 | LPGEKKNGLF GNLIALSLGL TPNFKSNFDL AEDAKLQLSK |
| 641 | DTYDDDLDNL LAQIGDQYAD LFLAAKNLSD AILLSDILRV |
| 681 | NTEITKAPLS ASMIKRYDEH HQDLTLLKAL VRQQLPEKYK |
| 721 | EIFFDQSKNG YAGYIDGGAS QEEFYKFIKP ILEKMDGTEE |
| 761 | LLVKLNREDL LRKQRTFDNG SIPHQIHLGE LHAILRRQED |
| 801 | FYPFLKDNRE KIEKILTFRI PYYVGPLARG NSRFAWMTRK |
| 841 | SEETITPWNF EEVVDKGASA QSFIERMTNF DKNLPNEKVL |
| 881 | PKHSLLYEYF TVYNELTKVK YVTEGMRKPA FLSGEQKKAI |
| 921 | VDLLFKTNRK VTVKQLKEDY FKKIECFDSV EISGVEDRFN |
| 961 | ASLGTYHDLL KIIKDKDFLD NEENEDILED IVLTLTLFED |
| 1001 | REMIEERLKT YAHLFDDKVM KQLKRRRYTG WGRLSRKLIN |
| 1041 | GIRDKQSGKT ILDFLKSDGF ANRNFMQLIH DDSLTFKEDI |
| 1081 | QKAQVSGQGD SLHEHIANLA GSPAIKKGIL QTVKVVDELV |
| 1121 | KVMGRHKPEN IVIEMARENQ TTQKGQKNSR ERMKRIEEGI |
| 1161 | KELGSQILKE HPVENTQLQN EKLYLYYLQN GRDMYVDQEL |
| 1201 | DINRLSDYDV DAIVPQSFLK DDSIDNKVLT RSDKNRGKSD |
| 1241 | NVPSEEVVKK MKNYWRQLLN AKLITQRKFD NLTKAERGGL |
| 1281 | SELDKAGFIK RQLVETRQIT KHVAQILDSR MNTKYDENDK |
| 1321 | LIREVKVITL KSKLVSDFRK DFQFYKVREI NNYHHAHDAY |
| 1361 | LNAVVGTALI KKYPKLESEF VYGDYKVYDV RKMIAKSEQE |
| 1401 | ITSPKKKRKV* |

Another Cas protein that can be used is the Cas9-CP-1249 circular permutant amino acid sequence (CP15, NLS-Cas9-CP-1249-NLS, cleavage at KLKGS|PEDNE) shown below as SEQ ID NO:48.

| | |
|---|---|
| 1 | MAPKKKRKVS ATEDNEQKQL FVEQHKHYLD EIIEQISEFS |
| 41 | KRVILADANL DKVLSAYNKH RDKPIREQAE NIIHLFTLTN |
| 81 | LGAPAAFKYF DTTIDRKRYT STKEVLDATL IHQSITGLYE |
| 121 | TRIDLSQLGG DGGSGGSGGS GGSGGSGGSG GMDKKYSIGL |
| 161 | DIGTNSVGWA VITDEYKVPS KKFKVLGNTD RHSIKKNLIG |
| 201 | ALLFDSGETA EATRLKRTAR RRYTRRKNRI CYLQEIFSNE |
| 241 | MAKVDDSFFH RLEESFLVEE DKKHERHPIF GNIVDEVAYH |
| 281 | EKYPTIYHLR KKLVDSTDKA DLRLIYLALA HMIKFRGHFL |
| 321 | IEGDLNPDNS DVDKLFIQLV QTYNQLFEEN PINASGVDAK |
| 361 | AILSARLSKS RRLENLIAQL PGEKKNGLFG NLIALSLGLT |
| 401 | PNFKSNFDLA EDAKLQLSKD TYDDDLDNLL AQIGDQYADL |
| 441 | FLAAKNLSDA ILLSDILRVN TEITKAPLSA SMIKRYDEHH |
| 481 | QDLTLLKALV RQQLPEKYKE IFFDQSKNGY AGYIDGGASQ |
| 521 | EEFYKFIKPI LEKMDGTEEL LVKLNREDLL RKQRTFDNGS |
| 561 | IPHQIHLGEL HAILRRQEDF YPFLKDNREK IEKILTFRIP |
| 601 | YYVGPLARGN SRFAWMTRKS EETITPWNFE EVVDKGASAQ |
| 641 | SFIERMTNFD KNLPNEKVLP KHSLLYEYFT VYNELTKVKY |
| 681 | VTEGMRKPAF LSGEQKKAIV DLLFKTNRKV TVKQLKEDYF |
| 721 | KKIECFDSVE ISGVEDRFNA SLGTYHDLLK IIKDKDFLDN |
| 761 | EENEDILEDI VLTLTLFEDR EMIEERLKTY AHLFDDKVMK |
| 801 | QLKRRRYTGW GRLSRKLING IRDKQSGKTI LDFLKSDGFA |
| 841 | NRNFMQLIHD DSLTFKEDIQ KAQVSGQGDS LHEHIANLAG |
| 881 | SPAIKKGILQ TVKVVDELVK VMGRHKPENI VIEMARENQT |
| 921 | TQKGQKNSRE RMKRIEEGIK ELGSQILKEH PVENTQLQNE |
| 961 | KLYLYYLQNG RDMYVDQELD INRLSDYDVD AIVPQSFLKD |
| 1001 | DSIDNKVLTR SDKNRGKSDN VPSEEVVKKM KNYWRQLLNA |
| 1041 | KLITQRKFDN LTKAERGGLS ELDKAGFIKR QLVETRQITK |
| 1081 | HVAQILDSRM NTKYDENDKL IREVKVITLK SKLVSDFRKD |
| 1121 | FQFYKVREIN NYHHAHDAYL NAVVGTALIK KYPKLESEFV |
| 1161 | YGDYKVYDVR KMIAKSEQEI GKATAKYFFY SNIMNFFKTE |
| 1201 | ITLANGEIRK RPLIETNGET GEIVWDKGRD FATVRKVLSM |
| 1241 | PQVNIVKKTE VQTGGFSKES ILPKRNSDKL IARKKDWDPK |
| 1281 | KYGGFDSPTV AYSVLVVAKV EKGKSKKLKS VKELLGITIM |
| 1321 | ERSSFEKNPI DFLEAKGYKE VKKDLIIKLP KYSLFELENG |
| 1361 | RKRMLASAGE LQKGNELALP SKYVNFLYLA SHYEKLKGSP |
| 1401 | ETSPKKKRKV |

Another Cas protein that can be used is the Cas9-CP-1282 circular permutant amino acid sequence (CP16, NLS-Cas9-CP-1282-NLS, cleavage at SKRVI|LADAN), shown below as SEQ ID NO:49.

| | |
|---|---|
| 1 | MAPKKKRKVS AIADANLDKV LSAYNKHRDK PIREQAENII |
| 41 | HLFTLTNLGA PAAFKYFDTT IDRKRYTSTK EVLDATLIHQ |
| 81 | SITGLYETRI DLSQLGGDGG SGGSGGSGGS GGSGGSGGMD |
| 121 | KKYSIGLDIG TNSVGWAVIT DEYKVPSKKF KVLGNTDRHS |
| 161 | IKKNLIGALL FDSGETAEAT RLKRTARRRY TRRKNRICYL |
| 201 | QEIFSNEMAK VDDSFFHRLE ESFLVEEDKK HERHPIFGNI |
| 241 | VDEVAYHEKY PTIYHLRKKL VDSTDKADLR LIYLALAHMI |
| 281 | KFRGHFLIEG DLNPDNSDVD KLFIQLVQTY NQLFEENPIN |
| 321 | ASGVDAKAIL SARLSKSRRL ENLIAQLPGE KKNGLFGNLI |
| 361 | ALSLGLTPNF KSNFDLAEDA KLQLSKDTYD DDLDNLLAQI |
| 401 | GDQYADLFLA AKNLSDAILL SDILRVNTEI TKAPLSASMI |
| 441 | KRYDEHHQDL TLLKALVRQQ LPEKYKEIFF DQSKNGYAGY |
| 481 | IDGGASQEEF YKFIKPILEK MDGTEELLVK LNREDLLRKQ |
| 521 | RTFDNGSIPH QIHLGELHAI LRRQEDFYPF LKDNREKIEK |
| 561 | ILTFRIPYYV GPLARGNSRF AWMTRKSEET ITPWNFEEVV |
| 601 | DKGASAQSFI ERMTNFDKNL PNEKVLPKHS LLYEYFTVYN |
| 641 | ELTKVKYVTE GMRKPAFLSG EQKKAIVDLL FKTNRKVTVK |
| 681 | QLKEDYFKKI ECFDSVEISG VEDRFNASLG TYHDLLKIIK |
| 721 | DKDFLDNEEN EDILEDIVLT LTLFEDREMI EERLKTYAHL |
| 761 | FDDKVMKQLK RRRYTGWGRL SRKLINGIRD KQSGKTILDF |
| 801 | LKSDGFANRN FMQLIHDDSL TFKEDIQKAQ VSGQGDSLHE |
| 841 | HIANLAGSPA IKKGILQTVK VVDELVKVMG RHKPENIVIE |
| 881 | MARENQTTQK GQKNSRERMK RIEEGIKELG SQILKEHPVE |
| 921 | NTQLQNEKLY LYYLQNGRDM YVDQELDINR LSDYDVDAIV |
| 961 | PQSFLKDDSI DNKVLTRSDK NRGKSDNVPS EEVVKKMKNY |
| 1001 | WRQLLNAKLI TQRKFDNLTK AERGGLSELD KAGFIKRQLV |
| 1041 | ETRQITKHVA QILDSRMNTK YDENDKLIRE VKVITLKSKL |
| 1081 | VSDFRKDFQF YKVREINNYH HAHDAYLNAV VGTALIKKYP |
| 1121 | KLESEFVYGD YKVYDVRKMI AKSEQEIGKA TAKYFFYSNI |
| 1161 | MNFFKTEITL ANGEIRKRPL IETNGETGEI VWDKGRDFAT |
| 1201 | VRKVLSMPQV NIVKKTEVQT GGFSKESILP KRNSDKLIAR |
| 1241 | KKDWDPKKYG GFDSPTVAYS VLVVAKVEKG KSKKLKSVKE |
| 1281 | LLGITIMERS SFEKNPIDFL EAKGYKEVKK DLIIKLPKYS |
| 1321 | LFELENGRKR MLASAGELQK GNELALPSKY VNFLYLASHY |
| 1401 | EKLKGSPEDN EQKQLFVEQH KHYLDEIIEQ ISEFSKRVIL |
| 1441 | ATSPKKKRKV |

Another Cas protein that can be used is the ProCas9 amino acid sequence (pCF712 ProCas9-Flavi vector; NLS-Flavivirus protease-sensitive caged ProCas9-NLS) shown below as SEQ ID NO:50.

| | |
|---|---|
| 1 | MAPKKKRKVS ANPINASGVD AKAILSARLS KSRRLENLIA |
| 41 | QLPGEKKNGL FGNLIALSLG LTPNFKSNFD LAEDAKLQLS |
| 81 | KDTYDDDLDN LLAQIGDQYA DLFLAAKNLS DAILLSDILR |
| 121 | VNTEITKAPL SASMIKRYDE HHQDLTLLKA LVRQQLPEKY |
| 161 | KEIFFDQSKN GYAGYIDGGA SQEEFYKFIK PILEKMDGTE |
| 201 | ELLVKLNRED LLRKQRTFDN GSIPHQIHLG ELHAILRRQE |
| 241 | DFYPFLKDNR EKIEKILTFR IPYYVGPLAR GNSRFAWMTR |
| 281 | KSEETITPWN FEEVVDKGAS AQSFIERMTN FDKNLPNEKV |
| 321 | LPKHSLLYEY FTVYNELTKV KYVTEGMRKP AFLSGEQKKA |
| 361 | IVDLLFKTNR KVTVKQLKED YFKKIECFDS VEISGVEDRF |
| 401 | NASLGTYHDL LKIIKDKDFL DNEENEDILE DIVLTLTLFE |
| 441 | DREMIEERLK TYAHLFDDKV MKQLKRRRYT GWGRLSRKLI |
| 481 | NGIRDKQSGK TILDFLKSDG FANRNFMQLI HDDSLTFKED |
| 521 | IQKAQVSGQG DSLHEHIANL AGSPAIKKGI LQTVKVVDEL |
| 561 | VKVMGRHKPE NIVIEMAREN QTTQKGQKNS RERMKRIEEG |
| 601 | IKELGSQILK EHPVENTQLQ NEKLYLYYLQ NGRDMYVDQE |
| 641 | LDINRLSDYD VDHIVPQSFL KDDSIDNKVL TRSDKNRGKS |
| 681 | DNVPSEEVVK KMKNYWRQLL NAKLITQRKF DNLTKAERGG |
| 721 | LSELDKAGFI KRQLVETRQI TKHVAQILDS RMNTKYDEND |
| 761 | KLIREVKVIT LKSKLVSDFR KDFQFYKVRE INNYHHAHDA |
| 801 | YLNAVVGTAL IKKYPKLESE FVYGDYKVYD VRKMIAKSEQ |
| 841 | EIGKATAKYF FYSNIMNFFK TEITLANGEI RKRPLIETNG |
| 881 | ETGEIVWDKG RDFATVRKVL SMPQVNIVKK TEVQTGGFSK |
| 921 | ESILPKRNSD KLIARKKDWD PKKYGGFDSP TVAYSVLVVA |
| 961 | KVEKGKSKKL KSVKELLGIT IMERSSFEKN PIDFLEAKGY |
| 1001 | KEVKKDLIIK LPKYSLFELE NGRKRMLASA GELQKGNELA |
| 1041 | LPSKYVNFLY LASHYEKLKG SPEDNEQKQL FVEQHKHYLD |
| 1081 | EIIEQISEFS KRVILADANL DKVLSAYNKH RDKPIREQAE |
| 1121 | NIIHLFTLTN LGAPAAFKYF DTTIDRKRYT STKEVLDATL |
| 1161 | IHQSITGLYE TRIDLSQLGG DKQKKRGGKD KKYSIGLDIG |
| 1201 | TNSVGWAVIT DEYKVPSKKF KVLGNTDRHS IKKNLIGALL |
| 1241 | FDSGETAEAT RLKRTARRRY TRRKNRICYL QEIFSNEMAK |
| 1281 | VDDSFFHRLE ESFLVEEDKK HERHPIFGNI VDEVAYHEKY |
| 1321 | PTIYHLRKKL VDSTDKADLR LIYLALAHMI KFRGHFLIEG |
| 1361 | DLNPDNSDVD KLFIQLVQTY NQLFEETSPK KKRKV* |

In some cases, the protein is or is encoded by any one of SEQ ID NO: 38-50. In some embodiments, the protein or nucleic acid has about 70%, about 75%, about 80%, about 85%, about 90%, about 95%, about 96%, about 97%, about 98%, about 99%, or more sequence identity to SEQ ID NO: 38-50.

### Guide RNA and Cas Protein/Nuclease Delivery

The guide RNAs and/or proteins can be locally administered or systemically delivered. There are different ways to deliver guide RNAs and Cas proteins. The first approach is to use a vector-based CRISPR-Cas9 system encoding the Cas protein and guide RNA (e.g., sgRNA) from the same vector, thus avoiding multiple transfections or transductions of different components. The second is to deliver the mixture of the Cas9 protein mRNA and the sgRNA, and the third strategy is to deliver the mixture of the Cas9 protein and the sgRNA.

In some cases, the guide RNAs can be delivered to cells or administered to subjects in the form of an expression cassette or vector that can express one or more of the guide RNAs. Cas proteins can also be delivered to cells or administered to the subjects in the form of an expression cassette or vector that can express one or more Cas proteins. The Cas nucleases (e.g. as proteins) can also be combined with their respective gRNAs and delivered as RNA-protein complexes (RNPs). Hence, the RNPs can be pre-assembled outside of the cell and introduced into the cell.

The guide RNAs and/or the Cas proteins/nucleases can include a targeting agent that can restricts the activity of the guide RNAs/nuclease complex to specific targeted cell types (e.g., to specific cancer cell types). The targeting agent can be a protease that is expressed and/or is functional only in the targeted cell type, where the protease activates the Cas protein to have nuclease activity. The targeting agent can be a guide RNA that recognizes only cellular sequences that are unique to the targeted cells. The targeting agent can also be a sequence that localizes a protein within a particular cell type. The targeting agent can, for example, be an antibody or other binding agent that specifically binds to specific cancer cell types and that facilitates delivery of the guide RNAs and the Cas protein (or vector(s) encoding the guide RNAs and the Cas protein/nuclease) to specific targeted cell types.

When the targeting agent is a target cell protease that is functional only in the targeted cell type, the guide RNAs and the Cas protein can be systemically administered. However, in some cases, local delivery may facilitate more rapid uptake and may help avoid non-targeted cellular injury. The target cell protease activates the Cas protein only in the targeted cells (e.g., the targeted cancer cells). The Cas protein can have a modified structure such as the Cas9 circular permutants or ProCas9 enzymes described in the Examples (see also Oakes, Fellmann, et al., Cell 176: 254-267 (2019), which is incorporated by reference herein in its entirety). Such Cas9 circular permutants or ProCas9 enzymes are only activated when cleaved by particular proteases, for example, one or more proteases that are unique to specific cancer cell types. The Cas9 circular permutants or ProCas9 enzymes are therefore selectively activated in presence of a matching cell type specific protease such as a cancer cell specific protease.

Examples of proteases that can activate Cas9 circular permutants include serine proteases, matrix metalloproteinases, aspartic proteases, cysteine proteases, asparaginyl proteases, viral proteases, bacterial proteases, and proteases expressed in a tissue-specific or cell-specific manner. Examples of proteases that can be used also include those listed, for example, in Table 4.

When the targeting agent is a guide RNA that recognizes only cellular sequences that are unique to the targeted cells, the guide RNAs and Cas protein can be systemically delivered. However, in some cases, local delivery may facilitate more rapid uptake and may help avoid non-targeted cellular injury. For example, the guide RNAs can recognize target endogenous cellular sequences that are specific and/or more common in cancer cells compared to the non-cancer cells. Such cancer-cell specific sequences can include specific (somatic) repeat expansions, loci showing cancer-specific copy number amplifications, and/or other repeat sequences that only occur in cancer cells (e.g. due to viral integrations, chromosomal fusion, chromosomal breakpoints, specific somatic mutations, hypermutations following primary treatment, etc.). In such cases, the guide RNAs will only activate the Cas protein in the cell types that have the target endogenous cellular sequences.

Targeting agents that localize a protein (or other molecule) within a cell can, for example, be nuclear localization signal (NLS). Such a nuclear localization sequence has an amino acid sequence that ‘tags’ a protein for import into the cell nucleus by nuclear transport. Typically, this signal consists of one or more short sequences of positively charged lysines or arginines exposed on the protein surface. The nuclear localization sequences can be classified as either monopartite or bipartite. The major structural differences between the two is that the two basic amino acid clusters in bipartite NLSs are separated by a relatively short spacer sequence (hence bipartite—2 parts), while monopartite NLSs are not. The first nuclear localization sequence to be discovered was the sequence PKKKRKV (SEQ ID NO:81) in the SV40 Large T-antigen (a monopartite NLS) (Kalderon et al. Cell. 39: 499-509 (1984)). The NLS of nucleoplasmin, KR[PAATKKAGQA]KKKK (SEQ ID NO:82), is a prototypical bipartite signal: two clusters of basic amino acids, separated by a spacer of about 10 amino acids. Both are recognized by importin α. Importin α contains a bipartite NLS itself, which is specifically recognized by importin β. The importin β may be the actual import mediator.

A comparison of the nuclear localization efficiencies of eGFP fused NLSs of SV40 Large T-Antigen, nucleoplasmin (AVKRPAATKKAGQAKKKKLD, SEQ ID NO:83), EGL-13 (MSRRRKANPTKLSENAKKLAKEVEN, SEQ ID NO:84), c-Myc (PAAKRVKLD, SEQ ID NO:85) and TUS-protein (KLKIKRPVK SEQ ID NO:86) indicated that the c-Myc NLS has higher nuclear localization efficiency compared to that of SV40 NLS (Ray et al., Bioconjug. Chem. 26 (6): 1004-7 (2015)).

When a targeting agent is used that specifically binds to specific cancer cell types. The targeting agent can facilitate delivery of the guide RNAs and the Cas protein (or vector(s) encoding the guide RNAs and the Cas protein) to specific targeted cell types, the combination of the binding agent, the guide RNA(s), and the Cas protein/nuclease (or one or more vectors encoding the guide RNA(s) and the Cas protein/nuclease) can be administered systemically. However, in some cases, local delivery may facilitate more rapid uptake and may help avoid non-targeted cellular injury. The binding agent, the guide RNAs, and the Cas protein/nuclease (or vector(s) encoding the guide RNAs and the Cas protein/nuclease) can be incorporated within a carrier that displays the binding agent. Such a carrier can protect the guide RNAs and the nuclease (or vector(s) encoding the guide RNAs and the Cas protein/nuclease) from degradation and can also protect non-targeted tissues from off-target genomic shredding.

Targeted delivery of the Cas-sgRNA complex to specific cancer cells can include targeted Cas-sgRNA ribonucleoprotein (RNP) delivery using targeting or binding agents that are coupled to the Cas protein or sgRNA; targeted delivery of expression vector(s) encoding the Cas protein/nuclease and/or the gRNA, or a combination thereof. The binding (or targeting) agent can be selective viral vectors, viral particles, or virus like particles (VLPs); or potentially delivery vehicles that are targeted specifically to cancer cells; or nanoparticles that are targeted to cancer cells; or lipid carriers that are targeted to cancer cells. Such nanoparticles, or lipid carriers (e.g., liposomes) can include a binding agent that binds to the targeted cells.

The binding agent can specifically recognize and specifically bind to a cancer marker. A “cancer marker” is a molecule that is differentially expressed or processed in cancer, for example, on a cancer cell or in the cancer milieu. Exemplary cancer markers are cell surface proteins such as cancer cell adhesion molecules, cancer cell receptors, intracellular receptors, hormones, and molecules such as proteases that are secreted by cells into the cancer milieu. Examples include programmed cell death 1 (PD-1; also called CD279), C type Lectin Like molecule 1 (CLL-1), interleukin-1 receptor accessory protein (IL1-RAP, aka IL-1R3). Markers for specific cancers can include CD45 for acute myeloid leukemia, CD34+CD38− for acute myeloid leukemia cancer stem cells, MUC1 expression on colon and colorectal cancers, bombesin receptors in lung cancer, S100A10 protein as a renal cancer marker, and prostate specific membrane antigen (PSMA) on prostate cancer.

The guide RNAs and Cas proteins/nucleases can be recombinantly expressed in the cells. The guide RNAs and Cas protein/nucleases can be introduced in form of a nucleic acid molecules encoding the guide RNAs and/or Cas protein/nucleases. The nucleic acid molecules encoding the guide RNAs and/or Cas protein proteins can be provided in expression cassettes or expression vectors.

The expression cassettes can be within vectors. Vectors can, for example, be expression vectors such as viruses or other vectors that is readily taken up by the cells. Examples of vectors that can be used include, for example, adeno-associated virus (AAV) gene transfer vectors, lentiviral vectors, retroviral vectors, herpes virus vectors, e.g., cytomegalovirus vectors, herpes simplex virus vectors, varicella zoster virus vectors, adenovirus vectors, e.g., helper-dependent adenovirus vectors, adenovirus-AAV hybrids, rabies virus vectors, vesicular stomatitis virus (VSV) vectors, coronavirus vectors, poxvirus vectors and the like. Non-viral vectors may be employed to deliver the expression vectors, e.g., liposomes, nanoparticles, microparticles, lipoplexes, polyplexes, nanotubes, and the like. In one embodiment, two or more expression vectors are administered, for instance, each encoding a distinct guide RNA, a distinct Cas protein, or a combination thereof.

The expression cassettes or expression vectors include promoter sequences that are operably linked to the nucleic acid segment encoding the guide RNAs, Cas proteins, or combinations thereof. The promoter can be heterologous to the nucleic acid segment that includes a guide RNA, a Cas protein, or a combination thereof.

As used herein, the term “heterologous” when used in reference to an expression cassette, expression vector, regulatory sequence, promoter, or nucleic acid refers to an expression cassette, expression vector, regulatory sequence, or nucleic acid that has been manipulated in some way. For example, a heterologous promoter can be a promoter that is not naturally linked to a nucleic acid segment of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.).

Heterologous nucleic acids may comprise sequences that comprise cDNA forms; the cDNA sequences may be expressed in either a sense (to produce mRNA) or anti-sense orientation (to produce an anti-sense RNA transcript that is complementary to the mRNA transcript). Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Methods for ensuring expression of a functional guide RNA, Cas protein, or combinations thereof can involve expression from a transgene, expression cassette, or expression vector. For example, the nucleic acid segments encoding the selected guide RNAs, or combinations thereof can be present in a vector, such as for example a plasmid, cosmid, virus, bacteriophage or another vector available for genetic engineering. The coding sequences inserted in the vector can be synthesized by standard methods or isolated from natural sources. The coding sequences may further be ligated to transcriptional regulatory elements, termination sequences, and/or to other amino acid encoding sequences. Such regulatory sequences can provide initiation of transcription, internal ribosomal entry sites (IRES) (Owens, Proc. Natl. Acad. Sci. USA 98: 1471-1476 (2001)) and optionally regulatory elements ensuring termination of transcription and stabilization of the transcript.

Non-limiting examples for regulatory elements ensuring the initiation of transcription comprise a translation initiation codon, transcriptional enhancers such as e.g. the SV40-enhancer, insulators and/or promoters. The promoter can be a constitutive promoter, and inducible promoter, or a tissue-specific promoter. Examples of promoters that can be used include the cytomegalovirus (CMV) promoter, SV40-promoter, RSV-promoter (Rous sarcoma virus), the lacZ promoter, chicken beta-actin promoter, CAG-promoter (a combination of chicken beta-actin promoter and cytomegalovirus immediate-early enhancer), the gai10 promoter, human elongation factor 1α-promoter, AOX1 promoter, GAL1 promoter CaM-kinase promoter, the lac, trp or tac promoter, the lacUV5 promoter, the Autographa californica multiple nuclear polyhedrosis virus (AcMNPV) polyhedral promoter, or a globin intron in mammalian and other animal cells. Non-limiting examples for regulatory elements ensuring transcription termination include the V40-poly-A site, the tk-poly-A site or the SV40, lacZ or AcMNPV polyhedral polyadenylation signals, which are to be included downstream of the nucleic acid sequence of the invention. Additional regulatory elements may include translational enhancers, Kozak sequences and intervening sequences flanked by donor and acceptor sites for RNA splicing. Moreover, elements such as origin of replication, drug resistance gene or regulators (as part of an inducible promoter) may also be included.

One straightforward approach is to use a vector-based system encoding the Cas protein and guide RNA (e.g., sgRNA) from the same vector, thus avoiding multiple transfections of different components. The second is to deliver the mixture of the Cas9 mRNA and the sgRNA, and the third strategy is to deliver the mixture of the Cas9 protein and the sgRNA.

### Methods

Also described herein are methods that include administering to a patient or subject:
a. at least one guide RNA that binds specifically to a repetitive DNA sequence in a human cell;
b. a composition comprising at least one Cas protein and at least one guide RNA that binds specifically to a repetitive DNA sequence in a human cell;
c. at least one expression system comprising at least one expression cassette, each expression cassette comprising a promoter operably linked to a nucleic acid segment encoding a Cas protein, a guide RNA, or a combination thereof,
d. or a combination thereof.

In some embodiments, the patient or subject suffers from or it is suspected that the patient or subject suffers from a disease or disorder. Such a disease or disorder can be a cell proliferative disease including, but not limited to, one or more leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphomas (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma), or a combination thereof.

For example, in some case the disease or disorder is a glioblastoma.

The methods, compositions, and/or kits described herein can reduce the incidence or progression of such diseases by 1% or more, 2% or more, 3% or more, 5% or more, 7% or more, 10% or more, 15% or more, 20% or more, 25% or more, 30% or more, 40% or more, or 50% or more compared to a control. Such a control can be the initial frequency or previous rate of progression of the disease of the subject. The control can also be an average frequency or rate of progression of the disease. For example, when treating cancer, the compositions and/or methods described herein can reduce tumor volume in the treated subject by 1% or more, 2% or more, 3% or more, 5% or more, 7% or more, 10% or more, 15% or more, 20% or more, 25% or more, 30% or more, 40% or more, or 50% or more compared to a control. Such a control can be the initial tumor volume. In some cases, the compositions and/or methods described herein can reduce the incidence or progression of such diseases by at least 2-fold, or at least 3-fold, or at least 5-fold, or at least 10-fold compared to a control.

### Routes of Administration, Formulations, and Dosages

The disclosed methods of treatment can be accomplished via any mode of administration for therapeutic agents. These modes include systemic or local administration such as oral, nasal, parenteral, transdermal, subcutaneous, vaginal, buccal, rectal or topical administration modes.

Guide RNAs, Cas proteins, or a combination thereof can be administered to subjects. Expression systems that include one or more expression cassettes or expression vectors that can express the guide RNAs, the Cas proteins, or a combination thereof can be administered to subjects. The expression cassettes, expression vectors, and cells are administered in a manner that permits them to be incorporated into, graft or migrate to a specific tissue site, or to specific cell types.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, the compositions can also be administered in intravenous (both bolus and infusion), intraperitoneal, subcutaneous or intramuscular form, and all using forms well known to those skilled in the pharmaceutical arts.

For therapy, expression systems that include one or more expression cassettes or expression vectors can be administered locally or systemically. The expression systems are administered in a manner that permits them to be incorporated into, graft, migrate to a specific tissue site, or migrate to specific cell types. Administration can be by injection, catheter, implantable device, or the like. The expression cassettes, expression vectors, and cells can be administered in any physiologically acceptable excipient or carrier that does not adversely affect the subject. For example, the expression cassettes, expression vectors, and cells can be administered intravenously.

Methods of administering the guide RNAs, Cas proteins, expression systems, or combinations thereof to subjects, particularly human subjects, include injection or implantation of the guide RNAs, Cas proteins, expression systems, or combinations thereof into target sites within a delivery device which facilitates their introduction, uptake, incorporation, targeting, or implantation. Such delivery devices include tubes, e.g., catheters, for introducing cells, expression vectors, and fluids into the body of a recipient subject. The tubes can additionally include a needle, e.g., a syringe, through which the cells of the invention can be introduced into the subject at a desired location. Multiple injections may be made using this procedure.

As used herein, the term “solution” includes a carrier or diluent in which the expression cassettes, expression vectors, and cells of the invention remain viable. Carriers and diluents that can be used include saline, aqueous buffer solutions, solvents and/or dispersion media. The use of such carriers and diluents are available in the art. The solution is preferably sterile and fluid to the extent that easy syringability exists.

The administering the guide RNAs, Cas proteins, expression systems, or combinations thereof can also be embedded in a support matrix. Suitable ingredients include targeting agents, matrix proteins, carriers that support or promote the incorporation of the guide RNAs, Cas proteins, expression systems, or combinations thereof. In another embodiment, the composition may include physiologically acceptable matrix scaffolds. Such physiologically acceptable matrix scaffolds can be resorbable and/or biodegradable.

Liquid, particularly injectable, compositions can, for example, be prepared by dissolution, dispersion, etc. For example, the guide RNAs, Cas proteins, expression systems, or combinations thereof can be dissolved in or mixed with a pharmaceutically acceptable solvent such as, for example, water, saline, aqueous dextrose, glycerol, ethanol, and the like, to thereby form an injectable isotonic solution or suspension.

Carriers, liposomes, nanoparticles, proteins such as albumin, chylomicron particles, or serum proteins can be used to stabilize the guide RNAs, Cas proteins, expression systems, or combinations thereof. Such carriers can also include or display a targeting agent to facilitate delivery to a specific cell type.

The disclosed guide RNAs, Cas proteins, expression systems, or combinations thereof can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the pathway inhibitor and/or modulator of glucose metabolism, as described in U.S. Pat. No. 5,262,564 which is hereby incorporated by reference in its entirety.

Disclosed pharmaceutical compositions can also be delivered by the use of monoclonal antibodies as individual carriers to which the guide RNAs, Cas proteins, expression systems, or combinations thereof are coupled. For example, the monoclonal antibodies can be specific for a selected cell marker, such as a cell surface protein that is unique to a selected target cell. The guide RNAs, Cas proteins, expression systems, or combinations thereof can also be coupled with soluble polymers as targetable drug carriers. Such polymers can include polyvinylpyrrolidone, pyran copolymer, poly(hydroxypropyl)methacrylamide-phenol, poly(hydroxyethyl)-aspanamide phenol, or poly(ethyleneoxide)-polylysine substituted with palmitoyl residues. Furthermore, the guide RNAs, Cas proteins, expression systems, or combinations thereof can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Pharmaceutical compositions can be prepared according to mixing, granulating or coating methods, and the compositions can contain from about 0.1% to about 99%, from about 5% to about 90%, or from about 1% to about 20% of guide RNAs, Cas proteins, expression systems, or combinations thereof by weight or volume.

The dosage regimen is selected in accordance with a variety of factors including type, species, age, weight, sex and medical condition of the subject; the severity of the condition to be treated; the route of administration; the renal or hepatic function of the subject; and the particular guide RNAs, Cas proteins, expression systems, or combinations thereof employed. A physician or veterinarian of ordinary skill in the art can readily determine and prescribe the effective amount of the guide RNAs, Cas proteins, expression systems, or combinations thereof required to prevent, counter or arrest the progress of the disease or disorder.

The guide RNAs, Cas proteins, expression systems, or combination thereof may be administered in a composition as a single dose, in multiple doses, in a continuous or intermittent manner, depending, for example, upon the recipient's physiological condition, whether the purpose of the administration is for more sustained therapeutic purposes, and other factors known to skilled practitioners. The administration of the compositions of the invention may be provided as a single dose, or essentially continuous over a preselected period of time, or it may be in a series of spaced doses. Both local and systemic administration is contemplated.

In some cases, effective dosage amounts of the guide RNAs, Cas proteins, expression systems, or combinations thereof when used for the indicated effects, range from about 0.5 mg to about 5000 mg as needed to treat the disease or disorder. Compositions for in vivo or in vitro use can contain about 0.5, 5, 20, 50, 75, 100, 150, 250, 500, 750, 1000, 1250, 2500, 3500, or 5000 mg of the guide RNAs, Cas proteins, expression systems, or combinations thereof, or, in a range of from one amount to another amount in the list of doses.

Hence, the disclosure provides a pharmaceutical composition that include any of the guide RNAs, Cas proteins, expression systems, or combinations thereof described herein.

The compositions can also contain other ingredients such as chemotherapeutic agents, anti-viral agents, antibacterial agents, antimicrobial agents and/or preservatives. Examples of additional therapeutic agents that may be used include, but are not limited to: anti-PD-L1 antibodies, alkylating agents, such as nitrogen mustards, alkyl sulfonates, nitrosoureas, ethylenimines, and triazenes; antimetabolites, such as folate antagonists, purine analogues, and pyrimidine analogues; antibiotics, such as anthracyclines, bleomycins, mitomycin, dactinomycin, and plicamycin; enzymes, such as L-asparaginase; farnesyl-protein transferase inhibitors; hormonal agents, such as glucocorticoids, estrogens/antiestrogens, androgens/antiandrogens, progestins, and luteinizing hormone-releasing hormone anatagonists, octreotide acetate; microtubule-disruptor agents, such as ecteinascidins or their analogs and derivatives; microtubule-stabilizing agents such as paclitaxel (Taxol®), nab-paclitaxel, docetaxel (Taxotere®), and epothilones A-F or their analogs or derivatives; plant-derived products, such as vinca alkaloids, epipodophyllotoxins, taxanes; and topoisomerase inhibitors; prenyl-protein transferase inhibitors; and miscellaneous agents such as, hydroxyurea, procarbazine, mitotane, hexamethylmelamine, platinum coordination complexes such as cisplatin and carboplatin; and other agents used as anti-cancer and cytotoxic agents such as biological response modifiers, growth factors; immune modulators, and monoclonal antibodies. The compositions can also be used in conjunction with radiation therapy.

### Kits

Also described herein is a kit that includes a packaged composition for controlling, preventing or treating a cell proliferative disease or cell proliferation disease.

In one embodiment, the kit or container holds at least one guide RNA described herein and instructions for using the guide RNA. Such a kit can also include at least one Cas protein. The instructions can include a description for using at least one Cas protein with at least one guide RNA. The guide RNA and the Cas protein can be packaged either separately in different containers, or together in a single container.

In some cases, the kit can include an expression system that includes at least one expression cassette having a promoter operably linked to a nucleic acid segment that includes a guide RNA, a Cas protein, or a combination thereof. The promoter can be heterologous to the nucleic acid segment that includes a guide RNA, a Cas protein, or a combination thereof. The expression system can be encapsulated in a liposome, nanoparticle, or other carrier. Similarly, the kit can include a liposome, nanoparticle, or carrier with at least one guide RNA, at least one Cas protein, or a combination thereof.

The kit can also hold instructions for administering the at least one guide RNA, at least one a Cas protein, or a combination thereof. The kit can also include instructions for administering an expression system that includes at least one expression cassette having a promoter operably linked to a nucleic acid segment that includes a guide RNA, a Cas protein, or a combination thereof.

The kits of the invention can also include containers with tools useful for administering the compositions and maintaining a ketogenic diet as described herein. Such tools include syringes, swabs, catheters, antiseptic solutions, package opening devices, forks, spoons, straws, and the like.

The compositions, kits, and/or methods described herein are useful for treatment of cell proliferative diseases such as cancer or cell-proliferative disorder.

For example, the compositions, kits, and/or methods described herein can reduce the incidence or progression of such diseases by 1% or more, 2% or more, 3% or more, 5% or more, 7% or more, 10% or more, 15% or more, 20% or more, 25% or more, 30% or more, 40% or more, or 50% or more compared to a control. Such a control can be the initial frequency or previous rate of progression of the disease of the subject. The control can also be an average frequency or rate of progression of the disease. For example, when treating cancer, the compositions and/or methods described herein can reduce tumor volume in the treated subject by 1% or more, 2% or more, 3% or more, 5% or more, 7% or more, 10% or more, 15% or more, 20% or more, 25% or more, 30% or more, 40% or more, or 50% or more compared to a control. Such a control can be the initial tumor volume. In some cases, the compositions and/or methods described herein can reduce the incidence or progression of such diseases by at least 2-fold, or at least 3-fold, or at least 5-fold, or at least 10-fold compared to a control.

The following Examples illustrate experiments and experimental results performed during development of the invention.

### Example 1: Materials and Methods

This Example illustrates some of the materials and methods that were used in the development of the invention.

### Bacterial Strains and Media

For in-vivo E. coli screening, fluorescence measurements, and cell proliferation assays, MG1655 was used with a chromosomally integrated and constitutively expressed green fluorescent protein (GFP) and red fluorescent protein (RFP) (Oakes et al., 2014; Qi et al., 2013). EZ-rich defined growth medium (EZ-RDM, Teknoka) was used for all liquid culture assays and plates were made using 2×YT. Plasmids used were based on a 2-plasmid system as reported previously (Oakes et al., 2014, 2016; Qi et al., 2013) containing Cas9 and variants on a selectable chloramphenicol-resistant (Cm^{R}) marker and plasmids with sgRNAs and proteases with Amp^{R} markers. The antibiotics were used to verify transformation and to maintain plasmid stocks. No blinding or randomization was done for any of the experiments reported.

### Mammalian Cell Culture

All mammalian cell cultures were maintained in a 37° C. incubator, at 5% carbon dioxide. HEK293T (293FT; Thermo Fisher Scientific, #R70007) human kidney cells and derivatives thereof were grown in Dulbecco's Modified Eagle Medium (DMEM; Corning Cellgro, #10-013-CV) supplemented with 10% fetal bovine serum (FBS; Seradigm, #1500-500), and 100 Units/ml penicillin and 100 μg/ml streptomycin (100-Pen-Strep; GIBCO #15140-122). HepG2 human liver cells (ATCC, #HB-8065) and derivatives thereof were cultured in Eagle's Minimum Essential Medium (EMEM; ATCC, #30-2003) supplemented with 10% FBS and 100-Pen-Strep. A549 human lung cells (ATCC, #CCL-185) and derivatives thereof were grown in Ham's F-12K Nutrient Mixture, Kaighn's Modification (F-12K; Corning Cellgro, #10-025-CV) supplemented with 10% FBS and 100-Pen-Strep. HAP1 cells (kind gift from Jan Carette, Stanford) and derivatives thereof were grown in Iscove's Modified Dulbecco's Medium (IMDM; GIBCO #12440-053 or HyClone #SH30228.01) supplemented with 10% FBS and 100-Pen-Strep. HAP1 cells had been derived from the near-haploid chronic myeloid leukemia cell line KBM7 (Carette et al., 2011). Karyotyping analysis demonstrated that most cells (27 of 39) were fully haploid, while a smaller population (9 of 39) was haploid for all chromosomes except chromosome 8, like the parental KBM7 cells. Less than 10% (3 of 39) were diploid for all chromosomes except for chromosome 8, which was tetraploid.

A549 cells were authenticated using short tandem repeat DNA profiling (STR profiling; UC Berkeley Cell Culture/DNA Sequencing facility). STR profiling was carried out by PCR amplification of nine STR loci plus amelogenin (GenePrint 10 System; Promega #B9510), fragment analysis (3730XL DNA Analyzer; Applied Biosystems), comprehensive data analysis (GeneMapper software; Applied Biosystems), and final verification using supplier databases including American Type Culture Collection (ATCC) and Deutsche Sammlung von Mikroorganismen und Zellkulturen (DSMZ).

HEK293T, HEK-RT1, HEK-RT6, HepG2, A549, and HAP1 cells were tested for absence of Mycoplasma contamination (UC Berkeley Cell Culture facility) by fluorescence microscopy of methanol fixed and Hoechst 33258 (Polysciences #09460) stained samples.

U-251 human glioblastoma cells (Sigma-Aldrich, #09063001;

RRID:CVCL_0021), LN-229 human glioblastoma cells (ATCC, #CRL-2611;
RRID:CVCL_0393), T98G human glioblastoma cells (ATCC, #CRL-1690;
RRID:CVCL_0556), LN-18 human glioblastoma cells (ATCC, #CRL-2610;
RRID:CVCL_0392), and derivatives thereof were cultured in Dulbecco's Modified Eagle Medium/Nutrient Mixture F-12 (DMEM/F-12; Gibco, #11320-033 or Corning Cellgro, #10-090-CV) supplemented with 10% FBS and 100-Pen-Strep. U-251, LN-229, T98G, LN-18, and HEK293T cells were authenticated using short tandem repeat DNA profiling (STR profiling; UC Berkeley Cell Culture/DNA Sequencing facility). STR profiling was carried out by PCR amplification of nine STR loci plus amelogenin (GenePrint 10 System; Promega, #B9510), fragment analysis (3730XL DNA Analyzer; Applied Biosystems), comprehensive data analysis (GeneMapper software; Applied Biosystems), and final verification using supplier databases including American Type Culture Collection (ATCC) and Deutsche Sammlung von Mikroorganismen und Zellkulturen (DSMZ). U-251, LN-229, T98G, LN-18, and HEK293T cells were tested for absence of Mycoplasma contamination (UC Berkeley Cell Culture facility) by fluorescence microscopy of methanol fixed and Hoechst 33258 (Polysciences, #09460) stained samples.

### Plasmid and Viral Vectors

The plasmid vector pCF153, expressing the Gag-Pol polyprotein from Friend murine leukemia virus FB29 (GenBank: Z11128.1), was derived from the pGagPol insert and pVSV-G backbone (a kind gift from Philippe Mangeot, Inserm) (Mangeot et al., 2019) to optimize vector size and expression efficiency. The plasmid vector pCF160, expressing the vesicular stomatitis virus glycoprotein (VSV-G), was derived from pVSV-G to optimize the Kozak sequence. The lentiviral vector pCF226, expressing Streptococcus pyogenes Cas9 and a puromycin selection marker, was described previously (Oakes et al., 2019). The lentiviral vector pCF821, encoding a U6-sgRNA cassette and an EF1a driven mNeonGreen marker, was derived from the pCF525 backbone (Watters et al., 2018) and the pCF221-based U6-sgRNA-EF1a-mCherry insert (Oakes et al., 2019). The mCherry fluorescence marker was replaced with a human codon optimized version of mNeonGreen (gBlock, Integrated DNA Technologies). Analogously, the lentiviral vector pCF820, encoding a U6-sgRNA-EF1a-mCherry2 cassette, was derived from pCF821 by replacing the mNeonGreen marker with a human codon optimized version of mCherry2 (gBlock, Integrated DNA Technologies). Of note, both the pCF820 (mCherry2) and pCF821 (mNeonGreen) sgRNA vectors yield higher viral titers than the otherwise comparable sgRNA vector pCF221 (mCherry). The all-in-one lentiviral vector pCF826, featuring a U6-sgRNA and EFS-Cas9-mCherry2 cassette, was derived from pCF820 with an EFS-Cas9 insert from pCF226 (Oakes et al., 2019). The all-in-one retroviral vector pCF841, encoding a U6-sgRNA and EFS-Cas9-mNeonGreen cassette, was derived from pCF826 by replacing mCherry2 with mNeonGreen from pCF821 and by replacing the lentiviral LTR elements (5′ LTR, packaging signal, RRE, cPPT/CTS, self-inactivating 3′ LTR; human immunodeficiency virus-derived) with retroviral LTR elements (5′ LTR, packaging signal, truncated gag, self-inactivating 3′ LTR; murine leukemia virus-derived) from the RT3GEPIR vector (Fellmann et al., 2013).

### Transposon Library Construction

To begin, a defective Cas9 (dCas9) coding region flanked by BsaI restriction enzyme sites was inserted into a pUC19 based plasmid. A modified transposon with R1 and R2 sites (Jones et al., 2016), containing a chloramphenicol antibiotic resistance marker, p15A origin of replication, TetR and TetR/A promoter, was built using custom oligos and standard molecular biology techniques. The modified transposon was then cleaved from a plasmid using HindIII and gel purified. This linear transposon product was used in overnight in vitro reactions (0.5 molar ratio transposon to 100 ng dCas9-Puc19 plasmid) with 1 mL of MuA Transposase (F-750, Thermo Fisher) in 10 replicates. The transposed DNA was purified and recovered. Plasmids were electroporated into custom made electrocompetent MG1655 E. coli (Oakes et al., 2014) using a BTX Harvard apparatus ECM630 High Throughput Electroporation System and titered on carbenicillin (Carb) and chloramphenicol (CM) to ensure greater than 100× coverage of the library size (13,614). These cells were then outgrown for 12 hours and selected for via Carb and CM markers to ensure growth of transposed members. After isolating transposed plasmids via miniprep (QIAGEN), the original Puc19 backbone was removed via BsaI cleavage and dCas9 proteins transposed with a new plasmid backbone were selected via a 0.7% TAE agarose gel. The linear fragments were then ligated overnight with annealed and phosphorylated oligos coding for GGS linkers encoding 5, 10, 15 and 20 amino acids using a BsaI Golden Gate reaction. Completed libraries were purified, electroporated into the E. coli Mg1655 RFP and GFP screening strain containing an RFP-repressing sgRNA, and the electroporated cells were titered on carbenicillin (Carb) and chloramphenicol (CM) to ensure >5× coverage of the library size (8,216).

### Screening for Cas9 Circular Permutants (Cas9-CPs)

Screens were performed in a similar manner to previous reports (Oakes et al., 2014, 2016). Briefly, biological duplicates of Cas9-CP libraries with an RFP guide RNA were transformed (at greater than 5× library size) into E. coli MG1655 with genetically integrated and constitutively expressed GFP and RFP. Cells were grown overnight in EZ-RDM+Carb, CM, and 200 nM Anhydrotetracycline (aTc) inducer. E. coli were then sorted based on gates for RFP repression but not GFP repression, the RFP-repressed, GFP-expressing cells were collected, and the cells were resorted immediately to further enrich for functional Cas9-CPs. Double sorted libraries were then grown out and DNA was collected for sequencing. This DNA was also retransformed onto plates and individual clones were picked for further analysis.

### Deep Sequencing Library Preparation

This method was modified from previous Tnseq protocols (e.g., Coradetti et al., 2018). Briefly, the transposed plasmids were sheared to about 300 bp using a S220 Focused-ultrasonicator (Covaris) and purified in between each of the following steps using Agencourt AMPure XP beads (Beckman Coulter). Following shearing, fragments were end-repaired and A-tailed according to NEB manufacturers protocols, and then universal adapters were ligated onto the fragments in a 50 ul quick ligase reaction at room temperature. Fragments from each library were then amplified in a 20-cycle reaction with Indexed Illumina primers that annealed upstream of the new CP start codon and in the universal adaptor. PCR products were cleaned again and analyzed for primer dimers via an Agilent Bioanalyzer DNA 1000 chip. Sequencing was performed at the QB3 Vincent J. Coates Genomics Sequencing Laboratory on a HiSeq2500 in a 100 bp run.

### Deep Sequencing Analysis

Demultiplexed reads from the HiSeq2500 were assessed using FastQC to check basic quality metrics. Reads for each sample were then trimmed using a custom python script. The trimmed sequences were mapped to the dCas9 nucleic acid sequence using BWA via a custom python wrapper script to determine the amino acid position in dCas9 corresponding to the starting amino acid position in the dCas9-CP permutant. The resulting alignment files were then processed using a custom python script to calculate the abundance of each dCas9-CP permutant in a given library sample. Fold-changes for each dCas9-CP permutant between pre-library and post-library sorts along with significance values for each enrichment were calculated using the DESeq package in R (Anders and Huber, 2010). Due to ambiguity in transposon sequence, insertion site calls were one greater (sites: n+1) than the variants named in Table 3. As per the DESeq guidelines, count data from technical sequencing replicates were summed to create one unique replicate before running through the DESeq pipeline. All relevant sequencing data and Cas9-CP analysis scripts are available in a website at github.com/SavageLab/cpCas9.

E. coli CRISPRi GFP Repression Assay

Assays were performed using methods like those described by Oakes et al. (2016). To measure the ability of a circular permutant to bind to and repress DNA expression, cells were co-transformed with a Cas9 permutant plasmid with aTc inducible promoter and a single guide RNA plasmid for RFP or GFP that, in the case of the ProCas9 assays, also contained the active or inactive proteases on an IPTG-inducible promoter.

Endpoint Assay: Cells were picked in biological triplicate into 96 well plates containing 500 μL EZ MOPS plus Carb and CM. Plates were grown in 37° C. shakers for twelve hours. Next, cells were diluted 1:1000 in 500 μL EZ MOPS plus Carb, CM, IPTG and aTc. Two hundred nM aTc was used to induce Cas9-CPs or ProCas9s and 50 μM IPTG levels was used to induce the proteases in a 2 mL deep well blocks and shaken at 750 rpm at 37° C. After an eight-twelve-hour induction and growth period, 20 μL of cells were added to 80 μL of water and put into a 96-well microplate reader (Tecan M1000) at 37° C. and read immediately. Each well was measured for optical density at 600 nm and GFP or RFP fluorescence. GFP expression was normalized by dividing it with OD600. In the case of the time course assays, 150 μL of the 1:1000 dilution was used and placed into a black walled clear bottom plate (3631-Corning) and directly into the Tecan M1000 for a 130× 600 s kinetic cycle of reading. For E. coli single cell analysis, cells from the endpoint time course were run on a Sony SH800 to capture 100,000 events per sample.

E. coli Genomic Cleavage Assay

Assays were performed as previously described (Oakes et al., 2016) E. coli containing sgRNA plasmids targeting a genomically integrated GFP were made electrocompetent and transformed with 10 ng of the various Cas9-CP plasmids or controls using electroporation. After recovery in 1 mL SOC media for 1 hour, cells were plated in technical triplicate of tenfold serial dilutions onto 2×YT agar plates with antibiotics selection for both plasmids and aTc induction at 200 nM. Plates were grown at 37° C. overnight and CFU/mL was determined. A reduction in CFUs indicated genomic cleavage and cell death.

E. coli Western Blotting

After CRISPRi repression assays for TEV linker Pro-Cas9s, 40 μL of cell culture was pelleted and resuspended in SDS loading buffer for further analysis. SDS samples were loaded into 4%-20% acrylamide gels (BioRad) for electrophoresis. After transfer to membranes (Trans-Blot Turbo-BioRad), blots were washed three times with 1×TBS+0.01% Tween 20, blocked with 5% milk for 1.5 hour and then a 1:1000 of HRP-conjugated DYKDDDDK (SEQ ID NO:51) Tag (Anti-Flag) antibody (Cell Signaling Technology, #2044) was incubated for twenty-four hours at 4° C. Antibodies were washed away with 3×TBST and detected using Pierce ECL Western Blotting Substrate (Thermo Fisher).

### NIa Protease Cleavage Sites

NIa protease cleavage sites—i.e., the CP linkers—were identified from previous reports (TuMV, 7 aa; Kim et al., 2016), by using the sequence between the P3 and 6KI genes annotated in NCBI (PPV, PVY, CBSV), or from previously identified Potyvirus protease consensus sequences (Seon Han et al., 2013).

### Lentiviral Vectors

A lentiviral vector referred to as pCF204, expressing a U6 driven sgRNA and an EFS driven Cas9-P2A-Puro cassette, was based on the lenti-CRISPR-V2 plasmid (Sanjana et al., 2014), by replacing the sgRNA with an enhanced Streptococcus pyogenes Cas9 sgRNA scaffold (Chen et al., 2013). The pCF704 and pCF711 lentiviral vectors, expressing a U6-sgRNA and an EFS driven ProCas9 variant, were derived from pCF204 by swapping wild-type Cas9 for the respective ProCas9 variant. The pCF712 and pCF713 vectors were derived from pCF704 and pCF711, respectively, be replacing the EF1a-short promoter (EFS) with the full-length EF1a promoter. The lentiviral vector pCF732 was derived from pCF712 by removal of the ProCas9's nuclear localization sequences (NLSs). Vectors not containing a guide RNA, including pCF226 (Cas9-wt) and pCF730 (ProCas9Flavi), were derived from pCF204 and pCF712, respectively, through KpnI/NheI-based removal of the U6-sgRNA cassette and blunt ligation. The guide RNA-only vector pCF221, encoding a U6-sgRNA cassette and an EF1a driven mCherry marker, is loosely based on the pCF204 backbone and guide RNA cassette. Lentiviral vectors expressing viral proteases, including pCF708 expressing an EF1a driven mTagBFP2-tagged dTEV protease, pCF709 expressing an EF1a driven mTagBFP2-tagged ZIKV NS2B-NS3 protease, and pCF710 expressing an EF1a driven mTagBFP2-tagged WNV protease, are all based on the pCF226 backbone. The GFP-tagged protease vectors pCF736 and pCF738 are derived from pCF708 and pCF710, respectively, by swapping mTagBFP2 with GFP. All vectors were generated using custom oligonucleotides (IDT), gBlocks (IDT), standard cloning methods, and Gibson assembly techniques and reagents (NEB).

Design of sgRNAs

Standard sgRNA sequences were either designed manually, using CRISPR Design (crispr.mit.edu), or using GuideScan (Perez et al., 2017). When editing endogenous genes, sgRNAs were often designed to target evolutionarily conserved regions in the 50 proximal third of the gene of interest. The following sequences were used: sgGFP1 (CCTCGaaCTTCACCTCGGCG, SEQ ID NO:52), sgGFP2 (CaaCTACaa GACCCGCGCCG, SEQ ID NO:53), sgGFP9 (CCGGCaaGCTGCCCGTGCCC, SEQ ID NO:54), sgOR2B6-1 (CATTATTCTAGTGTCACGCC, SEQ ID NO:55), sgOR2B6-2 (GGGTATGaaGTTTGGTGTCC, SEQ ID NO:56), sgPCSK9-4 (CCGGTGGTCACT CTGTATGC, SEQ ID NO:57), sgPuro5 (TGTCGAGCCCGACGCGCGTG, SEQ ID NO:58), sgPuro6 (GCTCGGTGACCCGCTCGATG, SEQ ID NO:59), sgRPA1-1 (ACaaaaGTCAGATCCGTACC, SEQ ID NO:60), sgRPA1-2 (TACCTGGAGCaa CTCCCGAG, SEQ ID NO:62). All sgRNAs were designed with a G preceding the 20-nucleotide guide for better expression from U6 promoters.

To enable rapid CRISPR-Cas controlled cell depletion, through a strategy that was termed Cas-induced death by editing or ‘CIDE’, several sgRNAs (sgCIDEs) were designed directed again highly repetitive sequences in the human genome. In brief, using GuideScan (Perez et al., 2017) the most frequently occurring Streptococcus pyogenes Cas9 sgRNA target sites (50-NGG-30 PAM) were identified in the hg38 assembly (Genome Reference Consortium Human Build 38) of the human genome. Sequences were eliminated from this list that contained extended homomeric stretches (greater than four A/T/C/or G). Two sequences (sgCIDE-4, CGCCTGTaaTCCCAGCACTT (SEQ ID NO:63); sgCIDE-5, CCTCGGCCTCCCaaAGTGCT (SEQ ID NO:64) were empirically validated with slightly over 125,000 target loci. Two additional sequences (sgCIDE-1, TGTaaTCCCAGCACTTTGGG (SEQ ID NO:65); sgCIDE-2, TCCCaaAGT GCTGGGATTAC (SEQ ID NO:66) were empirically validated with approximately 300,000 target loci. All four sgCIDEs led to rapid cell depletion when expressed in presence of active Cas9.

All sgRNA sequences provided in Table 2 were cloned into the pCF820, pCF821, and pCF826 vectors using Esp3I restriction sites and enzymes (New England Biolabs). Because the pCF841 vector contains additional Esp3I sites, U6-sgRNA cassettes were PCR amplified from other vectors and inserted into XhoI/EcoRI-HF digested pCF841 using Gibson assembly (New England Biolabs).

### CRISPR-Safe Packaging Cells

To prevent viral packaging cells from dying when transfecting all-in-one Cas9-sgRNA vectors expressing sgCIDEs, HEK293T human embryonic kidney cells (293FT; Thermo Fisher Scientific, #R70007; RRID:CVCL_6911) were transduced with the lentiviral vector pCF525-AcrIIA4 (Watters et al., 2018, 2020) to stably express the anti-CRISPR protein AcrIIA4, a potent inhibitor of Streptococcus pyogenes Cas9 (Rauch et al., 2017). Transduced cells were selected on Hygromycin B (400 μg/ml; Thermo Fisher Scientific, #10687010) and the resulting cell line termed “CRISPR-Safe” packaging cells.

### Lentiviral Transduction

Lentiviral particles were produced in HEK293T cells using polyethylenimine (PEI; Polysciences #23966) based transfection of plasmids. HEK293T cells were split to reach a confluency of 70%-90% at time of transfection. Lentiviral vectors were co-transfected with the lentiviral packaging plasmid psPAX2 (Addgene #12260) and the VSV-G envelope plasmid pMD2.G (Addgene #12259). Transfection reactions were assembled in reduced serum media (Opti-MEM; GIBCO #31985-070). For lentiviral particle production on 10 cm plates, 8 μg lentiviral vector, 4 μg psPAX2 and 2 μg pMD2.G were mixed in 2 mL Opti-MEM, followed by addition of 42 μg PEI. After 20-30 min incubation at room temperature, the transfection reactions were dispersed over the HEK293T cells. Media was changed 12-hour post-transfection, and virus harvested at 36-48-hour post-transfection. Viral supernatants were filtered using 0.45 μm cellulose acetate or polyethersulfone (PES) membrane filters, diluted in cell culture media if appropriate, and added to target cells. Polybrene (5 μg/ml; Sigma-Aldrich) was supplemented to enhance transduction efficiency, if necessary.

Transduced target cell populations (HEK293T, A549, HAP1, HepG2 and derivatives thereof) were usually selected 24-48-hour post-transduction using puromycin (InvivoGen #ant-pr-1; HEK293T, A549 and HepG2: 1.0 μg/ml, HAP1: 0.5 μg/ml) or hygromycin B (Thermo Fisher Scientific #10687010; 200-400 μg/ml).

### Viral Transduction

In general, to enable high viral titers, both lentiviral and retroviral all-in-one particles encoding Cas9-sgRNA (sgCIDE) were produced using the established CRISPR-Safe packaging cell line described herein. Generally, lentiviral particles were produced in HEK293T cells or derivatives thereof using polyethylenimine (PEI; Polysciences #23966) mediated transfection of plasmids, as previously described (Oakes et al., 2019). In brief, lentiviral transfer vectors were co-transfected with the lentiviral helper plasmid psPAX2 (Addgene #12260) and the VSV-G envelope plasmid pMD2.G (Addgene, #12259). Transfection reactions were assembled in reduced serum media (Opti-MEM; Gibco, #31985-070). For lentiviral particle production on 6-well plates, 1 μg lentiviral vector, 0.5 μg psPAX2 and 0.25 μg pMD2.G were mixed in 0.4 ml Opti-MEM, followed by addition of 5.25 μg PEI. After 20-30 min incubation at room temperature, the transfection reactions were dispersed over the HEK293T cells. Media was changed 12-14 h post-transfection, and virus harvested at 42-48 h post-transfection. Viral supernatants were filtered using 0.45 μm polyethersulfone (PES) membrane filters, diluted in cell culture media as appropriate, and added to target cells. Polybrene (5 μg/ml; Sigma-Aldrich) was supplemented to enhance transduction efficiency, if necessary. Similarly, retroviral particles were also produced in HEK293T cells or derivatives thereof using polyethylenimine (PEI; Polysciences #23966) mediated transfection of plasmids. Specifically, retroviral transfer vectors were co-transfected with the retroviral helper plasmids pCF153 (expressing Gag-Pol from FMLV) and pCF160 (expressing the envelope protein VSV-G). Transfection reactions were assembled in reduced serum media (Opti-MEM; Gibco, #31985-070). For retroviral particle production on 6-well plates, 1 μg retroviral transfer vector, 0.5 μg pCF153 and 0.25 μg pCF160 were mixed in 0.4 ml Opti-MEM, followed by addition of 5.25 μg PEI. After 20-30 min incubation at room temperature, the transfection reactions were dispersed over the HEK293T cells. Media was changed 12-14 h post-transfection, and virus harvested at 42-48 h post-transfection. Viral supernatants were filtered using 0.45 μm polyethersulfone (PES) membrane filters, diluted in cell culture media as appropriate, and added to target cells. Polybrene (5 μg/ml; Sigma-Aldrich) was supplemented to enhance transduction efficiency, if necessary.

### Rapid Mammalian Genome Editing Reporter Assay

To establish a rapid and quantitative way to reliably assess genome editing efficiency from various CRISPR-Cas constructs in mammalian cells, a fluorescence-based reporter assay was built. Assays leveraging editing-based disruption of a constitutively expressed fluorescence marker have been built before. However, such assays show a long detection lag time as the genetic disruption of a locus coding for the fluorescent marker would not immediately lead to a reduction in the fluorescence signal, due to the remaining presence of intact transcripts and protein half-life. To quantify this effect, HEK293T cells were stably transduced with a retroviral vector (LMP-Pten.1524) constitutively expressing GFP (Fellmann et al., 2013), and established monoclonal derivatives. The best performing cell line was termed HEK-LMP-10. When editing this reporter line with a vector (pX459, Addgene #48139) expressing wild-type Streptococcus pyogenes Cas9 and guide RNAs targeting the reporter (sgGFP1, sgGFP2), or a non-targeting control (sgNT), the editing detection lag—defined as the time between introduction of an editing reagent and complete loss of fluorescence signal in edited cells—was up to eight days. Hence, this type of assay is inconvenient for rapid quantification of editing efficiency. Conversely, assays relying on frameshift mutations to activate a fluorescence reporter often require specific guide RNA sequences and only get activated with the faction of edits that lead to the required frameshift, thus introducing a quantification bias.

To overcome this limitation, an inducible genome editing reporter cell line was built that had a fluorescence marker that is not expressed in the default state but can be induced following a defined time of potential genome editing. In this scenario, unedited cells rapidly turn positive, while non-edited cells remain fluorophore negative. Specifically, inducible monoclonal HEK293T-based genome editing reporter cells, referred to as “HEK-RT1,” were established in a two-step procedure. In the first step, puromycin resistant monoclonal HEK-RT3-4 reporter cells were generated (Park et al., 2018). In brief, HEK293T human embryonic kidney cells were transduced at low-copy with the amphotropic pseudotyped RT3GEPIR-Ren.713 retroviral vector (Fellmann et al., 2013), comprising an all-in-one Tet-On system enabling doxycycline-controlled GFP expression. After puromycin (2.0 μg/ml) selection of transduced HEK239 Ts, 36 clones were isolated and individually assessed for i) growth characteristics, ii) homogeneous morphology, iii) sharp fluorescence peaks of doxycycline (1 μg/ml) inducible GFP expression, iv) relatively low fluorescence intensity to favor clones with single-copy reporter integration, and v) high transfectability. HEK-RT3-4 cells are derived from the clone that performed best in these tests.

Since HEK-RT3-4 are puromycin resistant, in the second step, monoclonal HEK-RT1 and analogous sister reporter cell lines were derived by transient transfection of HEK-RT3-4 cells with a pair of vectors encoding Cas9 and guide RNAs targeting puromycin (sgPuro5, sgPuro6), followed by identification of monoclonal derivatives that are puromycin sensitive. In total, eight clones were isolated and individually assessed for i) growth characteristics, ii) homogeneous morphology, iii) doxycycline (1 μg/ml) inducible and reversible GFP fluorescence, and v) puromycin and hygromycin B sensitivity. The monoclonal HEK-RT1 and HEK-RT6 cell lines performed best in these tests and were further evaluated in a doxycycline titration experiment, showing that both reporter lines enable doxycycline concentration-dependent induction of the fluorescence marker in as little as 24-48 hours. The HEK-RT1 cell line was chosen as rapid mammalian genome editing reporter system for all further assays.

### Genome Editing Analysis Using the Mammalian HEK-RT1 Reporter Assay

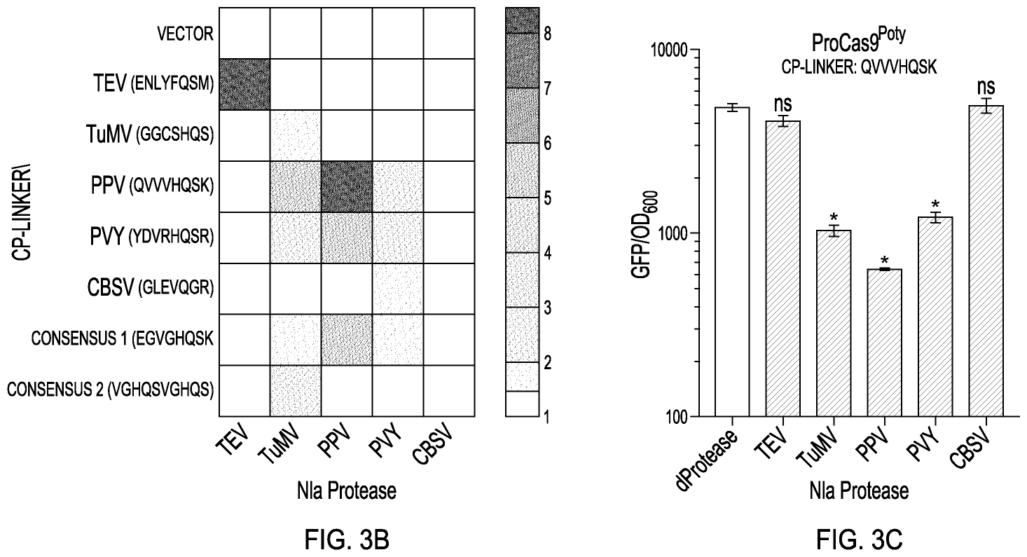

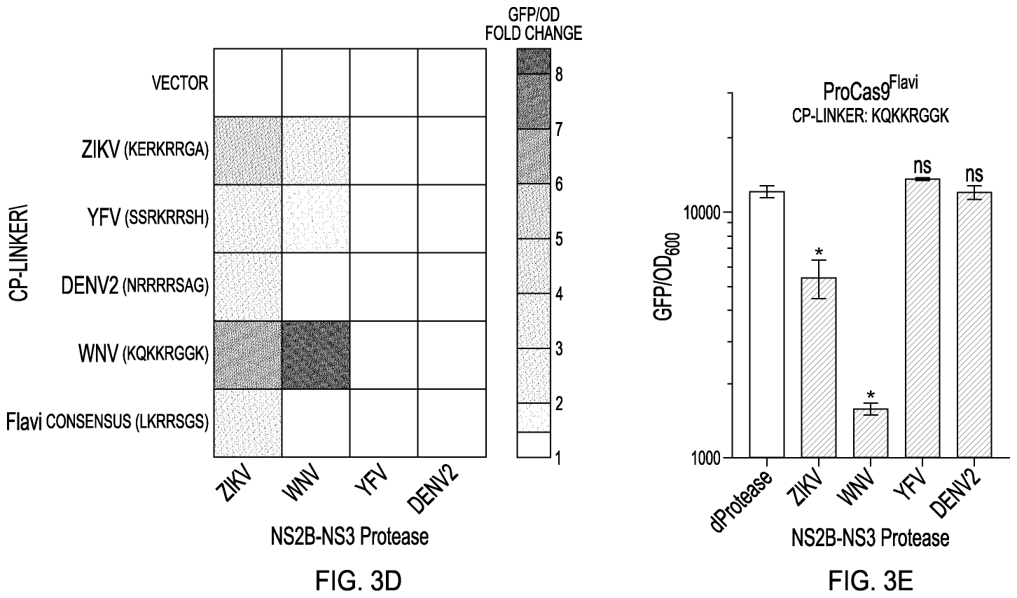

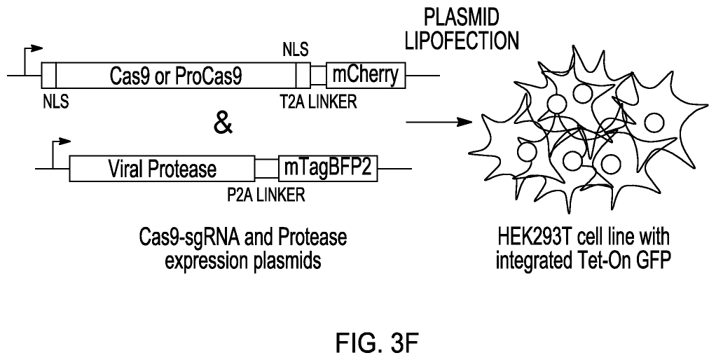

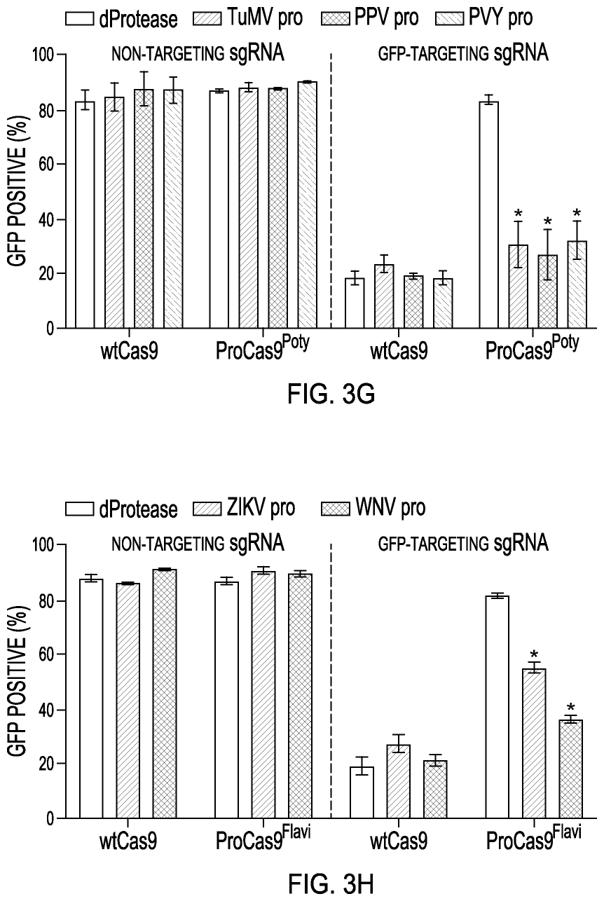

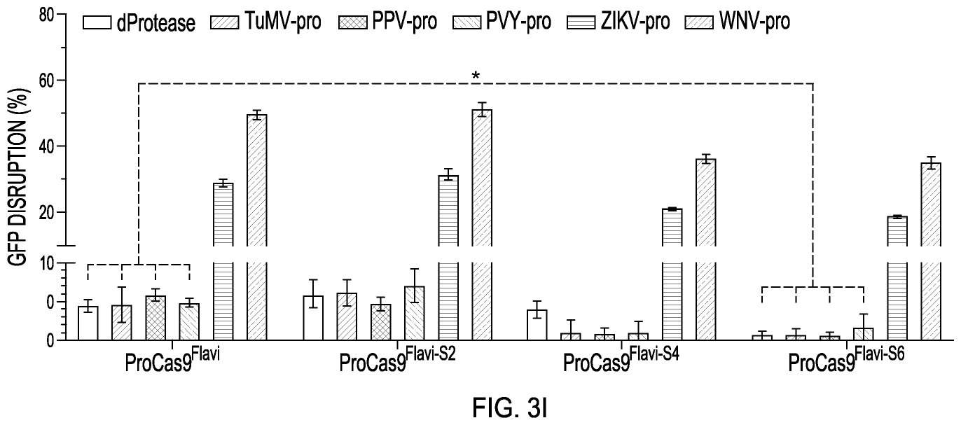

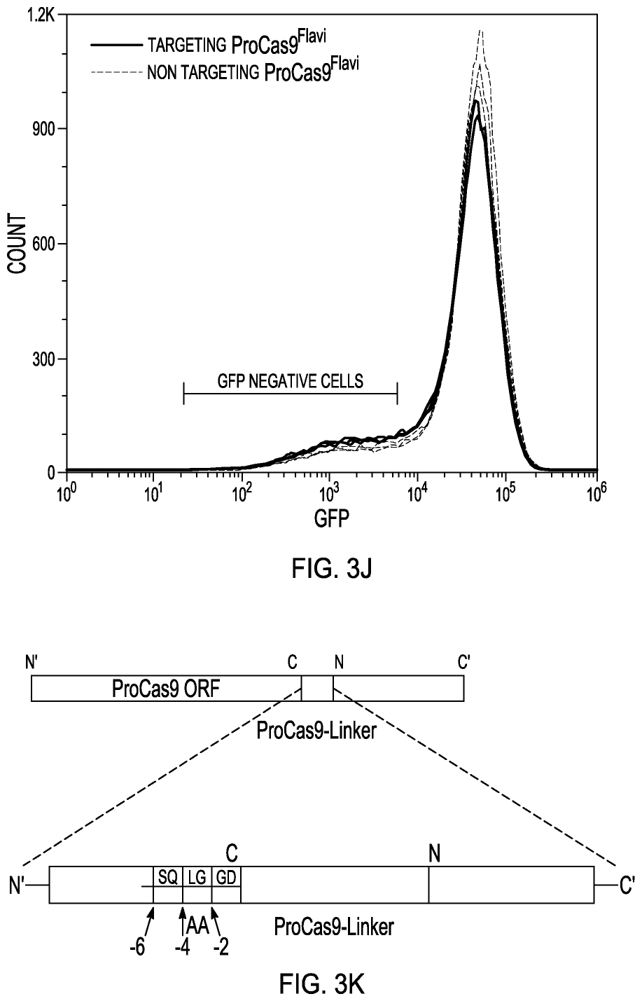

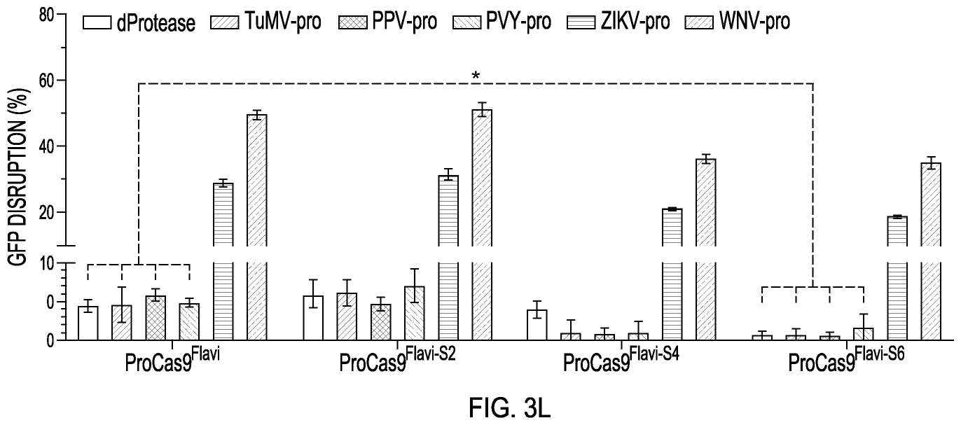

When employing the HEK-RT1 genome editing reporter assay to quantify WT Cas9 (Cas9-wt) and ProCas9 variant activity following stable genomic integration, HEK-RT1 reporter cells were transduced with the indicated Cas-wt/ProCas9 and sgRNA lentiviral vectors and selected on puromycin. A guide RNA targeting the GFP fluorescence reporter (sgGFP9) was compared to a non-targeting control (sgNT). A non-targeting control was used in all assays for normalization, in case not all non-edited cells turned GFP positive upon doxycycline treatment, though usual reporter induction rates were above 95%. GFP expression in HEK-RT1 reporter cells was induced for 24-48 hour using doxycycline (1 μg/ml; Sigma-Aldrich), at the indicated days post-editing. Percentages of GFP-positive cells were quantified by flow cytometry (Attune NxT, Thermo Fisher Scientific), routinely acquiring 10,000-30,000 events per sample. When quantifying ProCas9 activation by mTagBFP2-tagged proteases, GFP fluorescence was quantified in mTagBFP2-positive cells. In all cases, editing efficiency was reported as the difference in percentage of GFP-positive cells between samples expressing a non-targeting guide (sgNT) and samples expressing the sgGFP9 guide targeting the GFP reporter. For ProCas9 GFP disruption assays following transfection of the tested components (FIG. 3F-3H), transfection-based plasmids were designed and cloned using standard molecular biology techniques to express either ProCas9-T2A-mCherry and a single guide RNA, or the protease of interest-P2A-mTagBFP2. Transient assays were performed as follows: in triplicate the reporter cell line HEK-RT1 was seeded at 20-30 thousand cells per well into 96-well plates and transfected using 0.5 μL of Lipofectamine 2000 (Thermo Fisher Scientific), 12.5 ng of the WT Cas9 or ProCas9 plasmid and 14 ng of the Protease plasmid (2× molar ratio), following the manufacturer's protocol. Twenty-four hours later the media was changed, and doxycycline was added to induce GFP expression. 48 hours following induction the cells were gated for mCherry (WT Cas9, ProCas9) expression and analyzed using flow cytometry for GFP depletion. At least 10,000 events were collected for each sample.

### Mammalian Flow Cytometry and Fluorescence Microscopy

Flow cytometry (Attune Nxt Flow Cytometer, Thermo Fisher Scientific) was used to quantify the expression levels of fluorophores (mTagBFP2, GFP/EGFP, mCherry) as well as the percentage of transfected or transduced cells. For the HEK-RT1 genome editing reporter cell line, flow cytometry was used to quantify the percentage of GFP-negative (edited) cells, 24-48 hour after doxycycline (1 μg/mL) treatment to induce GFP expression. Phase contrast and fluorescence microscopy was carried out following standard procedures (EVOS FL Cell Imaging System, Thermo Fisher Scientific), routinely at least 48-hour post-transfection or post-transduction of target cells with fluorophore expressing constructs.

### Mammalian Immunoblotting

HEK293T (293FT; Thermo Fisher Scientific) were co-transfected with the indicated plasmids expressing Cas9-wt or ProCas9-Flavi and plasmids expressing dTEV or WNV protease. HEK293T cells were split to reach a confluency of 70%-90% at time of transfection. For transfections in 6-well plates, 1 μg Cas9-sgRNA vector and 0.75 μg protease vector (if applicable) were mixed in 0.4 mL Opti-MEM, followed by addition of 5.25 μg polyethylenimine (PEI; Polysciences #23966). After 20-30 min incubation at room temperature, the transfection reactions were dispersed over the HEK293T cells. Media was changed 12-hour post-transfection. At 36-hour post-transfection, HEK293T were washed in ice-cold PBS and scraped from the plates. Cell pellets were lysed in Laemmli buffer (62.5 mMTris-HCl pH 6.8, 10% glycerol, 2% SDS, 5% 2-mercaptoethanol). Equal amounts of protein were separated on 4%-20% Mini-PROTEAN TGX gels (Bio-Rad, #456-1095) and transferred to 0.2 μm PVDF membranes (Bio-Rad, #162-0177). Blots were blocked in 5% milk in TBST 0.1% (TBS+0.01% Tween 20) for 1 hour; all antibodies were incubated in 5% milk in TBST 0.1% at 4° C. overnight; blots were washed in TBST 0.1%. The abundance of b-actin (ACTB) was monitored to ensure equal loading. Immunoblotting was performed using the antibodies: mouse monoclonal Anti-Flag-M2 (Sigma-Aldrich, #1804, clone M2, 1:500; sigmaaldrich.com/content/dam/sigma-aldrich/docs/Sigma/Bulletin/f1804bul.pdf), mouse monoclonal C-Cas9 Anti-SpyCas9 (Sigma-Aldrich, #SAB4200751, clone 10C11-A12, 1:500; sigmaaldrich.com/content/dam/sigma-aldrich/docs/Sigma/Datasheet/10/sab4200751dat.pdf), mouse monoclonal N-Cas9 Anti-SpyCas9 (Novus Biologicals, #NBP2-36440, clone 7A9-3A3, 1:500; novusbio.com/PDFs2/NBP2-36440.pdf), HRP-conjugated mouse monoclonal Anti-Beta-Actin (Santa Cruz Biotechnology, #sc-47778 HRP, clone C4, 1:250; datasheets.scbt.com/sc-47778.pdf), and HRP-conjugated sheep Anti-Mouse (GE Healthcare Amersham ECL, #NXA931; 1:5000; see website es.vwr.com/assetsvc/asset/es_ES/id/9458958/contents). Blots were exposed using Amersham ECL Western Blotting Detection Reagent (GE Healthcare Amersham ECL, #RPN2209) and imaged using a ChemiDoc MP imaging system (Bio-Rad). Protein ladders were used as molecular weight reference (Bio-Rad, #161-0374).

### Mammalian Competitive Proliferation Assay

For assessment of CRISPR-Cas programmed cell depletion using guide RNAs targeting an essential gene (RPA1) or sgCIDEs targeting hundreds of thousands of loci within the genome, cells were stably transduced with a lentiviral vector expressing Cas9-wt (pCF226) or ProCas9Flavi (pCF730) and selected on puromycin. Subsequently, these cell lines were further stably transduced with vectors expressing various mCherry-tagged sgRNAs and analyzed as follows: 1) After mixing sgRNA expressing populations with parental cells, the fraction of mCherry-positive cells was quantified over time. Different sgRNAs targeting a neutral gene (sgOR2B6), an essential gene (sgRPA1), >100,000 genomic loci (sgCIDE) and a non-targeting control (sgNT) were compared. 2) Alternatively, the cell lines were partially transduced with lentiviral vectors expressing a GFP-tagged dTEV (pCF736) or WNV (pCF738) protease, and cell depletion quantified by flow cytometry. Depletion of protease-expressing (GFP+) cells was quantified among the sgRNA-positive (mCherry+) population.

### Statistical Analysis

Specific statistical tests used are indicated in all cases. Propagation of uncertainty was taken into consideration when reporting data and their uncertainty (standard deviation) as functions of measurement variables. Unless otherwise noted, error bars indicate the standard deviation of triplicates, and significance was assessed by comparing samples to their respective controls using unpaired, two-tailed t tests (alpha=0.05). Genome editing quantification using TIDE was carried out as recommended (Brinkman et al., 2014). In brief, indels ranging from −10 to +10 nucleotides were quantified. Parental cells were used as reference for normalization. When reporting TIDE editing efficiencies, only indels with p values <0.01 in at least one replicate were considered true.

### Data and Software Availability

To identify functional Cas9 circular permutants (Cas9-CPs), fold-changes for each dCas9-CP between pre- and post-library sorts along with significance values for each enrichment were calculated. Cas9-CP analysis scripts are available at website github.com/SavageLab/cpCas9, which is incorporated by reference herein in its entirety. All relevant sequencing data have been deposited in the National Institutes of Health (NIH) Sequencing Read Archive (SRA) at website ncbi.nlm.nih.gov/bioproject/PRJNA505363 under ID code 505363, Accession code PRJNA505363.

### Example 2: Circular Permutation of Cas9

This Example demonstrates how circular permutation can be used to re-engineer the molecular sequence of Cas9 to both better control its activity and create a more optimal DNA binding scaffold for fusion proteins.

To investigate the topological malleability of Streptococcus pyogenes Cas9 (hereafter Cas9), a random transposon insertion library was generated in vitro by adapting an engineered transposon from Jones et al. (2016) to contain a plasmid backbone, inducible promoter, and stop codon. FIG. 1I illustrates the method employed. As the original N and C termini of Cas9 are 40 to 60 angstroms apart (Anders et al., 2014), the requirements for Cas9 circular permutation are not known. Therefore, deactivated Cas9 (dCas9) was permuted using a series of linkers (GGS repeats, varying from 5 to 20 amino acids [aa]) between the original N and C termini, providing increasing steric freedom. Transposition of the engineered cassette and pooled molecular cloning yielded high insertional diversity for all libraries, as indicated by the length distributions of polymerase chain reaction (PCR) amplicons. Deep sequencing of the 20-amino acid linker library further demonstrated that about 1 of every 2 amino acids in Cas9 were observed transposition sites in the original pool, for a total of 661 circular permutant (CP) variants in the library.

Circular permutation (CP) libraries, constructed around dCas9, were screened for function in an E. coli-based repression (i.e., CRISPRi) assay targeting the expression of either RFP or GFP (Qi et al., 2013; Oakes et al., 2014, 2016). In brief, dCas9-CP libraries were targeted to repress RFP expression while GFP was used as a control for cell viability. Functional dCas9-CP library members were isolated through a sequential double-sorting procedure that enriched functional clones 100-fold to 10,000-fold (FIGS. 1B-1C). A subset of isolated clones was plated for each of the libraries (i.e., 5, 10, 15 and 20 amino acid linkers) and sequenced. For the 5 and 10 amino acid linker-library only a minimal number of CPs around the original termini was observed. However, the 15 and 20 amino acid linker libraries yielded a number of CP variants and isolated clones were found to be highly functional in bacterial CRISPRi assays (FIG. 1E; Table 3).

TABLE 3
Cas9 Circular Permutants

| | Domain | Original | New Start |
|---|---|---|---|
| | at CP | Sequence | Site (aa |
| Name | Site | at CP site | position) |
| Cas9-CP¹⁸¹ | Helical-II | PDNSD\|VDKLF | 181 |
| | | (SEQ ID NO: 67) | |
| Cas9-CP¹⁹⁹ | Helical-II | QLFEE\|NPINA | 199 |
| | | (SEQ ID NO: 68) | |
| Cas9-CP²³⁰ | Helical-II | LIAQL\|PGEKK | 230 |
| | | (SEQ ID NO: 69) | |
| Cas9-CP²⁷⁰ | Helical-II | QLSKD\|TYDDD | 270 |
| | | (SEQ ID NO: 70) | |
| Cas9-CP³¹⁰ | Helical-II | ILRVN\|TEITK | 310 |
| | | (SEQ ID NO: 71) | |
| Cas9-CP¹⁰¹⁰ | RuvC-III | ESEFV\|YGDYK | 1010 |
| | | (SEQ ID NO: 72) | |
| Cas9-CP¹⁰¹⁶ | RuvC-III | GDYKV\|YDVRK | 1016 |
| | | (SEQ ID NO: 73) | |
| Cas9-CP¹⁰²³ | RuvC-III | VRKMI\|AKSEQ | 1023 |
| | | (SEQ ID NO: 74) | |
| Cas9-CP¹⁰²⁹ | RuvC-III | KSEQE\|IGKAT | 1029 |
| | | (SEQ ID NO: 75) | |
| Cas9-CP¹⁰⁴¹ | RuvC-III | YFFYS\|NIMNF | 1041 |
| | | (SEQ ID NO: 76) | |
| Cas9-CP¹²⁴⁷ | CTD | YEKLK\|GSPED | 1247 |
| | | (SEQ ID NO: 77) | |
| Cas9-CP¹²⁴⁹ | CTD | KLKGS\|PEDNE | 1249 |
| | | (SEQ ID NO: 78) | |
| Cas9-CP¹²⁸² | CTD | SKRVI\|LADAN | 1282 |
| | | (SEQ ID NO: 79) | |

Nomenclature and local sequence of select Cas9 circular permutants (Cas9-CPs). The superscript in the name indicates the original amino acid (aa) in Streptococcus pyogenes Cas9 that now serves as the new N-terminus.

The majority of functional clones were found in the 20-amino acid linker library. Deep sequencing of this library was performed to generate an enrichment profile of permutation across Cas9. Seventy-seven sites were identified as highly enriched (>100-fold) following the double sorting procedure (FIG. 1C). Notably, all confirmed hits (FIG. 1E) and internal controls fell within this group. Mapping the observed sites onto the protein sequence (FIG. 1D) revealed three hotspots of CPs (all numbering based on Streptococcus pyogenes Cas9 protein sequence): in the Helical-II (aa 178-314), in the RuvC-III (aa 940-1150) and in the CTD (aa 1240-1299) domains (FIG. 1D). These hotspots qualitatively correspond with those that the inventors have previously identified for Cas9 domain insertion (Oakes et al., 2016), indicating that the underlying structural and biochemical constraints may be similar. Intriguingly, among the newly discovered termini, a number are in direct contact (less than 5 angstroms) with the non-target strand, yielding Cas9-CPs containing ideal fusion points for protein domains to modify the isolated single-strand that heretofore required long linkers to gain such access (i.e., base editors) (Gaudelli et al., 2017; Guilinger et al., 2014; Komor et al., 2016; Tsai et al., 2014).

The isolated Cas9-CPs were next tested for their cleavage activity relative to wild-type (WT) Cas9. Briefly, two variants from each of the three hotspots (specifically, CP sites 199, 230, 1010, 1029, 1249, and 1282) were constructed with a 20-amino acid linker between the original N and C termini and recoded with functional nuclease active sites (Table 3). Testing of these constructs for genomic cleavage and killing activity in E. coli demonstrated that all possessed similar activity as WT Cas9 (FIG. 1F). To assess how well these findings extrapolate to mammalian systems, a rapid human genome editing reporter assay was established with a quantitative fluorescence-based readout of target disruption activity and editing efficiency (Example 1). When compared relative to WT Cas9 in this assay, the Cas9-CPs showed surprisingly high genome editing efficiency (FIG. 1G). While more variation was observed than in the E. coli-based experiments, four tested CP variants (CP199, CP1029, CP1249, CP1282) showed 80% or more of WT activity. Overall, these results demonstrate that Cas9 can be circularly permuted to create novel proteins that upon cleavage and/or folding can maintain wild type like levels of DNA binding and cleavage activity.

### Example 3: Cas9-CP Activity can be Regulated by Proteolytic Cleavage

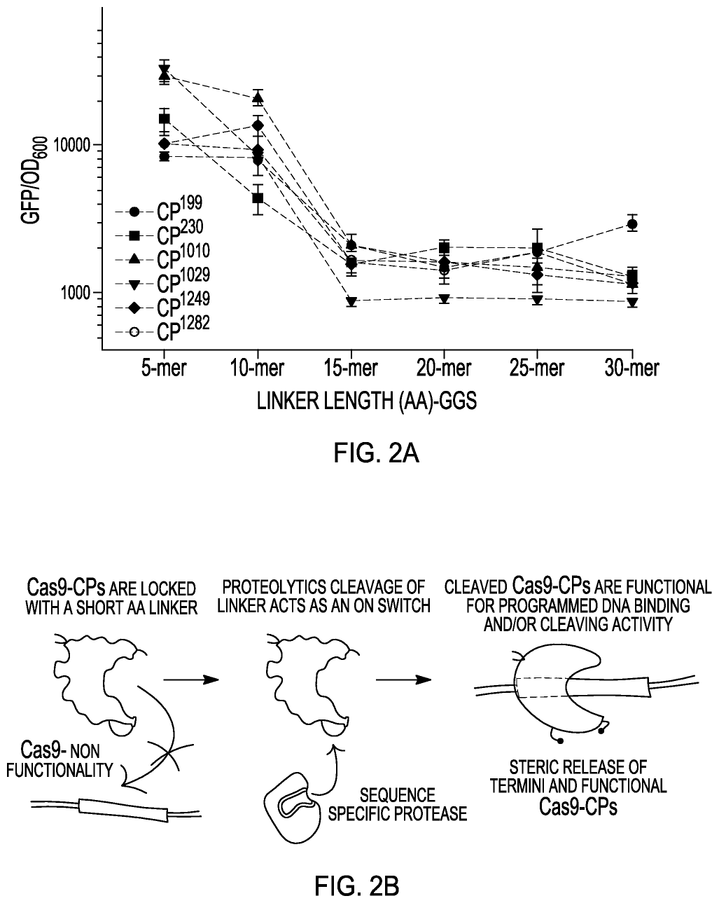

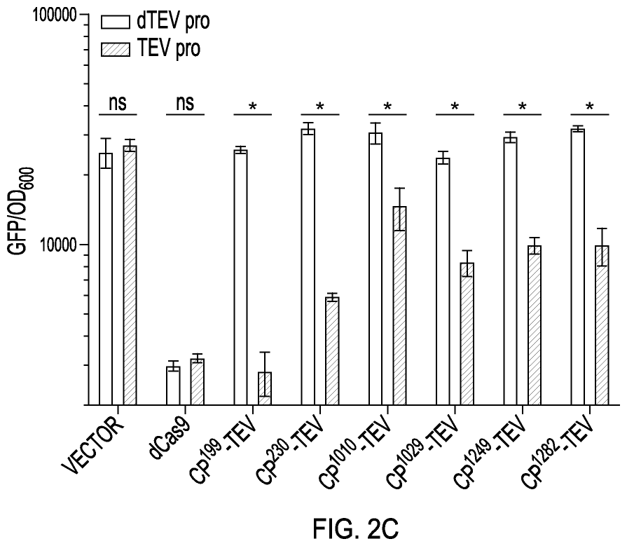

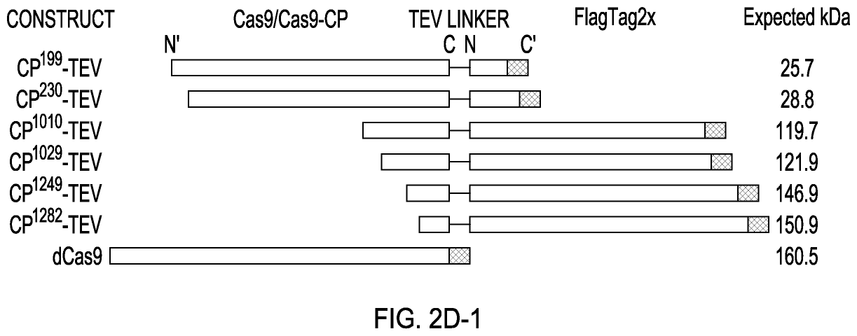

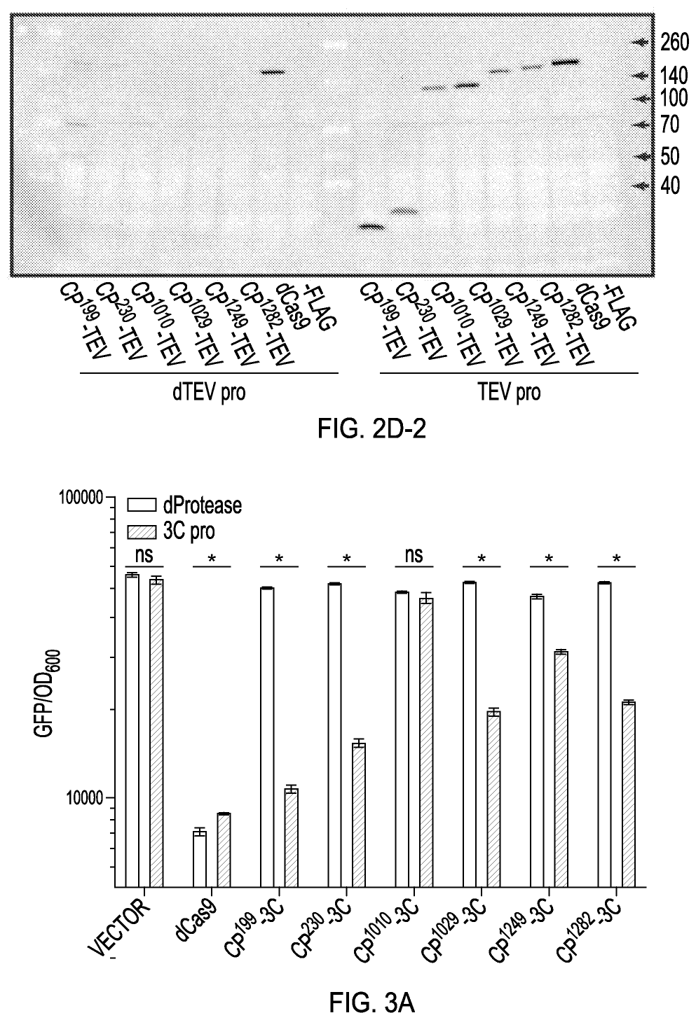

Characterization of the libraries described above revealed that circular permutation is highly sensitive to the linker length connecting the original N and C terminus. PCR analysis of pooled libraries indicated that a linker length of 5 aa or 10 aa was not sufficient to generate Cas9-CP diversity. Conversely, libraries of 15 or 20 aa linkers qualitatively possessed extensive permutable diversity. Therefore, the inventors decided to test the importance of linker length on confirmed sites identified above (FIG. 1E). The same six Cas9-CPs (i.e., Cas9-CP199 through Cas9-CP1282) were cloned with linkers (GGS repeats) from 5 to 30 aa and tested for repression of GFP in an E. coli-based CRISPRi assay (FIG. 2A).

In agreement with the pooled libraries, we found that all Cas9-CPs with linkers of 5 and 10 aa in length were markedly disrupted in activity, while those with longer linkers were active. Notably, activity did not increase with linker length beyond 15 aa (FIG. 2A).

The sensitivity of CPs to linker length led us to hypothesize that Cas9-CPs could be made into “caged” variants that could switch from an inactive form to an active one upon post-translational modification (FIG. 2B). It has previously been observed that circularly permuted proteins can be sensitive to the length of the linker between their old N and C termini (Yu and Lutz, 2011). This requirement has been exploited to create zymogen pro-enzymes by replacing the linker with a site-specific protease sequence, such that proteolytic cleavage converts a short linker into an effectively infinite linker with concomitant turn-on in protein activity. Although potentially useful for applications in biosensing (e.g., pathogen or cancer detection) existing sensors were constructed around either RNase A (Johnson et al., 2006; Plainkum et al., 2003) or barnase (Butler et al., 2009) and possess limited in vivo potential because of their inherent nonspecific, toxic activity.

To test the possibility of turning Cas9-CPs into activatable switches using a well-studied protease, the six representative CP variants were engineered to include the 7-amino acid cleavage site (ENLYFQ/S) of the tobacco etch virus (TEV) nuclear inclusion antigen (NIa) protease as the linker sequence (Seon Han et al., 2013). This 7-amino acid linker was able to fully disrupt Cas9-CP activity in the E. coli CRISPRi GFP repression assay (FIG. 2C). Upon addition of a fully active TEV protease, activity was restored to a varying degree in all six Cas9-CPTEV constructs. Notably, Cas9-CP199 switched from completely off to fully on (FIG. 2C) and performed consistently over a 20-hr time course. This switch behaved well across the population in single cell assays and did not activate when a TEV catalytic triad mutant, C151A, was expressed (dTEV). Finally, to verify if TEV is cleaving Cas9-CPs at the CP linker, cells were recovered from the endpoint of the CRISPRi assay (FIG. 2C) for western blot analysis against a 2× Flag-tag cloned onto the C terminus of the protein. As shown in FIG. 2D, when an active TEV protease was present, products were observed corresponding to the size of the C-terminal circularly permuted fragment.

### Example 4: Regulating Caged Cas9's with Site-Specific Proteases

This Example illustrates that the uncaging mechanism for releasing Cas9-CP activities can be used with a variety of proteases.

The human rhinovirus 3C is responsible for about 30% of cases of the common cold and contains a well-studied protease, human rhinovirus 3C protease (3Cpro), unrelated to that from tobacco etch virus (TEV) (Skern, 2013). The eight-amino acid linker with the TEV recognition site was replaced in the six Cas9-CPs with the linker sequence with the for 3Cpro (LEVLFQ/GP SEQ ID NO:87). The six Cas9-CPs with the 3Cpro linker were then tested for bacterial CRISPRi activity with and without active protease.

Protease-dependent activation of Cas9-CPs was observed, with varying amounts of turn-on in activity, thus demonstrating that the deactivation-reactivation mechanism can be extended to other proteases (FIG. 3A). The Cas9-CP199 with the 3Cpro cleavage site exhibited the largest difference when released by the human rhinovirus 3C protease. Hence, the Cas9-CP199 with the greatest response was used for all experiments described below.

Next, the protease sensing Cas9-CPs (hereafter ProCas9s) were tested on agriculturally and medically relevant viruses.

The Potyvirus proteases from turnip mosaic virus (TuMV), plum pox virus (PPV), potato virus Y (PVY), and cassava brown streak virus (CBSV) were tested, all of which are plant viruses responsible for significant crop losses each year (Seon Han et al., 2013; Tomlinson et al., 2018). The nuclear inclusion antigen (NIa) protease genes from these viruses were also cloned.

These protease constructs were evaluated for co-expression in conjunction with ProCas9s having linkers from a set of proteases of a medically important Flavivirus genus. Briefly, the capsid protein C cleavage sequences from Zika virus (ZIKV), West Nile virus (WNV, Kunjin strain), Dengue virus 2 (DENV2), and yellow fever virus (YFV) (Bera et al., 2007; Kummerer et al., 2013) were used as the CP linker sequence to generate a set of flavivirus-specific ProCas9s. In the viral life cycle, these cleavage sequences are cut by the NS2B-NS3 protease from the respective virus to mature the polyprotein (Kummerer et al., 2013).

Cognate protease cleavage sites (STAR Methods) were used as the CP linker in Cas9-CP199, yielding the respective ProCas9s that were systematically tested against all co-expressed N1a proteases. The following Table 4 shows sequences for the protease-specific linkers used with the Cas9-CP199 protein to provide protease-activated Cas9 activity by the Zika virus (ZIKV), yellow fever virus (YFV), Dengue virus 2 (DENV2), West Nile virus (WNV, Kunjin strain), and Flavi virus (consensus).

TABLE 4
Protease-Specific Linker Sequences

| Protease | Linker | Linker |
|---|---|---|
| | Sequence | SEQ ID NO: |
| West Nile virus (WNV, | KQKKRGGK | SEQ ID NO: 80 |
| Kunjin strain) | | |
| Human rhinovirus 3C | LEVLFQGP | SEQ ID NO: 87 |
| protease (3Cpro) | | |
| Zika virus (ZIKA) | KERKRRGA | SEQ ID NO: 88 |
| Yellow fever virus | SSRKRRSH | SEQ ID NO: 89 |
| (YFV) | | |
| Dengue virus 2 (DENV2) | NRRRRSAG | SEQ ID NO: 90 |
| Flavi virus | LKRRSGS | SEQ ID NO: 91 |
| Plum pox virus (PPV) | QVVVHQSK | SEQ ID NO: 93 |

CRISPRi experiments revealed a general trend of proteases activating their respective ProCas9 (FIG. 3B-3D). In addition, the plum pox virus (PPV) linker (QVVVHQ/SK; SEQ ID NO: 92) enabled a ProCas9 response to three different N1a proteases with specificity distinct from TEV (FIG. 3B-3C). This variant was called ProCas9Poty for a Cas9 that can recognize and respond to a number of agriculturally important Potyvirus proteases.

Screening of these Flavivirus ProCas9 variants against their cognate proteases revealed a variant—hereafter called Pro-Cas9Flavi—that possesses a WNV linker sequence (KQKKR/GGK, SEQ ID NO:80) and was activated by NS2B-NS3 proteases from both Zika and WNV (FIGS. 3D-3E). No activation was observed with the CBSV, DENV2, or YFV proteases; this may be due to non-optimal CP linkers, poor expression of the cognate proteases, or a steric hindrance blocking the protease from reaching the CP linker site.

Next, the function of ProCas9s was validated and optimized in eukaryotic cells using a transient transfection system in the HEK293T-based GFP disruption assay (FIGS. 3G-3H). Expression of either ProCas9Poty or ProCas9Flavi resulted in GFP disruption only in the presence of the active proteases (FIGS. 3G-3H).

A small amount of leaky activation (about 5%) was also observed in the absence of protease activity, so the distance between the original N and C termini was tested by progressively shortening by 2, 4, or 6 amino acids to evaluate whether such shortening would reduce unwanted background activity. While removing two amino acids from ProCas9Flavi had no apparent effect, removing six amino acids (ProCas9Flavi-S6) significantly reduced activity levels for nonactive or non-corresponding active proteases while still enabling a response, albeit weaker, to both ZIKV and WNV (corresponding) proteases (FIG. 3I). Thus, linker “tightening” optimization provides an additional safety mechanism, allowing a ProCas9 to exist in cells with little risk of untriggered genome cleavage activity.

### Example 5: ProCas9 can be Stably Integrated into Mammalian Genomes without Leaky Activity

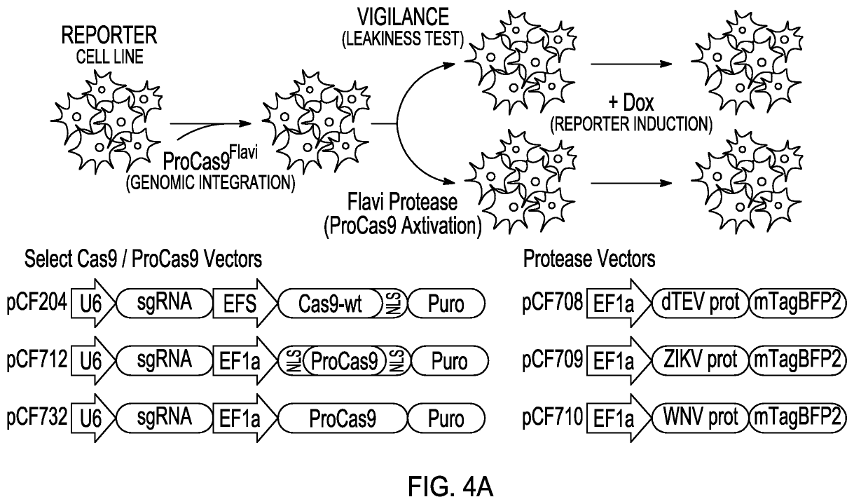

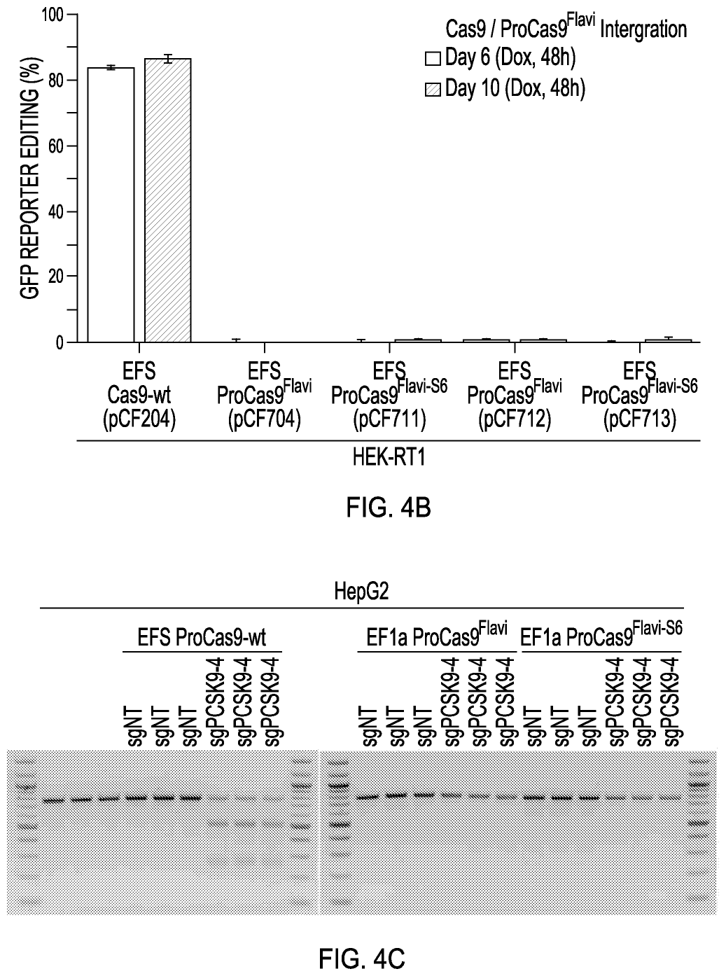

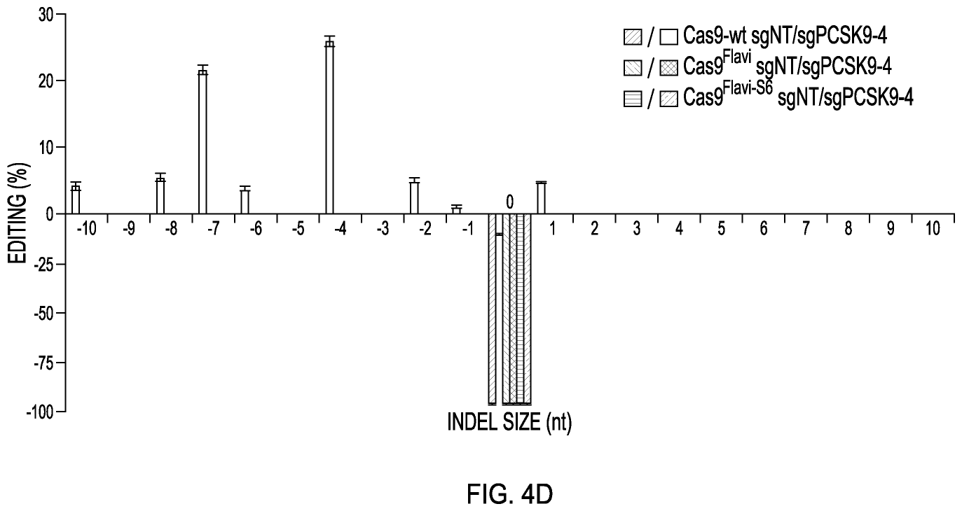

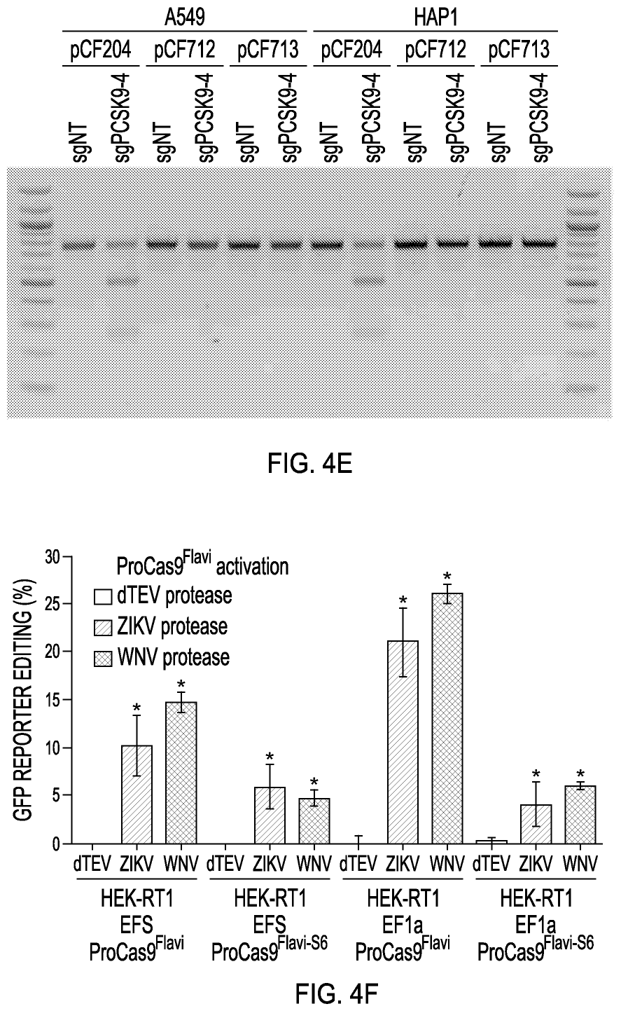

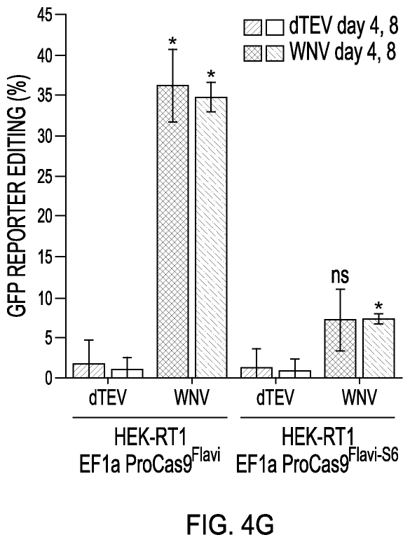

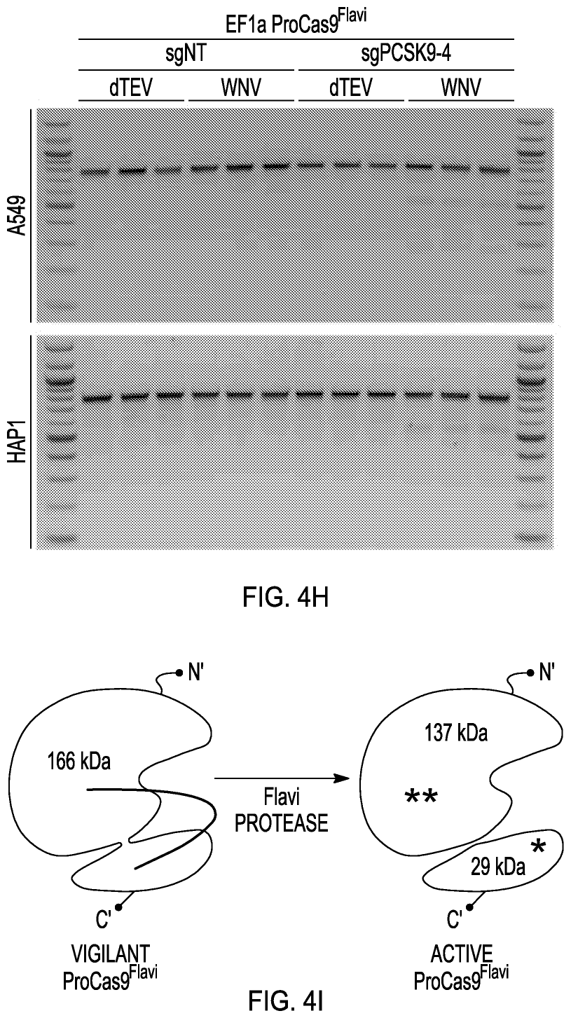

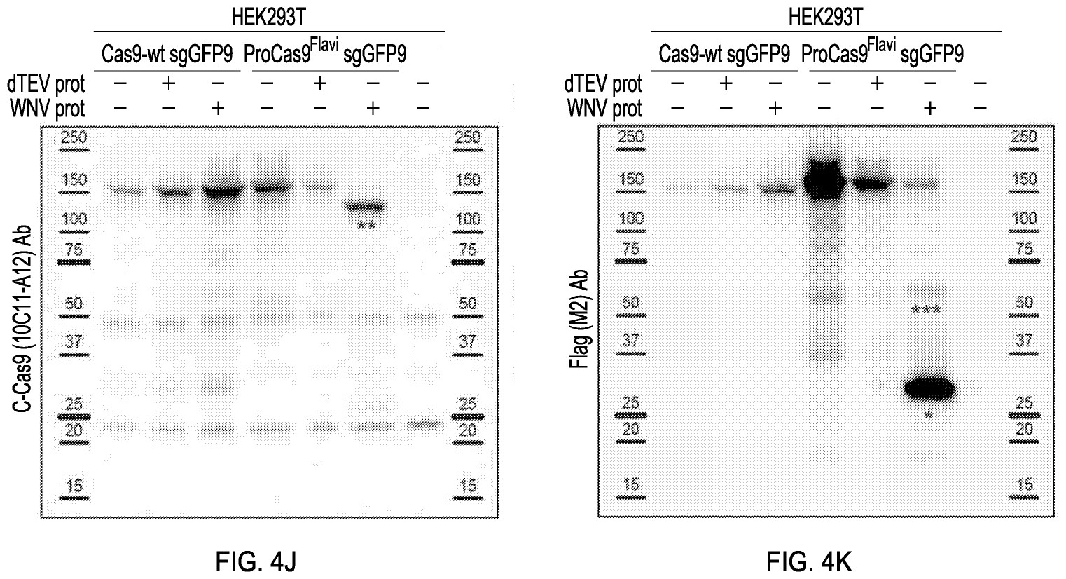

A prerequisite for using activatable genome editors in sensing or molecular recording applications is that they possess low background activity under stable expression conditions. To confirm that ProCas9s function accordingly, lentiviral vectors were built that expressed ProCas9 from either a weak EF1a core promoter (EFS) or strong full-length EF1a promoter, along with single guide RNAs (sgRNAs) driven from a U6 promoter. The lentiviral vectors were tested for ProCas9Flavi and ProCas9Flavi-S6 activity in HEK-RT1 reporter cells (FIG. 4A).

When measured 6 to 10 days post-transduction, none of the four tested ProCas9 constructs showed any background activity (FIG. 4B), indicating that the systems are not leaky. To further confirm these findings at an endogenous locus, the non-essential PCSK9 locus was targeted in the hepatocellular carcinoma cell line HepG2. Eight days after stable transduction with ProCas9Flavi, ProCas9Flavi-S6 or WT Cas9 PCSK9 editing efficiency was assessed by T7 endonuclease 1 (T7E1) assay (FIG. 4C). While WT Cas9 showed high levels of editing, no leakiness was observed with any of the ProCas9 constructs.

TIDE analysis (Brinkman et al., 2014) was used to quantify editing outcome (FIG. 4D), revealing 71.1% editing with WT Cas9 (11.6% non-edited, 17.3% undetected in the −10- to +10-nt indel range) and confirming the absence of background editing with the ProCas9 constructs. Finally, editing at the PCSK9 locus was also tested in the lung carcinoma cell line A549 and the haploid chronic myeloid leukemia derived line HAP1, two cell lines often used for Flavivirus assays (FIG. 4E). Again, the ProCas9 constructs displayed no background activity.

### Example 6: Genomic ProCas9 can be Activated by Flavivirus Proteases to Induce Target Editing

An activatable switch for molecular sensing must display repeatable induction upon stimulation. In an initial test, HEK-RT1 reporter lines (FIG. 4B) containing stably integrated Flavivirus ProCas9s were transiently transfected with vectors expressing dTEV, ZIKV, and WNV proteases, each tagged with mTagBFP2 to enable tracking of activity (FIG. 4A). Two days post-transfection, the GFP reporter was induced by doxycycline treatment for 24 hours and quantified for editing efficiency by flow cytometry in mTagBFP2-positive cells. While dTEV protease expression did not lead to genome editing in any reporter cell line, both ZIKV and WNV protease activity led to genome editing, especially with the ProCas9Flavi system. The ProCas9Flavi system driven by the stronger EF1a promoter showed the highest genome editing efficiency (FIG. 4F). Together, this indicates that ProCas9 constructs can sense and record Flavivirus protease activity associated with transient expression.

To mimic a viral infection more closely, we next evaluated whether a stably integrated viral vector expressing Flavivirus proteases could also activate ProCas9Flavi enzymes. To generate viral particles, HEK293T packaging cell lines were transfected with dTEV, ZIKV, or WNV protease-encoding lentiviral vectors. Expressing the NS2B-NS3 or NS3 protease is known to be toxic (Ramanathan et al., 2006), and a similar effect was observed with ZIKV and WNV proteases, which led to reduced viral titers and target cell transduction efficiency. Nevertheless, we were able to stably transduce the HEK-RT1-ProCas9 reporter cell lines with protease constructs and followed the effects of dTEV, ZIKV, and WNV protease expression (FIG. 4F). While the dTEV protease did not lead to any editing, both the ZIKV and WNV proteases induced genome editing in all four tested ProCas9 lines, with the strongest effect (over 25% editing) again observed with the EF1a-ProCas9Flavi system induced by the WNV protease.

To assess the dynamic range of ProCas9Flavi induction, the above experiments were repeated out to 8 days (FIG. 4G). Here, stable expression of the WNV protease led to about 35% genome editing when sensed by the EF1a-ProCas9Flavi system. In further tests, an EF1a-ProCas9Flavi construct was tested that did not contain any nuclear localization sequence (NLS). The inventors observed that WNV protease-mediated induction was reduced compared to NLS containing constructs. These results were qualitatively confirmed, based on mTagBFP2-positive cells expressing the protease, using a T7E1 assay.

As with background activity testing, the activation of ProCas9s by proteases was further validated by targeting the endogenous PCSK9 locus (FIG. 4H). Qualitative T7E1-based analysis showed that while no genome editing was observed with a non-targeting guide, the EF1a-ProCas9Flavi system equipped with a guide targeting PCSK9 (sgPCSK9-4) showed clear genome editing in the presence of WNV protease, but not a negative control (dTEV). Together with the absence of leakiness, this clearly demonstrates that ProCas9 can be stably integrated into mammalian genomes to sense, record and respond to endogenous or exogenous protease activity.

### Example 7: Mechanism of ProCas9 Activation in Mammalian Cells

Conceptually, the underlying idea of ProCas9s is that they are present in cells in an inactive, or “vigilant,” state due to the linker sterically inhibiting activity (FIG. 4I). The presence of a cognate protease recognizing the peptide linker relieves inhibition through target cleavage, and leads to an “active” ProCas9 composed of two distinct subunits. To explore this hypothesis, HEK239T cells were co-transfected with vectors expressing either Cas9 WT or ProCas9Flavi and the dTEV or WNV protease. Immunoblotting with antibodies for the full-length Cas9 WT and vigilant ProCas9Flavi—as well as both the small (about 29 kDa) and large (about 137 kDa) subunit of active ProCas9Flavi—showed that Cas9 WT and ProCas9Flavi are expressed to comparable extents in the absence of a cognate protease (FIG. 4J-4K). In the presence of the WNV protease, however, the vast majority of vigilant ProCas9Flavi was activated and observed as two distinct subunits, confirming the hypothesized mechanism.

### Example 8: Rapid CRISPR-Cas-Controlled Cell Depletion

A molecular sensor, such as ProCas9, could actuate many types of outputs. One unique effect would be to induce cell death upon sensing viral infection, as a form of altruistic defense. Since activated ProCas9 is capable of inducing DNA double-strand breaks, we sought to identify sgRNAs that could induce rapid cell death. As Flaviviruses replicate rapidly upon target cell infection, such sgRNAs would have to kill their host cells in less time. Targeting essential genes such as the single-stranded DNA binding protein RPA1, which is involved in DNA replication, could be one option. Alternatively, targeting highly repetitive sequences within a cell's genome to induce massive DNA damage and cellular toxicity could be another avenue. Indeed, sgRNAs targeting even only moderately amplified loci have been shown to lead to cell depletion under certain conditions (Wang et al., 2015), independent of whether the sgRNA targets a gene or intergenic region. While these effects have been observed over long assay periods, targeting highly repetitive sequences might provide sufficient DNA damage to trigger rapid cell death.

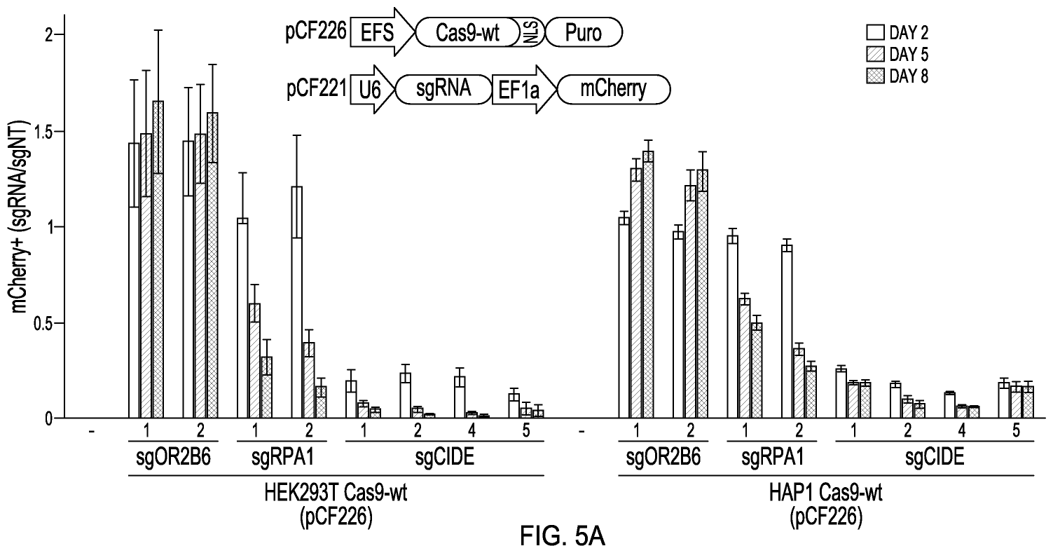

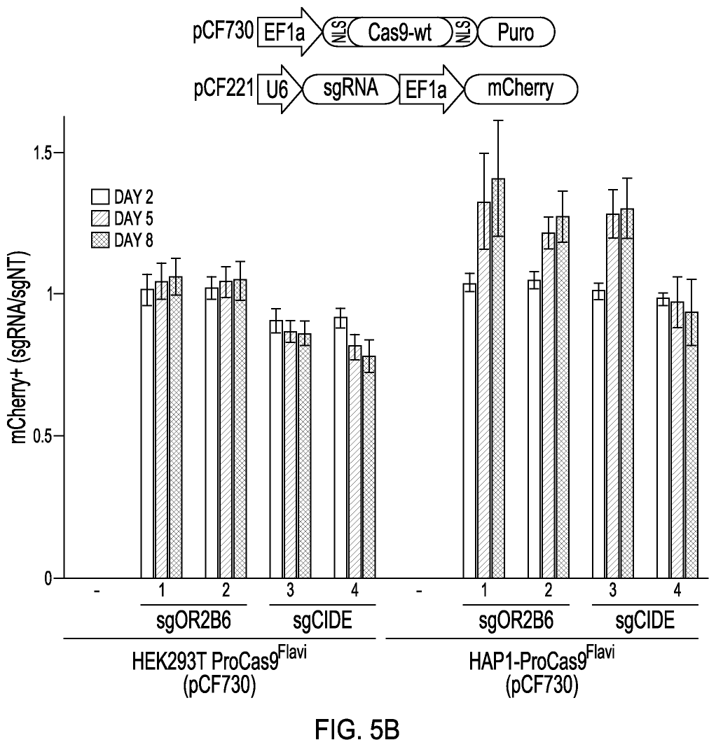

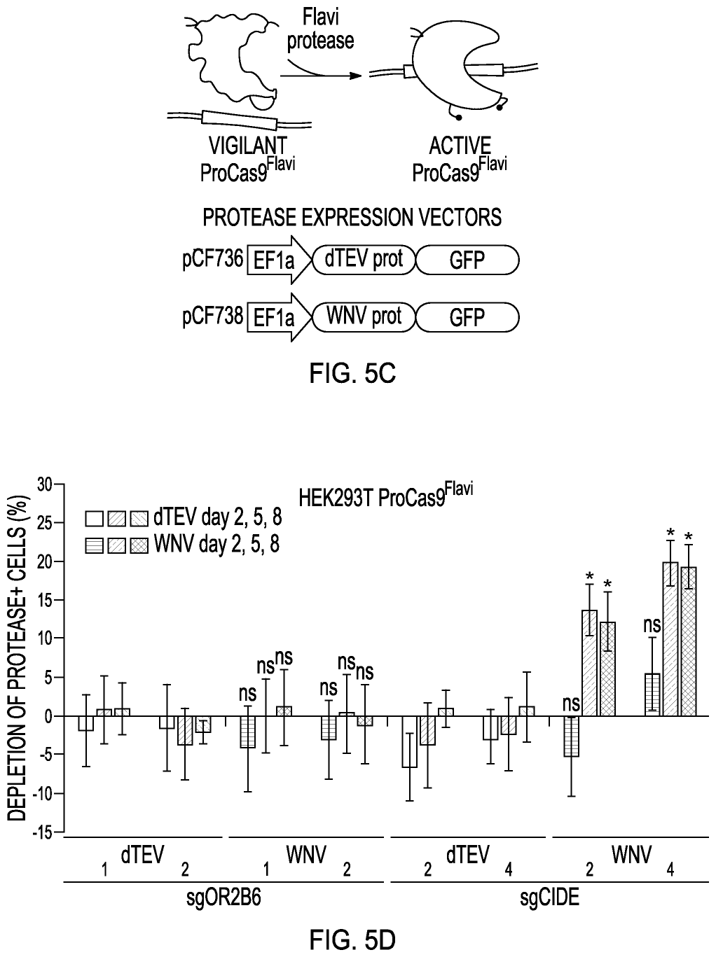

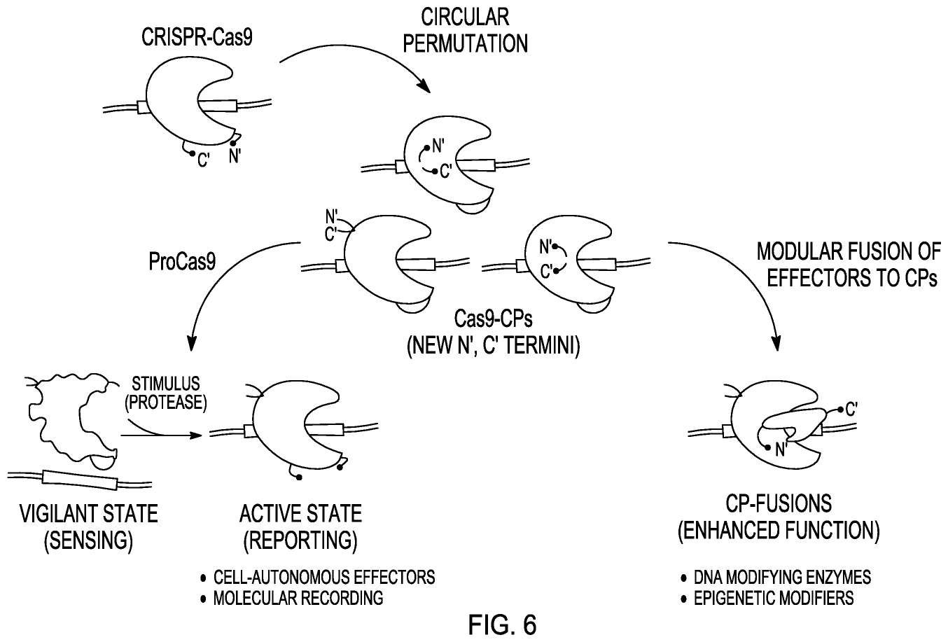

To compare the two strategies, both HEK293T and HAP1 cells were stably transduced to express WT Cas9 and an sgRNA coupled to an mCherry fluorescence marker (FIG. 5A). The effect of guide RNA expression on cell viability was assessed using a competitive proliferation assay in which cells expressing a specific sgRNA were mixed with parental cells expressing only Cas9 WT, and the mCherry-positive population was followed over time. Negative control guides targeting an olfactory receptor gene (sgOR2B6-1, sgOR2B6-2) showed no depletion. Guide RNAs targeting the essential RPA1 gene depleted over the eight-day assay period. To potentially accelerate depletion, several sgRNAs were also designed and tested, where the sgRNAs targeted repetitive sequences in the human genome (about 125,000-300,000 target loci each, STAR Methods), which could cause CRISPR Cas induced death by editing or “CIDE.” Indeed, CIDE guide RNAs (sgCIDE-1, sgCIDE-2, sgCIDE-4, sgCIDE-5) led to rapid elimination of the mCherry-positive population (FIG. 5A) and show promise as a simple genetic output module for an altruistic defense system based on CRISPR-Cas-mediated cell death.

### Example 9: Genomic ProCas9 can Sense Flavivirus Proteases and Mount an Altruistic Defense

Cas-induced death by editing or ‘CIDE, as an output constrains the performance of ProCas9. The system remains off to minimize genomic damage yet is vigilant to respond to a stimulus. To develop this protease-induced altruistic defense platform, stable expression of the best CIDE guide RNAs (sgCIDE-2, sgCIDE-4) was assessed in conjunction with a genomically integrated ProCas9Flavi cassette to determine cell viability in the absence of a stimulus (FIG. 5B). Competitive proliferation assays analogous to the ones run with WT Cas9 showed that in the presence of ProCas9Flavi only minimal amounts of cell depletion were observed. Induction of this stably integrated altruistic defense system was then tested by Flavivirus proteases. Using the same cell lines (expressing ProCas9Flavi) as above, stable transduction was observed with vectors expressing either a control (dTEV) or Flavivirus (WNV) protease led to specific cell depletion only when both the WNV protease was present and the system was programmed with one of the two CIDE sgRNAs (FIGS. 5C-5D). Hence, these results confirmed that the Flavivirus ProCas9 system can be stably integrated into the genome of a host cell to detect predefined protease activity and mount a programmed defense, only in the presence of a specific stimulus of interest.

### Example 10: Guide RNAs that Target Repetitive Genomic DNA

To investigate the ability of CRISPR-Cas9 to eliminate glioblastoma cells through targeting of repetitive sequence elements in their genomes, ten of the most common repetitive single-guide RNA (sgRNA) target loci in the human genome were identified as 20-mers with adjacent 5′-NGG-3′ protospacer adjacent motifs (PAMs). Single guide RNAs (referred to as sgCIDE RNAs for CRISPR-Cas induced death by editing) were designed to target repetitive or highly repetitive sequences in the target genome. The number of off-target sites was further determined with a Hamming distance (mismatches) of up to three and allowing for NGG or NAG PAMs. Specific examples include, but are not limited to, the following sgCIDE RNAs targeting the human and/or mouse genome shown in Table 2.

TABLE 2
sgCIDE RNA Sequences

| | | SEQ |
|---|---|---|
| | | ID |
| Name | Sequence | NO: |
| sgCIDE-1 | TGTAATCCCAGCACTTTGGG | 1 |
| sgCIDE-2 | TCCCAAAGTGCTGGGATTAC | 2 |
| sgCiDE-3 | GCCTGTAATCCCAGCACTTT | 3 |
| sgCIDE-4 | CGCCTGTAATCCCAGCACTT | 4 |
| sgCIDE-5 | CCTCGGCCTCCCAAAGTGCT | 5 |
| sgCIDE-6 | CCCAGCACTTTGGGAGGCCG | 6 |
| sgCIDE-7 | CTCCCAAAGTGCTGGGATTA | 7 |
| sgCIDE-8 | CTGTAATCCCAGCACTTTGG | 8 |
| sgCIDE-9 | TCCCAGCACTTTGGGAGGCC | 9 |
| sgCIDE-10 | TTCTCCTGCCTCAGCCTCCC | 10 |
| sgCIDE-21 | AGTGAGTTCCAGGACAGCCA | 11 |
| sgCIDE-22 | TTGTTCCACCTATAGGGTTG | 12 |
| sgCIDE-23 | CTTTCTCTAGCTCCTCCATT | 13 |
| SgCIDE-24 | CCCAATGGAGGAGCTAGAGA | 14 |
| sgCIDE-31 | CCATTCTGACTGGTGTGAGA | 15 |
| sgCIDE-32 | GAAGTCCTAGCCAGAGCAAT | 16 |
| sgCIDE-33 | ATTGCTCTGGCTAGGACTTC | 17 |
| sgCIDE-34 | GTCTCCCACTATTATTGTGT | 18 |
| sgCIDE-35 | TTGAATCTGTAGATTGCTTT | 19 |
| sgCIDE-36 | CCTCCCAAGTGCTGGGATTA | 20 |
| sgCIDE-41 | AAGAAAGAAAGAAAGAAAGA | 21 |
| sgCIDE-42 | GAGAGAGAGAGAGAGAGAGA | 22 |
| sgCIDE-43 | AGGAAGGAAGGAAGGAAGGA | 23 |
| sgCIDE-44 | TAGATAGATAGATAGATAGA | 24 |
| sgCIDE-45 | CACACACACACACACACACA | 25 |
| sgCIDE-46 | TGGATGGATGGATGGATGGA | 26 |
| sgCIDE-Alu | AGTAATCCCAGCACTTTGGG | 27 |
| sgCIDE-SINE-B2 | GGGCTGGAGAGATGGCTCAG | 28 |
| sgNT-1 | GGCCAAACGTGCCCTGACGG | 29 |
| sgNT-2 | GCGATGGGGGGGTGGGTAGC | 30 |
| sgNT-3 | GACGACTAGTTAGGCGTGTA | 31 |
| sgOR2B6-1 | CATTATTCTAGTGTCACGCC | 32 |
| sgOR2B6-2 | GGGTATGAAGTTTGGTGTCC | 33 |
| sgOR2B6-3 | AATGGTCAGATTGCCAAAGA | 34 |
| sgRPAl-1 | ACAAAAGTCAGATCCGTACC | 35 |
| sgRPAl-2 | TACCTGGAGCAACTCCCGAG | 36 |
| sgRPAl-3 | ACTTTCGTCAACCAGTTCTA | 37 |

The sgCIDEs examined could target about 3,000-300,000 sites per haploid genome. For example, as shown in Table 5 sgCIDEs with SEQ ID Nos: 1-3 could target approximately up to 300,000 sites per haploid genome.

TABLE 5
Genomic Target Count of Select Highly
Repetitive sgCIDEs

| | | No. of |
|---|---|---|
| Name | Sequence | Target Loci |
| sgCIDE-1 | TGTAATCCCAGCACTTTGGG | 288,646 |
| | (SEQ ID NO: 1) | |
| sgCIDE-2 | TCCCAAAGTGCTGGGATTAC | 285,062 |
| | (SEQ ID NO: 2) | |
| sgCiDE-3 | GCCTGTAATCCCAGCACTTT | 216,087 |
| | (SEQ ID NO: 3) | |

### Example 11: Targeting Repetitive Genomic DNA Improves Glioblastoma Cell Elimination

To evaluate cell depletion by genomic shredding, U-251 glioblastoma cells that expressed Cas9 were transduced with a vector coding for mCherry and a single guide RNA targeting a selected repetitive genomic sequence or selected essential genes. After an eight-twelve hours incubation, mCherry expression was measured.

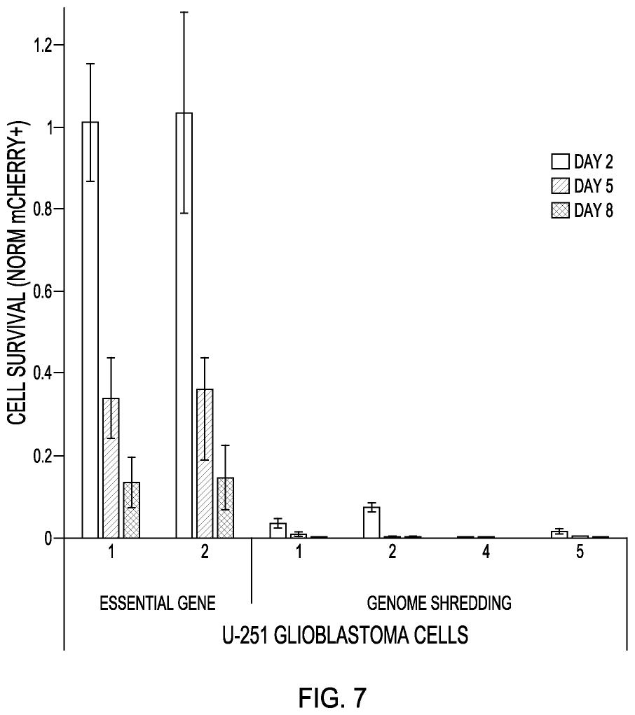

FIG. 7 illustrates that less glioblastoma cell survival was observed when the guide RNAs were targeted to repetitive genomic DNA than to essential genes.

### Example 12: Targeting Repetitive Genomic DNA Improves Elimination of Different Cancer Cell Types

HEK293, HAP1, A549, and U-251 cells were stably transduced with a lentiviral vector (pCF226) to express Cas9 (HEK-pCF226, HAP1-pCF226, A549-pCF226, and U251-pCF226). These cells were also stably transduced to express mCherry fluorescence marker.

HEK-pCF226 cells are cells from the human embryonic kidney HEK293T cell line that express Cas9. HAP1-pCF226 cells are cells derived from the human KBM7 cell line (Carette et al., Ebola virus entry requires the cholesterol transporter Niemann-Pick C1. Nature (2011)) that express Cas9. A549-pCF226 cells are cells from the human lung cancer A549 cell line that express Cas9. U251-pCF226 cells are cells from the human glioblastoma cell line U-251 that express Cas9.

The effect of guide RNA expression on cell viability was assessed using a competitive proliferation assay in which cells expressing a specific sgRNA (Table 2), coupled to mCherry expression from the same vector, were mixed with parental cells expressing only Cas9 WT, and the mCherry-positive population was followed over time. The sgRNAs used targeted a neutral gene (sgOR2B6), an essential gene (sgRPA1), greater, and a non-targeting control (sgNT) were compared

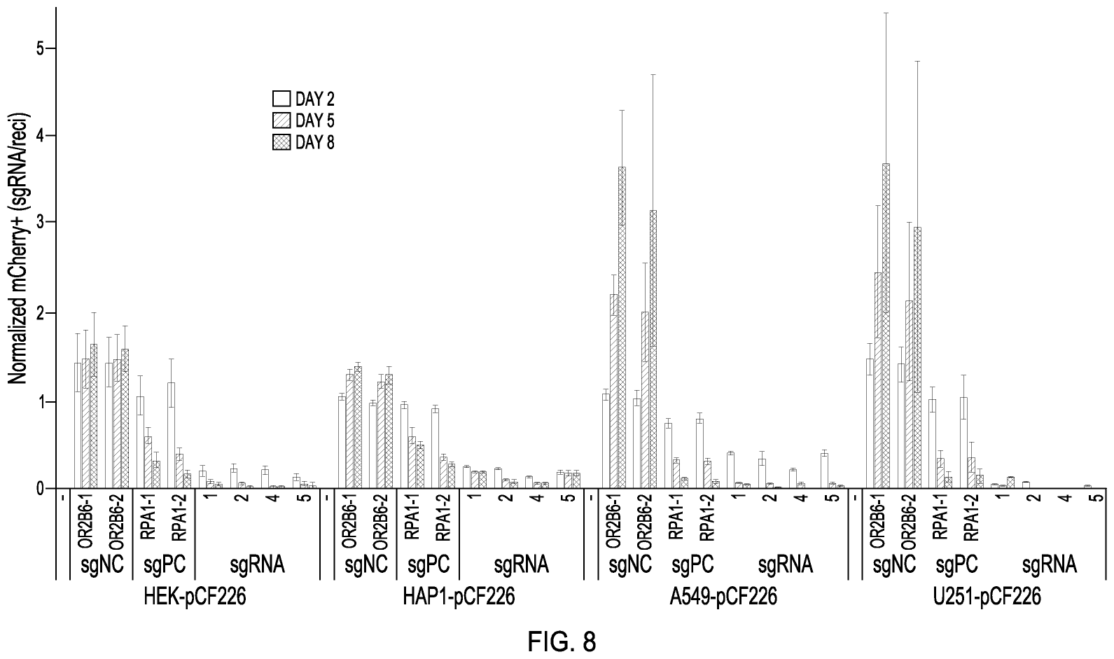

FIG. 8 illustrates that the CRISPR-Cas genome shredding methods and sgRNAs described herein rapidly and efficiently eliminated the targeted embryonic kidney cells and cancer cells in culture. Target cell elimination was more rapid when repetitive sequences were targeted than when essential genes such as the replication protein A1 (RPA1) were targeted.

### Example 13: Glioblastoma Cell Death Induced by Targeting Repetitive Genomic Sites

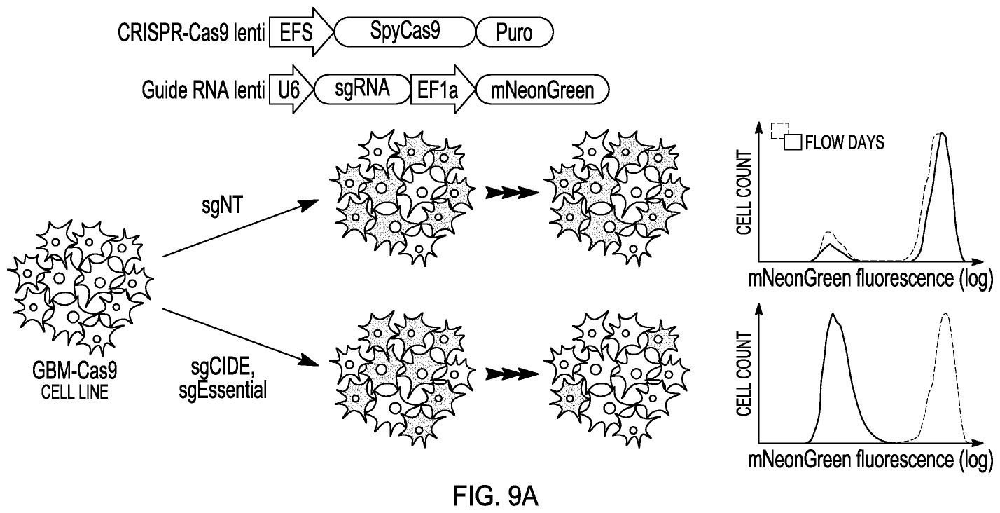

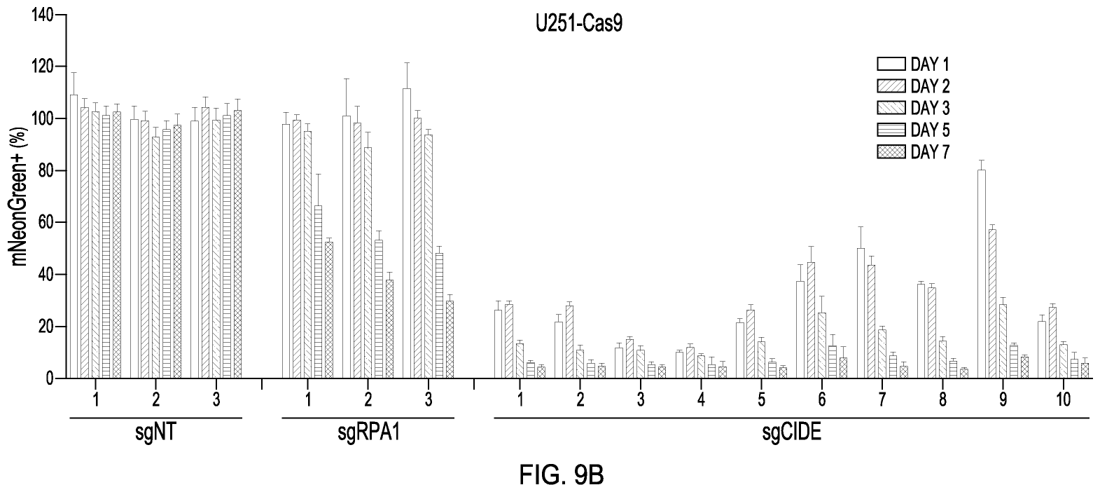

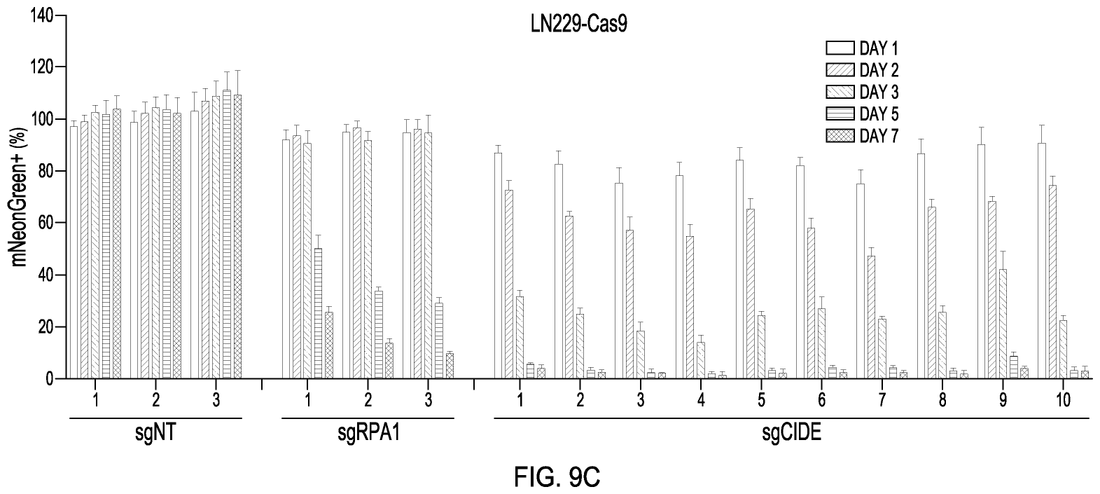

To assess timing and dynamic effects of genome shredding on glioblastoma cells in more detail, fluorescence time-lapse video microscopy was used to monitor Cas9-expressing U-251 cells stably transduced with lentivirus that expressed GFP-coupled sgCIDEs (sgCIDE-1/2/3/6/8/10) or negative controls (sgNT-1/2/3) over seven days. A schematic diagram of this system is shown in FIG. 9A.

Cell confluency quantification and propidium iodide (PI) staining revealed that sgCIDEs induced growth inhibition starting at day one (1) post-transduction, and cell death started as early as day two. To look at the genomic effects of repetitive loci targeting, DNA from lysed targeted cells was separated on agarose-coated slides. Single-cell analysis of Cas9 expressing U-251 and LN-229 using comet assays showed that the DNA from sgCIDE-1/2/3 expressing cells exhibited very long tails at 24 hours post-transduction compared to control (sgNT-1/2/3). These results indicated that extensive genomic fragmentation had occurred even at this early timepoint (24 hours).

Competitive proliferation assays were performed with Cas9-expressing U251 and LN229 glioblastoma cell lines. Wild type cells not expressing Cas9 were used for normalization. The cell lines were stably transduced with the guide RNAs inducing genome shredding (sgCIDE1-10, Table 2), guide RNAs targeting an essential gene (sgRPA1), or a control non-targeting guide RNA (sgNT). The changes in ratios of sgRNA-transduced cells (mNeonGreen+) were monitored by flow cytometry over seven days.

Cell lines (U-251, LN-18) were stably transduced with a lentiviral vector expressing Cas9 (pCF226) and selected on puromycin (1.0-2.0 μg/ml). Subsequently, Cas9 expressing cell lines were further stably transduced with pairs of lentiviral vectors (pCF221) expressing various mNeonGreen-tagged sgRNAs. Volume of virus was adjusted as appropriate between cell lines to establish similar levels of infectivity, with ˜2× more virus used in LN-18 cells than U-251 cells. At day two post-transduction, sgRNA expressing populations were mixed approximately 80:20 with parental cells and the fraction of mNeonGreen-positive cells was quantified over time by flow cytometry (Attune NxT flow cytometer, Thermo Fisher Scientific). The changes in ratios of sgRNA-transduced cells (mNeonGreen+) were monitored by flow cytometry over seven days.

As illustrated in FIG. 9B-9C, expression of the genome shredding guide RNAs (sgCIDE1-10) quickly destroyed the U251 and LN229 glioblastoma cells, while expression of the essential gene guide RNA led to substantially less cell death, compared to the non-targeting (control) guide RNAs.

Hence CRISPR-Cas genome shredding through targeting of highly repetitive sequences in the genome is a robust strategy for rapid and efficient elimination of cancer cells such as glioblastoma cells. Notably, targeting of repetitive sequences largely surpassed the efficacy of CRISPR-Cas9 methods directed at targeting of a key essential gene, highlighting the power of this approach.

### Example 14: Repetitive Loci are Spread Throughout Organisms' Genomes

Given the efficiency of genome shredding-based cell elimination, the origin and distribution of repetitive and highly repetitive CRISPR-Cas9 target loci in the genome was examines. To distinguish genome-specific versus general sequences, the inventors compared repetitive element from the human (Homo sapiens, hg38), mouse (Mus musculus, mm10), and chicken (Gallus gallus, galGal6) genomes, and annotated each sequence with over a thousand repeats in either of the three genomes. Genomic mapping of repeat elements demonstrated nearly uniform distribution throughout each genome, with the exception of a few regions that were devoid of repetitive guide RNA targets. When compared to annotated databases, the most common repeat sequences in the human genome mapped to retrotransposons and other mobile genetic elements (MGEs). While these MGE-targeting guide RNAs are species-specific, as is common for retrotransposons, a second class of highly repetitive target loci was represented by repeat expansion motifs. Repeat expansions can accumulate and expand in genomes because of replication errors in regions with specific repeat k-mer motifs. Not surprising due to the simplicity of these motifs, matching repeat expansion targets were identified across all three genomes. Parallel competitive proliferation assays in Cas9 expressing human U-251 glioblastoma, mouse GL261 glioblastoma, and chicken DF-1 fibroblast cells confirmed that repeat expansion targeting pan-vertebrate sgCIDEs rapidly induce depletion of transduced cells independent of their genetic origin.

### Example 15: Genome Shredding is Genotype Agnostic

The alkylating agent temozolomide (TMZ) is the current frontline chemotherapy for GBM but is only effective in cells when promoter methylation of O-6-methylguanine-DNA methyltransferase (MGMT) silences its expression. This is because active MGMT removes the TMZ-added methyl group from the O⁶ position of guanine, rendering the treatment ineffective. In sensitive glioblastoma cells, TMZ leads to a prolonged G2/M arrest followed by a p53-dependent cell death. This Example illustrates CRISPR-Cas9 genome shredding compared to chemotherapy in TMZ-sensitive and TMZ-resistant glioblastoma cells.

To investigate the speed of cell elimination by either method, Cas9 expressing TMZ-sensitive U-251 and LN-229, and TMZ-resistant T98G and LN-18, glioblastoma cells were treated with TMZ or these cells were transduced with lentiviral vectors expressing sgCIDEs.

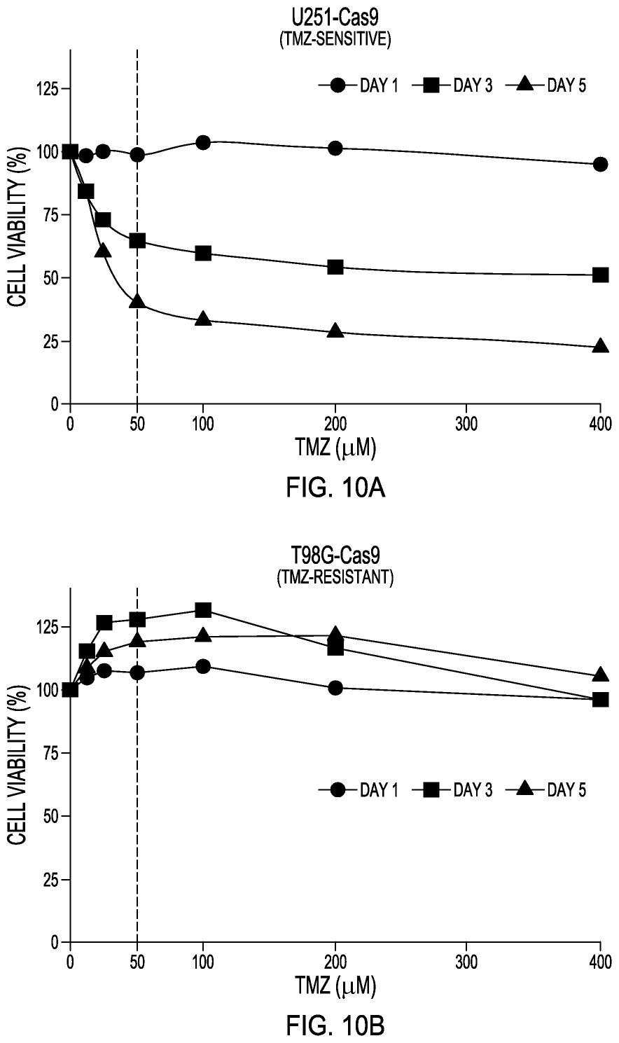

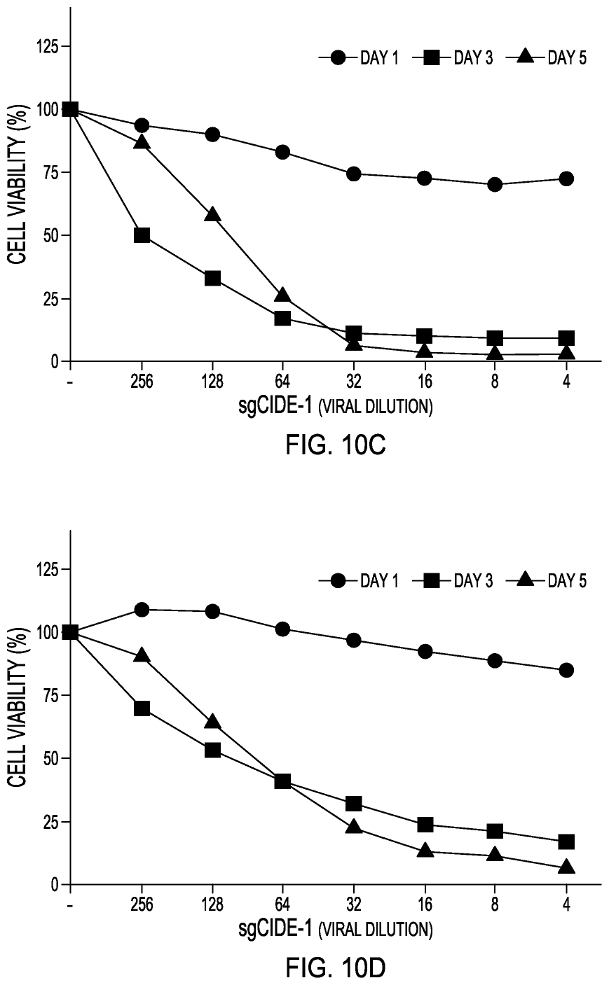

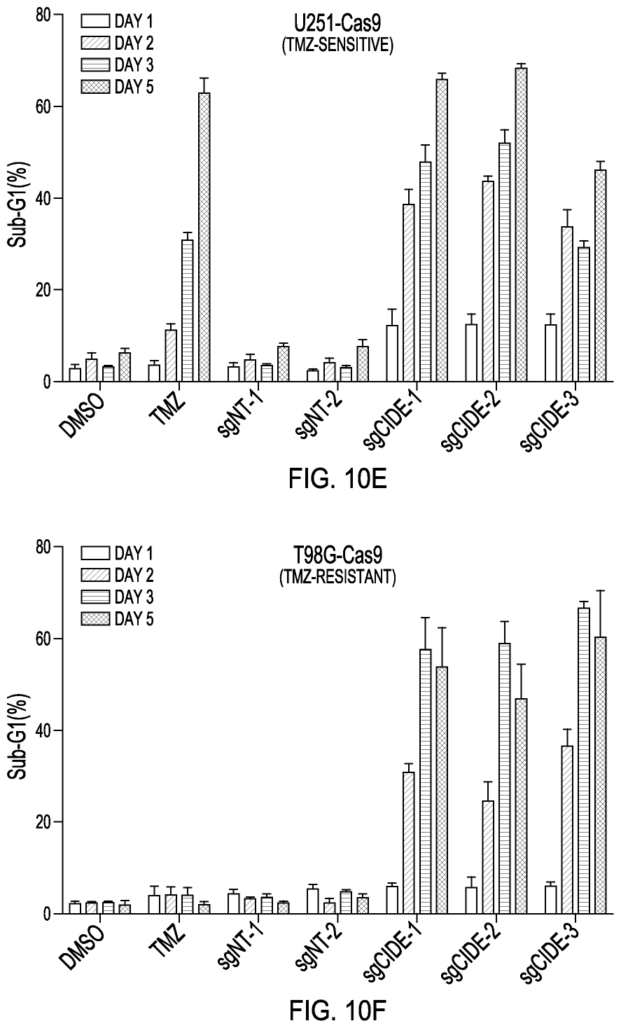

Luminescence-based quantification of cell viability over five days showed that lethality observed only in U-251 and LN-229 that were sensitive to TMZ (FIG. 10A-10B, 10E-10F). In contrast, sgCIDE-1/2/3/6/8/10 (Table 2) expression revealed viral titer-dependent lethality in all four tested glioblastoma cell lines independent of MGMT promoter methylation status and sensitivity to chemotherapy, while negative controls (sgNT-1/2/3) showed no effect (FIG. 10C-10F). Additionally, timing of viability loss was much quicker for genome shredding, with strong lethality already on day three, compared to TMZ that induced only weak-to-medium effects at day three even for TMZ-sensitive LN-229 and U-251 GBM cells.

The effects of genome shredding on cell cycle progression were then assessed. Cells were treated with TMZ or sgCIDEs for one to five days and then stained with PI after fixation for analysis by flow cytometry. Control DMSO and sgNT-1/2 treatments, as well as guide RNAs targeting an olfactory receptor (sgOR2B6-1/2), showed comparable normal cell cycle profiles in Cas9 expressing U-251, LN-229, T98G, and LN-18 glioblastoma cells. TMZ-sensitive glioblastoma cells treated with TMZ (50 μM or 100 μM) exhibited G2/M arrest with initial increase of the G2 peak, loss of G1, and slow increase of the Sub-G1 (apoptotic) population starting at day two. Increases of the Sub-G1 population was more prominent in TP53-mutant U-251 cells compared LN-229 with wild-type TP53, consistent with previous observations that TP53 status affects resolution of the G2/M arrest. Treatment with guide RNAs targeting the essential gene RPA1 (sgRPA1-2/3) resulted in an accumulation in S-phase starting at day three, accompanied by increase of the Sub-G1 population, in all four glioblastoma cell lines. See FIGS. 10E-10F.

In contrast, genome shredding with sgCIDE-1/2/3/6/8/10 led to a rapid increase of the Sub-G1 population starting at day one post-transduction, combined with a drastic depletion of the G1 peak and slight increase of the S-phase population, in all four tested glioblastoma cell lines. Noteworthy, this change in cell cycle profile was consistent across all six sgCIDEs, for all four tested GBM cell lines independent of MGMT promoter methylation and TERT promoter or TP53 mutational status, indicating a characteristic path to cell death. At day two post-transduction, the Sub-G1 population of sgCIDE transduced samples already represented approximately 20-40% of cells, and by day 3 the Sub-G1 population was 30-60%. See FIGS. 10E-10F. Hence, genome shredding leads to more cell death than TMZ treated samples even in chemotherapy-sensitive cell lines.

Together, CRISPR-Cas genome shredding was both more rapid than TMZ at inducing cell death and it was effective independent of the GBM cells' genetic and epigenetic makeup. Hence, genome shredding can be more versatile when addressing intratumoral cellular heterogeneity issues.

### Example 16: Genome Shredding is Difficult to Escape

Because recurrent tumors develop from cells that escape treatment, either by avoiding exposure, tolerating the effects, or developing resistance, colony formation assays were performed to evaluate the robustness of CRISPR-Cas genome shredding in eliminating target cells.

TMZ-resistant LN-229 cell lines were isolated to determine which types of treatments could overcome such resistance. Cas9 expressing U-251, LN-229, T98G, and LN-18 cells were stably transduced with lentiviral vectors expressing sgNT-1/2 or sgCIDE-1/2/3/6/8/10 (Table 2), and seeded at 100, 1,000, and 10,000 cells per 6-well plate. Control cells were treated with DMSO or TMZ (50 μM).

Crystal violet staining two weeks later revealed that TMZ treatment reduced colony numbers by about two log-scales compared to DMSO in U-251 and LN-229 cells only, while T98G and LN-18 cells were unaffected as expected. Treatment with sgNT-1/2 had little effect on colony formation. Conversely, genome shredding by sgCIDE-1/2/3/6/8/10 expression led to an over three log-scales reduction in colony count across all four tested GBM cell lines. Hence, under the tested conditions, CRISPR-Cas genome shredding was more than 10-fold efficient at eliminating GBM cells compared to TMZ in chemotherapy-sensitive cell lines.

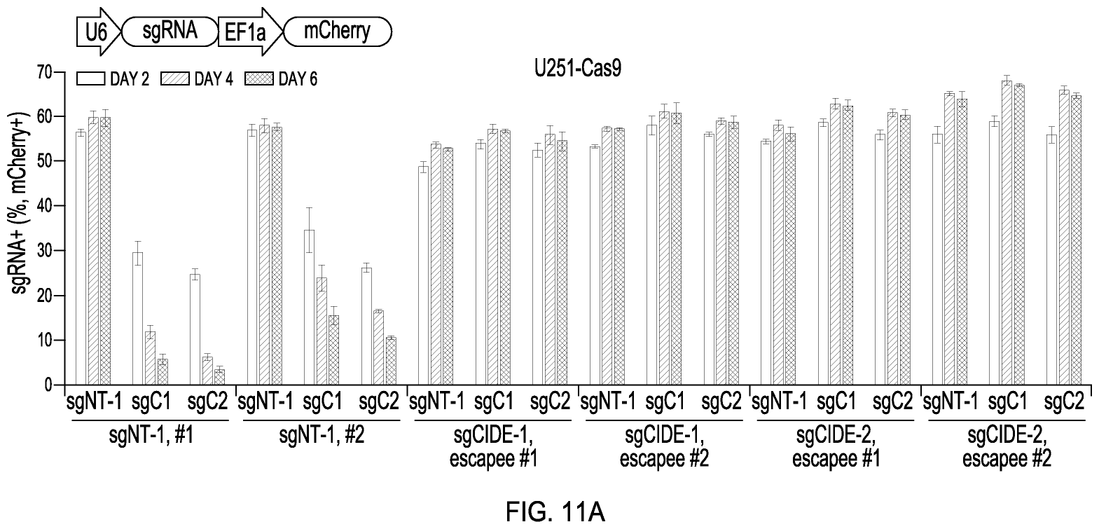

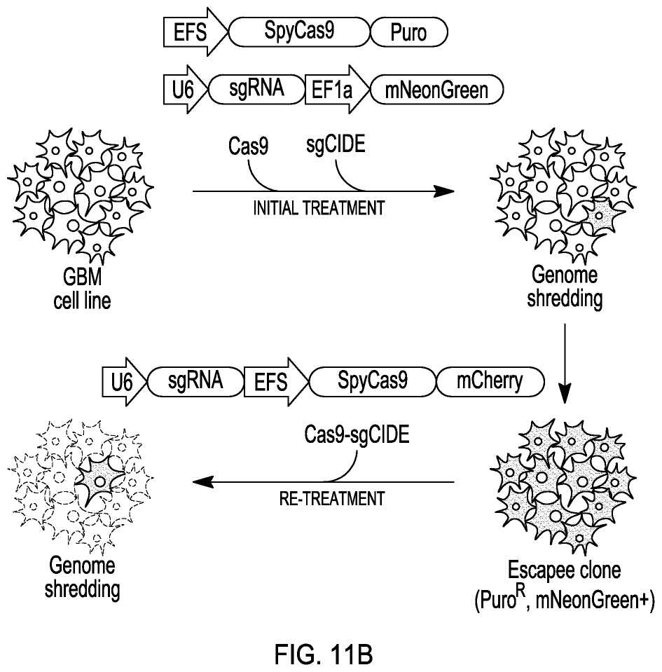

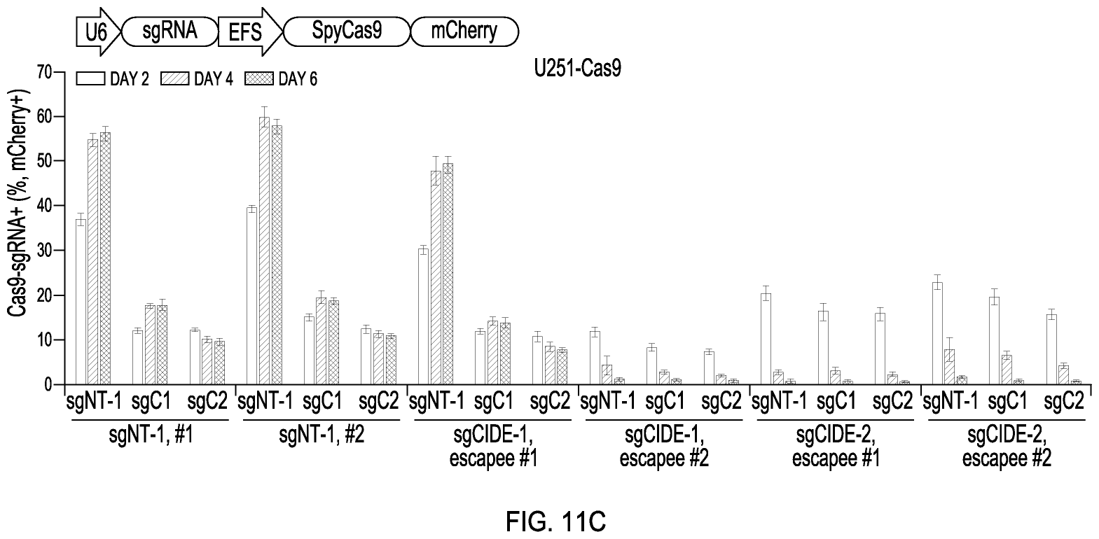

A small percentage of Cas-9 glioblastoma cells appeared to escape genome shredding when transduced with the sgRNA expression cassette shown in FIG. 11A. For example, sgC1, sgCIDE-1, sgC2, sgCIDE-2 escapee cell lines were cloned from U251-Cas9 cells that escaped a first round of CRISPR-Cas genome shredding. When re-tested by re-introducing just the sgCIDE expression vector (U6-sgRBA-EF1a-mCherry), these escapee cell lines again exhibited resistance to genomic shredding (FIG. 11A). However, up to 95% or more cell depletion of such U251-Cas9 escapee clones was observed after treatment with an all-in-one vector (pCF826, FIG. 11C) expressing both the Cas9 and the sgCIDE. Hence, as shown in FIG. 11B, introducing the Cas9 nuclease separately from the sgCIDE may allow escape of genome shredding in a small number of cells, but introducing both the Cas9 nuclease with the sgCIDE leads to even greater percentage cell depletion (FIG. 11C). An example of a single expression vector that expresses both Cas9 and an sgRNA (sgCIDE) is shown in FIG. 11C.

### Example 17: Reducing Glioblastoma Burden In Vivo

The proof-of-concept studies described above were all carried out with pre-engineered cell lines stably expressing Cas9 and guide RNAs from lentiviral vectors. To assess the therapeutic potential of CRISPR-Cas genome shredding, orthotopic intracranial glioblastoma xenograft models were established that provided local delivery of CRISPR-Cas9 after establishment of tumors. Direct delivery of Cas9-sgRNA ribonucleoprotein (RNP) complexes, rather than viral vectors encoding those components, can reduce toxicities of persistent viral transductions and integrational mutagenesis, but may suffer low efficacy.

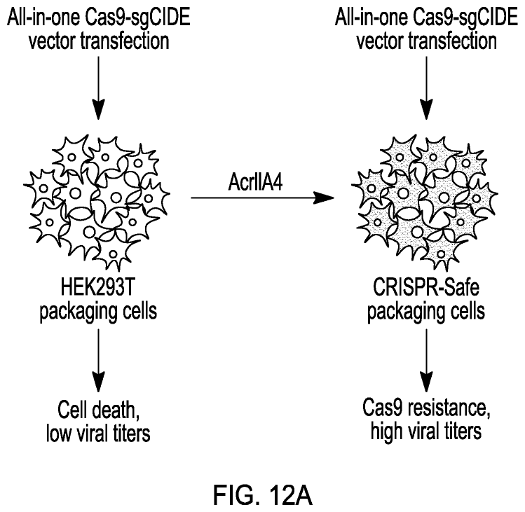

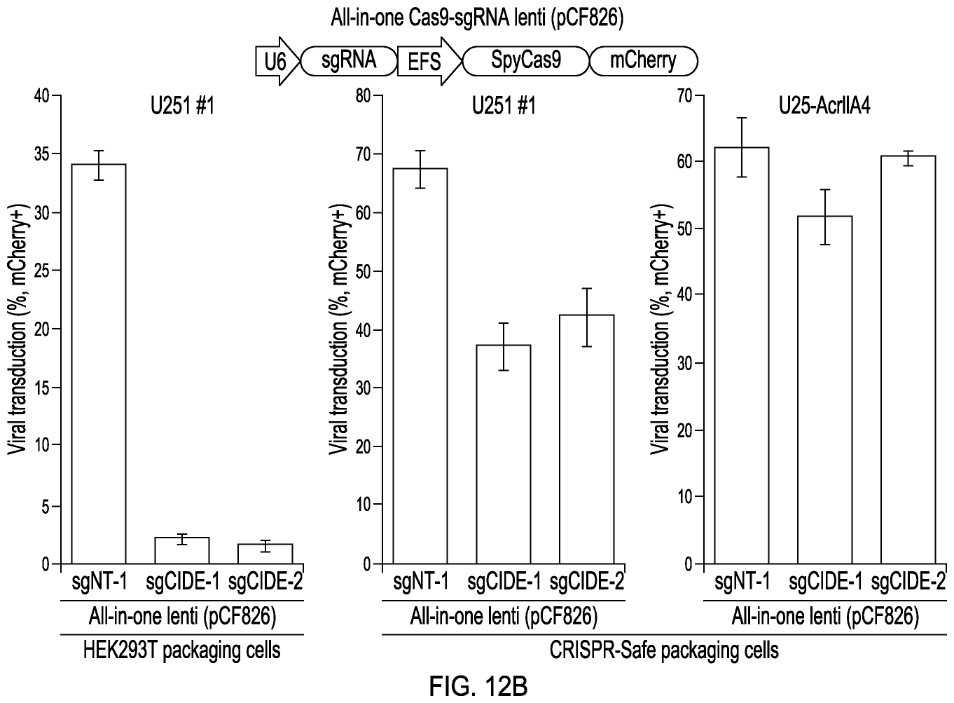

To leverage high viral delivery efficiencies, virus-like particles (VLPs) can be used as Cas9 RNP carriers. Hence, a murine leukemia virus (MLV)-based system of VLPs was adopted for local Cas9 RNP delivery (Mangeot et al., Nat. Commun. 10, 45 (2019)). Vector-based improvements in guide RNA and Cas9 expression so that both are expressed in target cells (FIG. 12A) led to an overall 60-80-fold increase in editing efficiency compared to the original system. Even with 5-fold diluted Cas9-sgCIDE expression vector, the optimized Cas9-RNP delivery method enabled over 95% editing efficiency of a polyclonal mCherry expressing LN-229 glioblastoma cell line.

Genome shredding efficiency was then assessed in wild-type U-251 and LN-229 glioblastoma cells upon VLP-based delivery of Cas9 and negative control sgNT-1/3 or sgCIDE-1/3. Parental U251 cells (U251-pCF226-pCF821-sgNT-1 #1) and U251 cells that stably expressed AcrIIA4 (pCF525-AcrIIA4) were transduced with all-in-one lentiviral vectors (pCF826) expressing an mCherry-tagged Cas9 and sgCIDE1, sgCIDE2 or control non-targeting sgNT-1 sgRNAs. Viral particles were produced using either standard HEK293T packaging cells or the CRISPR-Safe packaging cell line. Viral titers were assessed by flow cytometry-based quantification of mCherry expression at day two post-transduction.

As illustrated in FIG. 12B, analysis of viral transduction (% mCherry-expressing cells) at day 2 post-treatment demonstrated that use of the CRISPR-Safe viral packaging cell line rescued viral titers of all-in-one Cas9-sgCIDE vectors. Hence, a single expression vector can be used to produce both the Cas9 nuclease and the sgRNAs of interest.

### REFERENCES

Ade, J., DeYoung, B. J., Golstein, C., and Innes, R. W. (2007). Indirect activation of a plant nucleotide binding site-leucine-rich repeat protein by a bacterial protease. Proc. Natl. Acad. Sci. USA 104, 2531-2536.
Alfano, J. R., and Collmer, A. (2004). Type III secretion system effector proteins: double agents in bacterial disease and plant defense. Annu. Rev. Phytopathol. 42, 385-414.
Anders, S., and Huber, W. (2010). Differential expression analysis for sequence count data. Genome Biol. 11, R106.
Anders, C., Niewoehner, O., Duerst, A., and Jinek, M. (2014). Structural basis of PAM-dependent target DNA recognition by the Cas9 endonuclease. Nature 513, 569-573.
Baltes, N. J., Hummel, A. W., Konecna, E., Cegan, R., Bruns, A. N., Bisaro, D. M., and Voytas, D. F. (2015). Conferring resistance to geminiviruses with the CRISPR-Cas prokaryotic immune system. Nat. Plants 1, 15145.
Beernink, P. T., Yang, Y. R., Graf, R., King, D. S., Shah, S. S., and Schachman, H. K. (2001). Random circular permutation leading to chain disruption within and near alpha helices in the catalytic chains of aspartate transcarbamoylase: effects on assembly, stability, and function. Protein Sci. 10, 528-537.
Bera, A. K., Kuhn, R. J., and Smith, J. L. (2007). Functional characterization of cis and trans activity of the Flavivirus NS2B-NS3 protease. J. Biol. Chem. 282, 12883-12892.
Brinkman, E. K., Chen, T., Amendola, M., and van Steensel, B. (2014). Easy quantitative assessment of genome editing by sequence trace decomposition. Nucleic Acids Res. 42, e168.
Butler, J. S., Mitrea, D. M., Mitrousis, G., Cingolani, G., and Loh, S. N. (2009). Structural and thermodynamic analysis of a conformationally strained circular permutant of barnase. Biochemistry 48, 3497-3507.
Carette, J. E., Raaben, M., Wong, A. C., Herbert, A. S., Obernosterer, G., Mulherkar, N., Kuehne, A. I., Kranzusch, P. J., Griffin, A. M., Ruthel, G., et al. (2011). Ebola virus entry requires the cholesterol transporter Niemann-Pick C1. Nature 477, 340-343.
Chaparro-Garcia, A., Kamoun, S., and Nekrasov, V. (2015). Boosting plant immunity with CRISPR/Cas. Genome Biol. 16, 254.
Chavez, A., Scheiman, J., Vora, S., Pruitt, B. W., Tuttle, M., P R Iyer, E., Lin, S., Kiani, S., Guzman, C. D., Wiegand, D. J., et al. (2015). Highly efficient Cas9-mediated transcriptional programming. Nat. Methods 12, 326-328.
Chen, B., Gilbert, L. A., Cimini, B. A., Schnitzbauer, J., Zhang, W., Li, G.-W., Park, J., Blackburn, E. H., Weissman, J. S., Qi, L. S., and Huang, B. (2013). Dynamic imaging of genomic loci in living human cells by an optimized CRISPR/Cas system. Cell 155, 1479-1491.
Chisholm, S. T., Dahlbeck, D., Krishnamurthy, N., Day, B., Sjolander, K., and Staskawicz, B. J. (2005). Molecular characterization of proteolytic cleavage sites of the Pseudomonas syringae effector AvrRpt2. Proc. Natl. Acad. Sci. USA 102, 2087-2092.
Cong, L., Ran, F. A., Cox, D., Lin, S., Barretto, R., Habib, N., Hsu, P. D., Wu, X., Jiang, W., Marraffini, L. A., and Zhang, F. (2013). Multiplex genome engineering using CRISPR/Cas systems. Science 339, 819-823.
Coradetti, S. T., Pinel, D., Geiselman, G. M., Ito, M., Mondo, S. J., Reilly, M. C., Cheng, Y.-F., Bauer, S., Grigoriev, I. V., Gladden, J. M., et al. (2018). Functional genomics of lipid metabolism in the oleaginous yeast Rhodosporidium toruloides. eLife 7, e32110.
Davis, K. M., Pattanayak, V., Thompson, D. B., Zuris, J. A., and Liu, D. R. (2015). Small molecule-triggered Cas9 protein with improved genome-editing specificity. Nat. Chem. Biol. 11, 316-318.
Fellmann, C., Hoffmann, T., Sridhar, V., Hopfgartner, B., Muhar, M., Roth, M., Lai, D. Y., Barbosa, I. A. M., Kwon, J. S., Guan, Y., et al. (2013). An optimized microRNA backbone for effective single-copy RNAi. Cell Rep. 5, 1704-1713.
Fellmann, C., Gowen, B. G., Lin, P.-C., Doudna, J. A., and Corn, J. E. (2017). Cornerstones of CRISPR-Cas in drug discovery and therapy. Nat. Rev. Drug Discov. 16, 89-100.
Gao, M., Matusick-Kumar, L., Hurlburt, W., DiTusa, S. F., Newcomb, W. W., Brown, J. C., McCann, P. J., 3rd, Deckman, I., and Colonno, R. J. (1994). The protease of herpes simplex virus type 1 is essential for functional capsid formation and viral growth. J. Virol. 68, 3702-3712.
Gaudelli, N. M., Komor, A. C., Rees, H. A., Packer, M. S., Badran, A. H., Bryson, D. I., and Liu, D. R. (2017). Programmable base editing of A, T to G, C in genomic DNA without DNA cleavage. Nature 551, 464-471.
Gilbert, L. A., Horlbeck, M. A., Adamson, B., Villalta, J. E., Chen, Y., Whitehead, E. H., Guimaraes, C., Panning, B., Ploegh, H. L., Bassik, M. C., et al. (2014).
Genome-scale CRISPR-mediated control of gene repression and activation. Cell 159, 647-661.
Guilinger, J. P., Thompson, D. B., and Liu, D. R. (2014). Fusion of catalytically inactive Cas9 to FokI nuclease improves the specificity of genome modification. Nat. Biotechnol. 32, 577-582.
Hartmann, S., and Lucius, R. (2003). Modulation of host immune responses by nematode cystatins. Int. J. Parasitol. 33, 1291-1302.
Hemphill, J., Borchardt, E. K., Brown, K., Asokan, A., and Deiters, A. (2015). Optical control of CRISPR/Cas9 gene editing. J. Am. Chem. Soc. 137, 5642-5645.
Hilton, I. B., D'Ippolito, A. M., Vockley, C. M., Thakore, P. I., Crawford, G. E., Reddy, T. E., and Gersbach, C. A. (2015). Epigenome editing by a CRISPRCas9-based acetyltransferase activates genes from promoters and enhancers. Nat. Biotechnol. 33, 510-517.
Jinek, M., Chylinski, K., Fonfara, I., Hauer, M., Doudna, J. A., and Charpentier, E. (2012). A programmable dual-RNA-guided DNA endonuclease in adaptive bacterial immunity. Science 337, 816-821.
Jinek, M., East, A., Cheng, A., Lin, S., Ma, E., and Doudna, J. (2013). RNA-programmed genome editing in human cells. eLife 2, e00471.
Johnson, R. J., Lin, S. R., and Raines, R. T. (2006). A ribonuclease zymogen activated by the NS3 protease of the hepatitis C virus. FEBS J. 273, 5457-5465.
Jones, A. M., Mehta, M. M., Thomas, E. E., Atkinson, J. T., Segall-Shapiro, T. H., Liu, S., and Silberg, J. J. (2016). The structure of a thermophilic kinase shapes fitness upon random circular permutation. ACS Synth. Biol. 5, 415-425.
Kennedy, E. M., Kornepati, A. V. R., Goldstein, M., Bogerd, H. P., Poling, B. C., Whisnant, A. W., Kastan, M. B., and Cullen, B. R. (2014). Inactivation of the human papillomavirus E6 or E7 gene in cervical carcinoma cells by using a bacterial CRISPR/Cas RNA-guided endonuclease. J. Virol. 88, 11965-11972.
Kim, S. H., Qi, D., Ashfield, T., Helm, M., and Innes, R. W. (2016). Using decoys to expand the recognition specificity of a plant disease resistance protein. Science 351, 684-687.
Kim, K., Park, S. W., Kim, J. H., Lee, S. H., Kim, D., Koo, T., Kim, K.-E., Kim, J. H., and Kim, J.-S. (2017). Genome surgery using Cas9 ribonucleoproteins for the treatment of age-related macular degeneration. Genome Res. 27, 419-426.
Komor, A. C., Kim, Y. B., Packer, M. S., Zuris, J. A., and Liu, D. R. (2016). Programmable editing of a target base in genomic DNA without double-stranded DNA cleavage. Nature 533, 420-424.
Kummerer, B. M., Amberg, S. M., and Rice, C. M. (2013). Flavivirin. In Handbook of Proteolytic Enzymes, N. D. Rawlings and G. Salvesen, eds. (Academic Press), pp. 3112-3120.
Mali, P., Yang, L., Esvelt, K. M., Aach, J., Guell, M., DiCarlo, J. E., Norville, J. E., and Church, G. M. (2013). RNA-guided human genome engineering via Cas9. Science 339, 823-826.
Mehta, M. M., Liu, S., and Silberg, J. J. (2012). A transposase strategy for creating libraries of circularly permuted proteins. Nucleic Acids Res. 40, e71.
Mehta, D., Sturchler, A., Hirsch-Hoffmann, M., Gruissem, W., and Vanderschuren, H. (2018). CRISPR-Cas9 interference in cassava linked to the evolution of editing-resistant geminiviruses. bioRxiv. See: doi.org/10.1101/314542.
Oakes, B. L., Nadler, D. C., and Savage, D. F. (2014). Protein engineering of Cas9 for enhanced function. Methods Enzymol. 546, 491-511.
Oakes, B. L., Nadler, D. C., Flamholz, A., Fellmann, C., Staahl, B. T., Doudna, J. A., and Savage, D. F. (2016). Profiling of engineering hotspots identifies an allosteric CRISPR-Cas9 switch. Nat. Biotechnol. 34, 646-651.
Park, H. M., Liu, H., Wu, J., Chong, A., Mackley, V., Fellmann, C., Rao, A., Jiang, F., Chu, H., Murthy, N., and Lee, K. (2018). Extension of the crRNA enhances Cpf1 gene editing in vitro and in vivo. Nat. Commun. 9, 3313.
Perez, A. R, Pritykin, Y., Vidigal, J. A., Chhangawala, S., Zamparo, L., Leslie, C. S., and Ventura, A. (2017). GuideScan software for improved single and paired CRISPR guide RNA design. Nat. Biotechnol. 35, 347-349.
Plainkum, P., Fuchs, S. M., Wiyakrutta, S., and Raines, R. T. (2003). Creation of a zymogen. Nat. Struct. Biol. 10, 115-119.
Qi, L. S., Larson, M. H., Gilbert, L. A., Doudna, J. A., Weissman, J. S., Arkin, A. P., and Lim, W. A. (2013). Repurposing CRISPR as an RNA-guided platform for sequence-specific control of gene expression. Cell 152, 1173-1183.
Qian, Z., and Lutz, S. (2005). Improving the catalytic activity of Candida antarctica lipase B by circular permutation. J. Am. Chem. Soc. 127, 13466-13467.
Ramanathan, M. P., Chambers, J. A., Pankhong, P., Chattergoon, M., Attatippaholkun, W., Dang, K., Shah, N., and Weiner, D. B. (2006). Host cell killing by the West Nile Virus NS2B-NS3 proteolytic complex: NS3 alone is sufficient to recruit caspase-8-based apoptotic pathway. Virology 345, 56-72.
Richter, F., Fonfara, I., Gelfert, R, Nack, J., Charpentier, E., and Moglich, A. (2017). Switchable Cas9. Curr. Opin. Biotechnol. 48, 119-126.
Roybal, K. T., Rupp, L. J., Morsut, L., Walker, W. J., McNally, K. A., Park, J. S., and Lim, W. A. (2016). Precision tumor recognition by T cells with combinatorial antigen-sensing circuits. Cell 164, 770-779.
Sanjana, N. E., Shalem, O., and Zhang, F. (2014). Improved vectors and genome-wide libraries for CRISPR screening. Nat. Methods 11, 783-784.
Seon Han, J., Kim, D.-H., and Yong Choi, K. (2013). Potyvirus NIa protease. In Handbook of Proteolytic Enzymes, N. D. Rawlings and G. Salvesen, eds. (Academic Press), pp. 2427-2432.
Skern, T. (2013). Picornain 3C. In Handbook of Proteolytic Enzymes, N. D. Rawlings and G. Salvesen, eds. (Academic Press), pp. 2396-2402.
Staahl, B. T., Benekareddy, M., Coulon-Bainier, C., Banfal, A. A., Floor, S. N., Sabo, J. K., Urnes, C., Munares, G. A., Ghosh, A., and Doudna, J. A. (2017). Efficient genome editing in the mouse brain by local delivery of engineered Cas9 ribonucleoprotein complexes. Nat. Biotechnol. 35, 431-434.
Tanenbaum, M. E., Gilbert, L. A., Qi, L. S., Weissman, J. S., and Vale, R. D. (2014). A protein-tagging system for signal amplification in gene expression and fluorescence imaging. Cell 159, 635-646.
Tomlinson, K. R., Bailey, A. M., Alicai, T., Seal, S., and Foster, G. D. (2018). Cassava brown streak disease: historical timeline, current knowledge and future prospects. Mol. Plant Pathol. 19, 1282-1294.
Tsai, S. Q., Wyvekens, N., Khayter, C., Foden, J. A., Thapar, V., Reyon, D., Goodwin, M. J., Aryee, M. J., and Joung, J. K. (2014). Dimeric CRISPR RNA-guided FokI nucleases for highly specific genome editing. Nat. Biotechnol. 32, 569-576.
Wang, T., Birsoy, K., Hughes, N. W., Krupczak, K. M., Post, Y., Wei, J. J., Lander, E. S., and Sabatini, D. M. (2015). Identification and characterization of essential genes in the human genome. Science 350, 1096-1101.
Whitehead, T. A., Bergeron, L. M., and Clark, D. S. (2009). Tying up the loose ends: circular permutation decreases the proteolytic susceptibility of recombinant proteins. Protein Eng. Des. Sel. 22, 607-613.
Yu, Y., and Lutz, S. (2011). Circular permutation: a different way to engineer enzyme structure and function. Trends Biotechnol. 29, 18-25.
Zuris, J. A., Thompson, D. B., Shu, Y., Guilinger, J. P., Bessen, J. L., Hu, J. H., Maeder, M. L., Joung, J. K., Chen, Z.-Y., and Liu, D. R. (2015). Cationic lipid-mediated delivery of proteins enables efficient protein-based genome editing in vitro and in vivo. Nat. Biotechnol. 33, 73-80.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### Statements:

1. A guide RNA that binds specifically to a repetitive DNA sequence in a cell.
2. The guide RNA of statement 1, which is a human cell, an animal cell, a plant cell, or a fungal cell.
3. The guide RNA of statement 1 or 2, with a sequence that includes a heterologous Protospacer Adjacent Motif (PAM).

What is claimed:

1. A composition comprising at least one Cas protein and at least one guide RNA that binds specifically to a repetitive DNA sequence in a cell.

2. The composition of claim 1, wherein the Cas protein is an active or deactivated nuclease, wherein the deactivated Cas nuclease is deactivated in the composition but activated in the cell.

3. The composition of claim 1, wherein the Cas protein is a circularly permuted Cas9 protein that is inactive until cleaved by a protease that specifically recognizes and cleaves a cleavage site in the circularly permuted Cas9 protein.

4. The composition of claim 3, wherein the Cas protein is a circularly permuted Cas protein, and where the circular permutation is in a helical domain, in a RuvC-III domain, or in a C-terminal domain (CTD).

5. The composition of claim 1, wherein the Cas protein has at least 90% sequence identity to any one of SEQ ID NO:38, 40-49 or 50.

6. The composition of claim 1, wherein the Cas protein's activity or expression is inducible.

7. The composition of claim 1, wherein the guide RNA's activity or expression is inducible.

8. The composition of claim 1, further comprising a carrier or targeting agent, where the carrier or targeting agent activates the Cas protein within, or delivers at least one Cas protein and at least one guide RNA to a specific cell type, or a combination thereof.

9. A kit comprising:
a. at least one guide RNA that binds specifically to a repetitive DNA sequence in a human cell;
b. at least one composition comprising a Cas protein and a guide RNA that binds specifically to a repetitive DNA sequence in a human cell;
c. at least one expression system comprising at least one expression cassette, each expression cassette comprising a promoter operably linked to a nucleic acid segment encoding a Cas nuclease, a guide RNA, or a combination thereof;
d. or a combination thereof, and
instructions for using the at least one RNA, the at least one composition, the at least one expression system, or a combination thereof for depleting an undesired cell type in a population of cells.

10. The kit of claim 9, wherein the cell is a human cell, an animal cell, a plant cell, or a fungal cell.

11. The kit of claim 9, wherein the population of cells is an in vitro cell culture.

12. The kit of claim 9, wherein the population of cells is in vivo within a subject.

13. The kit of claim 9, wherein the guide RNA comprises a sequence that has at least 90% sequence identity to any one of SEQ ID NO:1-37, 52-66.

14. The kit of claim 9, wherein the guide RNA further comprises a heterologous Protospacer Adjacent Motif (PAM).

15. The kit of claim 9, wherein the Cas protein is an active or deactivated nuclease.

16. The kit of claim 9, wherein the Cas protein is deactivated in the composition but activated in the cell.

17. The kit of claim 9, wherein the Cas protein is a circularly permuted Cas9 protein that is inactive until cleaved by a protease that specifically recognizes and cleaves a cleavage site in the circularly permuted Cas9 protein.

18. The kit of claim 9, wherein the Cas protein has at least 90% sequence identity to any one of SEQ ID NO:38, 40-49 or 50.

19. The kit of claim 9, wherein the Cas protein's activity or expression is inducible.

20. The kit of claim 9, wherein the guide RNA's activity or expression is inducible.

21. The kit of claim 9, wherein the promoter of the expression system is an inducible promoter.

22. The kit of claim 9, wherein the composition further comprises a carrier or targeting agent, where the targeting agent activates within a specific cell type, or delivers to a specific cell type, the at least one Cas nuclease, the at least one guide RNA, or a combination thereof.

23. The kit of claim 9, wherein the undesired cell type in a population of cells is a human, animal, plant, or a fungal cell type.

24. A method comprising contacting a cell with a composition comprising:
a. at least one guide RNA that binds specifically to a repetitive DNA sequence in a human cell;
b. at least one Cas protein and at least one guide RNA that binds specifically to a repetitive DNA sequence in a human cell;
c. at least one expression system comprising at least one expression cassette, each expression cassette comprising a promoter operably linked to a nucleic acid segment encoding a Cas protein, a guide RNA, or a combination thereof,
d. or a combination thereof.

25. The method of claim 24, wherein the circularly permuted Cas protein comprises an N-terminal segment of an original Cas protein fused in-frame at the original Cas protein's C-terminus.

26. The method of claim 25, wherein the circularly permuted Cas protein comprises a linker between the N-terminal segment and the original Cas protein's C-terminus.

27. The method of claim 25, wherein the circularly permuted Cas protein comprises a cleavable linker between the N-terminal segment and the original Cas protein's C-terminus.

28. The method of claim 27, wherein the linker comprises a sequence that is specifically recognized by a protease.

29. The method of claim 27, wherein the protease is expressed and/or is functional only in a targeted or selected cell type.

30. The method of claim 25, wherein the circularly permuted Cas protein is inactive until linker is cleaved.

31. The method of claim 25, wherein the at least one guide RNA has a sequence that has at least 90% sequence identity to any one of SEQ ID NO:1-37, 52-66.

32. The method of claim 25, wherein the guide RNA further comprises a heterologous Protospacer Adjacent Motif (PAM).

33. A method comprising administering the composition of claim 1 to a subject.

34. The method of claim 33, wherein the subject has or is suspected of having a cell proliferative disease or disorder.

35. The method of claim 34, wherein the cell proliferative disease or disorder is leukemia, polycythemia vera, lymphoma, Waldenstrom's macroglobulinemia, heavy chain disease, solid tumor, sarcoma, carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendothelio sarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, lung carcinoma, small cell lung carcinoma, bladder carcinoma, epithelial carcinoma, glioma, high-grade glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, retinoblastoma, or a combination thereof.

36. The method of claim 33, wherein the disease or disorder is a glioblastoma.

37. The composition of claim 1 formulated as a medicament.

38. The composition of claim 1 for use in the treatment of a cell proliferative disease or disorder.
